(19) United States
(12) Patent Application Publication
(10) Pub. No.: US 2023/0227848 A1
(43) Pub. Date: Jul. 20, 2023

(54) CORONAVIRUS VACCINE CONSTRUCTS AND METHODS OF MAKING AND USING SAME

(71) Applicant: Washington University, St. Louis, MO (US)

(72) Inventors: DAVID CURIEL, St. Louis, MO (US); Michael Diamond, St. Louis, MO (US); IGOR DMITRIEV, St. Louis, MO (US); Ahmed Hassan, St. Louis, MO (US)

(21) Appl. No.: 18/007,677

(22) PCT Filed: Jun. 1, 2021
(86) PCT No.: PCT/US21/35239

§ 371 Date: Dec. 1, 2022

Related U.S. Application Data
(60) Provisional application No. 63/032,815, filed on Jun. 1, 2020.
(60) Provisional application No. 63/051,103, filed on Jul. 13, 2020.
(60) Provisional application No. 63/162,417, filed on Mar. 17, 2021.

(51) Int. Cl. C12N 15/86; C07K 14/165; A61P 37/04

(52) U.S. Cl. CPC C12N 15/86; C07K 14/165; A61P 37/04; C12N 2710/10343; C12N 2770/20022; C12N 2770/20034

(57) ABSTRACT

Compositions and methods for treating a viral infection may comprise use of an adenoviral vector. An adenoviral vector of the present disclosure may comprise a non-human adenoviral genome with one or more gene locus functionally removed and a transgene. A method of treating a viral infection may comprise administering a composition comprising an adenoviral vector of the present disclosure, to a subject and reducing the infectivity or transmission of the virus. Intranasal administration provides enhance protection of the upper respiratory tract of a subject relative to intramuscular administration.

### CROSS REFERENCE TO RELATED APPLICATIONS

This application claims the benefit of U.S. Provisional Application No. 63/162,417, filed Mar. 17, 2021, and claims the benefit of U.S. Provisional Application No. 63/051,103, filed Jul. 13, 2020, and claims the benefit of U.S. Provisional Application No. 63/032,815, filed Jun. 1, 2020, the disclosures of which are herein incorporated by reference in their entirety.

### GOVERNMENT SUPPORT

This invention was made with government support under A1131254 awarded by the National Institutes of Health. The government has certain rights in the invention.

### FIELD OF THE TECHNOLOGY

The present disclosure generally relates to the field of biotechnology and medicine and, more particularly, to nucleic acid constructs, polypeptides and vectors that can be used in vaccines for enhanced therapy against respiratory viral infections and methods of use thereof.

### REFERENCE TO SEQUENCE LISTING

This application contains a Sequence Listing that has been submitted in ASCII format via EFS-Web and is hereby incorporated by reference in its entirety. The ASCII copy, created on Jun. 1, 2021, is named 688425_Sequence_listing_ST25.txt, and is 212,366 bytes in size.

### BACKGROUND

Viral infections are responsible for hundreds of thousands of deaths each year. However, treatment options are limited for many viruses. Additionally, carriers of a virus may be asymptomatic, leading to high transmission rates from infected but asymptomatic individuals. Severe acute respiratory syndrome coronavirus 2 (SARS-CoV-2) is the etiologic agent of the Coronavirus Disease 2019 (COVID-19) syndrome, which can rapidly progress to pneumonia, respiratory failure, and systemic inflammatory disease. SARS-CoV-2 is a positive-sense single-stranded RNA virus that was first isolated in late 2019 from patients with severe respiratory illness in Wuhan, China. As a betacoronavirus, SARS-CoV-2 is related to two other highly pathogenic respiratory viruses, SARS-CoV and Middle East respiratory syndrome coronavirus (MERS-CoV). SARS-CoV-2 infection results in a clinical syndrome that can progress to respiratory failure and also present with cardiac pathology, gastrointestinal disease, coagulopathy, and a hyperinflammatory syndrome. The elderly, immunocompromised, and those with co-morbidities (e.g., obesity, diabetes, and hypertension) are at greatest risk of death from COVID-19. More than 152 million infections and 3.2 million deaths have been recorded worldwide since the start of the pandemic.

The extensive morbidity, mortality, and destabilizing socioeconomic consequences of COVID-19 highlight the urgent need for deployment of an effective SARS-CoV-2 vaccine to mitigate the severity of infection, curb transmission, end the pandemic, and prevent its return.

### SUMMARY

Among the various aspects of the present disclosure provide compositions comprising an effective amount of a nanoparticle composition disclosed herein and methods of use thereof.

An aspect of the present disclosure provides for a composition comprising an adenoviral vector having at least a portion of the genome of an adenovirus, wherein the genome of the adenovirus has been modified such that the vector lacks at least the native E1 locus and optionally, the E3 or E3B locus; and/or comprises a betacoronavirus transgene. In one aspect, the transgene is at least a portion of a SARS-CoV-2 spike (S) protein (e.g., Wuhan, South Africa, other variants or mutants thereof, etc.), an immunogenic fragment of the S protein, an immunogenic mutant (e.g., a pre-fusion stabilized mutant, SARS-CoV-2 variant comprising the D614G mutation) of the S protein, or an immunogenic portion, variant, mutant, or fragment thereof (e.g., addition, insertion, deletion, substitution).

Another aspect of the present disclosure provides for a composition comprising a nucleic acid molecule comprising a Simian adenoviral vector; or a nucleic acid encoding the spike protein of a SARS-CoV-2 virus polypeptide or an immunogenic portion, variant, mutant, or fragment thereof. For example, the spike protein can be Wuhan or South Africa or a variant thereof.

Another aspect of the present disclosure provides for a composition comprising a nucleic acid molecule encoding a Simian adenoviral vector; or a nucleic acid that encodes an antigen comprising at least a portion of the SARS-CoV-2 spike (S) protein, an immunogenic fragment of the S protein, an immunogenic mutant (e.g., a pre-fusion stabilized mutant, SARS-CoV-2 variant comprising the D614G mutation) of the S protein, or an immunogenic portion, variant, mutant, or fragment thereof (e.g., addition, insertion, deletion, substitution).

Another aspect of the present disclosure provides for a composition comprising an adenoviral vector encoding a plurality of adenoviral structural proteins or a coronavirus polypeptide selected from the group consisting of a spike protein of a coronavirus or an immunogenic portion, variant, mutant, or fragment thereof.

Another aspect of the present disclosure provides for a composition comprising a recombinant adenovirus backbone comprising in its genome a nucleic acid sequence encoding at least a portion of a coronavirus spike protein (S) protein or stabilized mutant S protein or immunogenic fragment, mutant, or variant thereof.

Another aspect of the present disclosure provides for a composition comprising a coronavirus vaccine comprising: the adenoviral vector of any one of the preceding aspects or embodiments; the nucleic acid encoding a coronavirus polypeptide or an immunogenic portion or variant thereof of any one of the preceding aspects or embodiments; or an excipient or an adjuvant.

In some embodiments, the coronavirus polypeptide or an immunogenic portion is a SARS-CoV-2 spike protein polypeptide or an immunogenic portion, variant, or mutation thereof, such as the SARS-CoV-2 variant comprising the D614G mutation.

In some embodiments, the nucleic acid (or nucleotide) sequence encodes at least an immunogenic portion of a coronavirus spike protein (S) protein, or an immunogenic fragment or variant thereof, substantially replaces the endogenous adenoviral E1 gene in the adenovirus genome.

In some embodiments, the nucleic acid encodes the at least a portion or immunogenic fragment of a coronavirus spike protein (S) protein or immunogenic fragment or variant thereof having a sequence at least 80% identical to the coronavirus spike protein portion of SEQ ID NO: 3; SEQ ID NO: 3 having K986P and V987P mutations; or SEQ ID NO: 3 having a D614G mutation.

In some embodiments, the nucleic acid is at least 80% identical to SEQ ID NO: 5 or SEQ ID NO: 6 and encodes an immunogenic spike protein or immunogenic portion or variant thereof.

In some embodiments, the adenoviral vector is a Simian Ad36 vector (ChAd).

In some embodiments, the coronavirus protein has at least 80% sequence identity with the sequence set forth as SEQ ID NO: 3; SEQ ID NO: 3 having K986P and V987P mutations; or SEQ ID NO: 3 having a D614G mutation.

In some embodiments, the coronavirus protein has at least 85% sequence identity with the sequence set forth as SEQ ID NO: 3; SEQ ID NO: 3 having K986P and V987P mutations; or SEQ ID NO: 3 having a D614G mutation.

In some embodiments, the coronavirus protein has at least 90% sequence identity with the sequence set forth as SEQ ID NO: 3; SEQ ID NO: 3 having K986P and V987P mutations; or SEQ ID NO: 3 having a D614G mutation.

In some embodiments, the coronavirus protein has at least 95% sequence identity with the sequence set forth as SEQ ID NO: 3; SEQ ID NO: 3 having K986P and V987P mutations; or SEQ ID NO: 3 having a D614G mutation.

In some embodiments, the coronavirus protein has at least 99% sequence identity with the sequence set forth as SEQ ID NO: 3; SEQ ID NO: 3 having K986P and V987P mutations; or SEQ ID NO: 3 having a D614G mutation.

In some embodiments, the coronavirus protein has the sequence set forth as SEQ ID NO: 3; SEQ ID NO: 3 having K986P and V987P mutations; or SEQ ID NO: 3 having a D614G mutation.

In some embodiments, the antigen comprises mutations that stabilize spike in the pre-fusion trimer.

In some embodiments, the antigen is a portion or stabilized portion of the SARS-CoV-2 spike (S) surface glycoprotein, a sequence substantially identical thereto, or a functional fragment or functional mutation or variant thereof.

In some embodiments, the antigen is at least a functional portion or mutant of the S protein having two amino acid mutations, K986P and V987P of SEQ ID NO: 3.

In some embodiments, the antigen is at least a functional portion or mutant of the S protein having a D614G mutation in the sequence according to SEQ ID NO: 3.

In some embodiments, the adenovirus is a non-human adenovirus.

In some embodiments, the adenovirus is a simian adenovirus.

In some embodiments, the adenovirus is SAd36.

In some embodiments, the adenovirus is a simian adenovirus, SAd36, having deletions in the E1 and E3B genes (SEQ ID NO: 4; nucleotides 30072-31869 and nucleotides 455-3026, respectively).

In some embodiments, the adenovirus lacks a functional E1 locus and E3 locus.

An aspect of the present disclosure provides for an immunogenic composition comprising the composition according to any one of the preceding aspects or embodiments and optionally one or more additional active ingredients, a pharmaceutically acceptable carrier, diluent, excipient or adjuvant.

An aspect of the present disclosure provides for a polynucleotide sequence encoding the composition or adenoviral vector of any one of the preceding aspects or embodiments.

An aspect of the present disclosure provides for a host cell transduced with the composition or adenoviral vector of any one of the preceding aspects or embodiments.

An aspect of the present disclosure provides for a packaging cell line producing the composition or viral vector of any one of the preceding aspects or embodiments.

In some embodiments, the cell comprises the complement of any viral genes (e.g., E1) functionally deleted from the viral vector of any one of the preceding aspects or embodiments, thereby allowing viral replication through complementation.

An aspect of the present disclosure provides for a kit comprising: (i) the host cell, the cell line, the composition, or the adenoviral vector according to any one of the preceding aspects or embodiments or an immunogenic composition thereof, or (ii) instructions for use.

An aspect of the present disclosure provides for a recombinant adenovirus vector comprising in its genome a nucleic acid sequence encoding at least a portion of a coronavirus spike protein (S) protein or immunogenic fragment or variant thereof (e.g., SEQ ID NO: 3 having K986P and V987P mutations, SEQ ID NO: 3 having a D614G mutation).

In some embodiments, the nucleic acid or nucleotide sequence encodes at least an immunogenic portion of a coronavirus spike protein (S) protein, or an immunogenic fragment or variant/mutant thereof, substantially replaces the endogenous adenoviral E1 gene in the adenovirus genome.

In some embodiments, the nucleic acid encoding the at least a portion or immunogenic fragment of a coronavirus spike protein (S) protein or immunogenic fragment or variant/mutant thereof has a sequence at least 80% identical to the coronavirus spike protein portion of SEQ ID NO: 3; SEQ ID NO: 3 having K986P and V987P mutations; or SEQ ID NO: 3 having a D614G mutation.

An aspect of the present disclosure provides for a pharmaceutical composition comprising the recombinant adenovirus vector of any one of the preceding aspects or embodiments, and a pharmaceutically acceptable carrier.

In some embodiments, the composition further comprises an adjuvant.

An aspect of the present disclosure provides for a coronavirus vaccine comprising the composition or recombinant adenovirus vector of any one of the preceding aspects or embodiments.

An aspect of the present disclosure provides for a nucleic acid encoding the recombinant adenovirus vector according to any one of the preceding aspects or embodiments.

In some embodiments, the nucleic acid has a sequence that is at least 80% identical to the portion encoding the coronavirus spike protein of SEQ ID NO: 3; SEQ ID NO: 3 having K986P and V987P mutations; or SEQ ID NO: 3 having a D614G mutation.

An aspect of the present disclosure provides for an expression vector comprising the nucleic acid of any one of the preceding aspects or embodiments.

An aspect of the present disclosure provides for a cell comprising the nucleic acid or the expression vector according to any one of the preceding aspects or embodiments.

An aspect of the present disclosure provides for a composition comprising the sera of a subject previously administered the composition of any one of the preceding aspects or embodiments.

An aspect of the present disclosure provides for a method of treating a second subject having a coronavirus infection comprising administering to the subject an immunogenically effective amount of the composition comprising sera according to the preceding aspect.

An aspect of the present disclosure provides for a method for inducing an immune response against coronavirus in a subject, comprising administering to the subject an immunogenically effective amount of a composition or vaccine according to any one of the preceding aspects or embodiments.

An aspect of the present disclosure provides for a method of treating or preventing a coronavirus infection in a subject, comprising administering to the subject an immunogenically effective amount of a composition or vaccine of any one of the preceding aspects or embodiments.

An aspect of the present disclosure provides for a method for protecting a subject from coronavirus, comprising administering to the subject an immunogenically effective amount of a composition or vaccine according to any one of preceding aspects or embodiments.

In some embodiments, the immunogenically effective amount of a composition or vaccine protects against SARS-CoV-2 infection in the nasal passages, upper airways, lung tissues, and all other sites of dissemination and/or prevents upper respiratory tract infection and nasal virus shedding.

In some embodiments, the immunogenically effective amount of a composition or vaccine is administered intranasally.

In some embodiments, the immunogenically effective amount of a composition or vaccine is administered intramuscularly.

In some embodiments, the coronavirus is a SARS-CoV-2 virus, SARS-CoV-2 variant comprising the D614G mutation, or a SARS-CoV-2 mutant.

In some embodiments, the subject is a human.

In some embodiments, the subject has been exposed to a coronavirus.

In some embodiments, the subject does not have, but is at risk of developing a coronavirus infection.

In some embodiments, the subject is traveling to a region where the coronavirus is prevalent.

In some embodiments, the subject is exposed to coronavirus.

In some embodiments, delivering the vaccine results in an antigen-specific immune response.

In some embodiments, the subject has, is suspected of having, or is at risk for developing a coronavirus infection.

In some embodiments, the method comprises delivering a transgene into a host cell of the subject.

An aspect of the present disclosure provides for a method of making a recombinant adenovirus comprising: transfecting a cell with a plasmid encoding a recombinant adenovirus vector comprising a SARS-CoV-2 spike protein, mutant, or stabilized mutant thereof; culturing the cell under conditions such that the cell produces the recombinant adenovirus; and collecting the recombinant adenovirus.

In some embodiments, the cells are HEK cells, Vero, or PER cells.

An aspect of the present disclosure provides for a method of producing a recombinant adenoviral vector, comprising the step of incorporating a polynucleotide sequence encoding said adenoviral vector into a host cell, wherein the adenoviral vector is capable of producing an adenovirus-stabilized spike protein, an adenovirus wild-type spike protein or an immunogenic fragment or variant/mutant thereof.

While multiple embodiments are disclosed, still other embodiments of the present disclosure will become apparent to those skilled in the art from the following detailed description, which shows and describes illustrative embodiments of the invention.

### BRIEF DESCRIPTION OF THE FIGURES

Those of skill in the art will understand that the drawings, described below, are for illustrative purposes only. The drawings are not intended to limit the scope of the present teachings in any way.

The application file contains at least one drawing executed in color. Copies of this patent application publication with color drawing(s) will be provided by the Office upon request and payment of the necessary fee.

FIG. 1A-1H depict Immunogenicity of ChAd-SARS-CoV-2-S. FIG. 1A shows a diagram of transgene cassettes: ChAd-control has no transgene insert; ChAd-SARS-CoV-2-S encodes for SARS-CoV-2 S protein with the two indicated proline mutations. FIG. 1B shows binding of ChAd-SARS-CoV-2-S transduced 293 cells with anti-S mAbs. (Left) Summary: +, ++, +++, − indicate <25%, 25-50%, >50%, and no binding, respectively. (Right) Representative flow cytometry histograms of two experiments. FIG. 1C shows four-week old female BALB/c mice were immunized via intramuscular route with ChAd-control or ChAd-SARS-CoV-2-S and boosted four weeks later. Antibody responses in sera of immunized mice at day 21 after priming or boosting were evaluated. FIG. 1D shows ELISA measured anti-S and RBD IgG levels. FIG. 1E shows FRNT determined neutralization activity. Data are pooled from two experiments (n=15 to 30; Mann-Whitney test: ****, P<0.0001). FIG. 1F shows cell-mediated responses were analyzed at day 7 post-booster immunization after re-stimulation with an S protein peptide pool. Splenocytes were assayed for IFNγ and granzyme B expression in CD8+ T cells and granzyme B only in CD4⁺ T cells by flow cytometry. FIG. 1G shows a summary of frequencies and numbers of positive cell populations (n=5; Mann-Whitney test: *, P<0.05; **, P<0.01; ***, P<0.001). Bars indicate median values, and dotted lines are the limit of detection (LOD) of the assays. FIG. 1H shows spleens were harvested at 7 days post-boost, and SARS-CoV-2 spike-specific IgG+ antibody-secreting cells (ASC) frequency was measured by ELISPOT (Mann-Whitney test: ****, P<0.0001). Bars and columns show median values, and dotted lines indicate the limit of detection (LOD) of the assays.

FIG. 2A-2D depict ChAd-SARS-CoV-2-S vaccine induces neutralizing antibodies as measured by FRNT. Four-week old female BALB/c mice were primed or primed and boosted with ChAd-control or ChAd-SARS-CoV-2-S via intramuscular route. FIG. 2A shows serum from ChAd-control immunized mice collected at day 21 after priming or boosting (as described in FIG. 1) was assayed for S-specific IgG responses by ELISA. FIG. 2B shows serum samples from ChAd-control or ChAd-SARS-CoV-2 vaccinated mice were collected at day 21 after priming. FIG. 2C shows serum samples from ChAd-control or ChAd-SARS-CoV-2 vaccinated mice were collected at day 21 after boosting and assayed for neutralizing activity by FRNT. Serum neutralization curves corresponding to individual mice are shown for the indicated vaccines (n=15-30 per group). Each point represents the mean of two technical replicates with error bars denoting the standard deviations (SD). FIG. 2D shows an ELISA measured anti-SARS-CoV-2 NP IgG responses in paired sera obtained 5 days before and 8 days after SARS-CoV-2 challenge of ChAd-control or ChAd-SARS-CoV-2-S mice vaccinated by an intramuscular route (n=5: ** P<0.01; *** P<0.001; paired t test). Dotted lines represent the mean IgG titers from naïve sera.

FIG. 3 shows Gating strategy for analyzing T cell responses. Four-week old female BALB/c mice were immunized with ChAd-control or ChAd-SARS-CoV-2-S and boosted four weeks later. T cell responses were analyzed in splenocytes at day 7 post-boost. Cells were gated for lymphocytes (FSC-A/SSC-A), singlets (SSC-W/SSC-H), live cells (Aqua-), CD45+, CD19− followed by CD4+ or CD8+ cell populations expressing IFNγ or granzyme B.

FIG. 4A-4B depict the impact of pre-existing ChAd immunity on transduction of mice with Hu-AdV5-hACE2. Four-week old female BALB/c mice were primed or primed and boosted. Serum samples were collected one day prior to Hu-AdV5-hACE2 transduction. FIG. 4A shows neutralizing activity of Hu-AdV5-hACE2 in the sera from the indicated vaccine groups was determined by FRNT after prime only.

FIG. 4B shows neutralizing activity of Hu-AdV5-hACE2 in the sera from the indicated vaccine groups was determined by FRNT after prime and boost. Each symbol represents a single animal; each point represents two technical repeats and bars indicate the range. A positive control (anti-Hu-Adv5 serum) is included as a frame of reference.

FIG. 5A-5G show Protective efficacy of intramuscularly delivered ChAd-SARS-CoV-2-S against SARS-CoV-2 infection. FIG. 5A is a scheme of vaccination and challenge. Four-week-old BALB/c female mice were immunized ChAd-control or ChAd-SARS-CoV-2-S. Some mice received a booster dose of the homologous vaccine. On day 35 post-immunization, mice were challenged with SARS-CoV-2 as follows: animals were treated with anti-Ifnar1 mAb and transduced with Hu-AdV5-hACE2 via an intranasal route one day later. Five days later, mice were challenged with 4×10⁵ focus-forming units (FFU) of SARS-CoV-2 via the intranasal route. FIG. 5B shows tissues were harvested at 4 and 8 dpi for analysis. Infectious virus in the lung was measured by plaque assay. FIG. 5C shows viral RNA levels were measured in the lung, spleen and heart at 4 and 8 dpi by RT-qPCR (C) (n=3-7, Mann-Whitney test: *** P<0.001). FIG. 5D shows viral RNA in situ hybridization using SARS-CoV-2 probe (brown color) in the lungs harvested at 4 dpi. Images show low-(top; scale bars, 100 μm) and medium-(middle; scale bars, 100 μm) power magnification with a high-power magnification inset (representative images from n=3 per group). FIG. 5E shows fold change in gene expression of indicated cytokines and chemokines from lung homogenates at 4 dpi was determined by RT-qPCR after normalization to Gapdh levels and comparison with naïve unvaccinated, unchallenged controls (n=7; Mann-Whitney test: ***, P<0.001). FIG. 5F show mice that received a prime-boost immunization were challenged on day 35 post-booster immunization. Tissues were collected at 4 dpi for analysis. Infectious virus in the lung was determined by plaque assay. FIG. 5G shows mice that received a prime-boost immunization were challenged on day 35 post-booster immunization and viral RNA was measured in the lung, spleen and heart using RT-qPCR (G) (n=6-7; Mann-Whitney test: **, P<0.01). (B-C and E-G) Columns show median values, and dotted lines indicate the LOD of the assays.

FIG. 6 show single-dose intramuscular vaccination with ChAd-SARS-CoV-2-S protects mice against SARS-CoV-2-induced inflammation in the lung. Four-week old female BALB/c mice were immunized with ChAd-control and ChAd-SARS-CoV-2-S and challenged following the scheme described in FIG. 5. Lungs were harvested at 8 dpi. Sections were stained with hematoxylin and eosin and imaged at 40× (left; scale bar, 250 μm), 200× (middle; scale, 50 μm), and 400× (right; scale bar, 25 μm) magnifications. Each image is representative of a group of 3 mice.

FIG. 7A-7J show immune responses after Intranasal immunization of ChAd-SARS-CoV-2-S. FIG. 7A shows a scheme of experiments. Five-week-old BALB/c female mice were immunized with ChAd-control or ChAd-SARS-CoV-2-S via an intranasal route. FIG. 7B shows antibody responses in sera of immunized mice at one month after priming were evaluated. An ELISA measured SARS-CoV-2 S- and RBD-specific IgG. FIG. 7C shows an ELISA measured SARS-CoV-2 S- and RBD-specific IgA levels. FIG. 7D shows a FRNT determined neutralization activity. Data are pooled from two experiments with n=10-25 mice per group (Mann-Whitney test: ****, P<0.0001).

FIG. 7E shows mice that received a booster dose were sacrificed one week later to evaluate mucosal and cell-mediated immune responses. SARS-CoV-2 S- and RBD-specific IgG. FIG. 7F shows SARS-CoV-2 S- and RBD-specific IgA levels in BAL fluid were determined by ELISA. FIG. 7G shows neutralizing activity of BAL fluid against SARS-CoV-2 was measured by FRNT. FIG. 7H shows CD8+ T cells in the lung were assayed for IFNγ and granzyme B expression by flow cytometry after re-stimulation with an S protein peptide pool. FIG. 7I shows CD8+ T cells in the lung also were phenotyped for expression of CD103 and CD69. FIG. 7J shows SARS-CoV-2 spike-specific IgG+ and IgA+ antibody-secreting cells (ASC) frequency in the spleen harvested one week post-boost was measured by ELISPOT. Data for mucosal and cell-mediated responses are pooled from two experiments (E-I: n=7-9 per group; Mann-Whitney test: ***, P<0.001); J: n=5 per group; Mann-Whitney test: **, P<0.01; ***, P<0.001). (B-J) Bars and columns show median values, and dotted lines indicate the LOD of the assays.

FIG. 8A-8C show intranasal inoculation of ChAd-SARS-CoV-2-S induces neutralizing antibodies as measured by FRNT. Five-week old female BALB/c mice were immunized with ChAd-control or ChAd-SARS-CoV-2-S via an intranasal inoculation route. Serum samples collected one month after immunization were assayed for neutralizing activity by FRNT. Mice were boosted at day 30 after priming and were sacrificed one week later to evaluate immune responses. FIG. 8A shows serum samples from ChAd-control or ChAd-SARS-CoV-2-S vaccinated mice were tested for neutralizing activity with SARS-CoV-2 strain 2019 n-CoV/USA_WA1/2020 (n=8-10 per group). FIG. 8B shows serum samples from ChAd-SARS-CoV-2-S vaccinated mice were tested for neutralization of recombinant luciferase-expressing SARS-CoV-2 viruses (wild-type (left) and D614G variant (middle)). (Right) Paired EC50 values are indicated (n=5; n.s. not significant, paired t test). FIG. 8C shows BAL fluid was collected from ChAd-control or ChAd-SARS-CoV-2-S vaccinated mice, and neutralization of SARS-CoV-2 strain 2019 n-CoV/USA_WA1/2020 was measured using a FRNT assay (n=8-10 per group). Each point represents the mean of two technical replicates with error bars denoting the SD.

FIG. 9A-9E show Single-dose intranasal immunization with ChAd-SARS-CoV-2-S protects against SARS-CoV-2 infection. Five-week-old BALB/c female mice were immunized with ChAd-control or ChAd-SARS-CoV-2-S via an intranasal route. On day 35 post-immunization, mice were challenged as follows: animals were treated with anti-Ifnar1 mAb and transduced with Hu-AdV5-hACE2 via the intranasal route one day later. Five days later, mice were challenged intranasally with 4×105 FFU of SARS-CoV-2. FIG. 9A shows tissues and nasal washes were collected at 4 and 8 dpi for analysis. Infectious virus in the lung was measured by plaque assay. FIG. 9B shows viral RNA levels in the lung, spleen, heart, nasal turbinates, and nasal washes were measured at 4 and 8 dpi by RT-qPCR. FIG. 9C shows fold change in gene expression of indicated cytokines and chemokines was determined by RT-qPCR, normalized to Gapdh, and compared to naïve controls in lung homogenates at 4 dpi (2 experiments, n=6-9; median values are shown: *, P<0.05, ** P<0.01, *** P<0.001, **** P<0.0001; Mann-Whitney test). Columns show median values, and dotted lines indicate the LOD of the assays. FIG. 9D shows lungs were harvested at 8 dpi. Sections were stained with hematoxylin and eosin and imaged at 40× (left; scale bar, 250 μm), 200× (middle; scale, 50 μm), and 400× (right; scale bar, 25 μm) magnifications. Each image is representative of a group of 3 mice. FIG. 9E show an ELISA measured anti-SARS-CoV-2 NP IgM (left) and IgG (right) antibody responses in paired sera obtained 5 days before and 8 days after SARS-CoV-2 challenge of ChAd-control or ChAd-SARS-CoV-2-S mice vaccinated by an intranasal route (n=6: ns; not significant; ** P<0.01, **** P<0.0001; paired t test). Dotted lines represent the mean IgM and IgG titers from naïve sera (n=6).

FIG. 10A-10N show ChAd-SARS-CoV-2-S induces durable immunity. FIG. 10A shows immunization scheme. Five-week old female BALB/c mice were vaccinated via IN or IM route with 10¹⁰ viral particles of ChAd-control or decreasing doses (10¹⁰, 10⁹ and 10⁸ vp) of ChAd-SARS-CoV-2-S. FIG. 10B-10M show humoral responses in sera of immunized mice were evaluated (n=6-14). An ELISA measured anti-S and RBD IgG and IgA levels from IN-immunized mice at 100 (FIG. 10B and FIG. 10C) or 200 (FIG. 10H-10I) days post-vaccination, or from IM-immunized mice at 100 (FIG. 10E-10F) or 200 (FIG. 10K-10L) days post-vaccination. Neutralizing activity of sera determined by FRNT from IN-(FIG. 10D, FIG. 10J) or IM-(FIG. 10G, FIG. 10M) immunized mice at 100 (FIG. 10D, FIG. 10G) or 200 (FIG. 10J, FIG. 10M) days post-vaccination. FIG. 10N shows the frequency of S-specific IgG or IgA producing LLPCs in the bone marrow measured by ELISPOT assay (n=4). For (FIG. 10B-10M): one way ANOVA with a Dunnett's post-test comparing vaccine and control groups: ns, not significant; **, P<0.01; ****, P<0.0001). For (FIG. 10N): Mann-Whitney test: *, P<0.05. B-N, Bars show median values, and dotted lines indicate the limit of detection (LOD) of the assays.

FIG. 11A-11B show ChAd-SARS-CoV-2-S vaccine induces neutralizing antibodies as measured by FRNT. Five-week old female BALB/c mice were immunized via IN or IM route with a single 10¹⁰, 10⁹, or 10⁸ dose of ChAd-SARS-CoV-2-S. FIG. 11A shows serum samples from ChAd-SARS-CoV-2-S vaccinated mice were collected at day 100. FIG. 11B shows serum samples from ChAd-SARS-CoV-2-S vaccinated mice were collected at day 200 after immunization and assayed for neutralizing activity by FRNT. Serum neutralization curves corresponding to individual mice are shown for the indicated vaccines (n=6-14 per group). Each point represents the mean of two technical replicates.

FIG. 12A-12E show intranasal inoculation of ChAd-SARS-CoV-2-S induces antibody responses with Fc effector function capacity. FIG. 12A shows serum was analyzed by Luminex platform to quantify the amount of anti-SARS-CoV-2 (WA1/2020 D614G) spike and RBD IgG1. Bars represent the mean values. FIG. 12B shows serum was analyzed by luminex to quantify the amount of anti-SARS-CoV-2 IgG1 to different SARS-CoV-2 protein variants. Polar plots represent the IgG1 median percentile rank for each SARS-CoV-2 protein and variant. FIG. 12C shows a heatmap shows the IgG titer and FcγR binding titer of each vaccine regimen to SARS-CoV-2 Spike or RBD proteins. Each square represents the average z-score within a group for the condition. FIG. 12D shows serum was incubated with primary mouse neutrophils (mADNP) or J774A.1 cells (mADCP) and SARS-CoV-2 spike-coated beads, and phagocytosis was measured after 1 h. Bars represent the mean and the error bars indicate standard deviations. FIG. 12E shows serum was incubated with primary mouse neutrophils (mADNP) or J774A.1 cells (mADCP) and WA1/2020 D614G, B.1.1.7, or B1.351 spike-coated beads, and phagocytosis was measured after 1 h. Polar plots represent the mADNP or mADCP median percentile rank for each SARS-CoV-2 protein and variant. For (A and D): one-way ANOVA with a Dunnett's post-test comparing vaccine to control groups: **, P<0.01; ***, P<0.001; ****, P<0.0001). (A and D): Bars indicate median values.

FIG. 13A-13L show durability of protective efficacy of ChAd-SARS-CoV-2-S against SARS-CoV-2 infection in BALB/c mice. Five-week old female BALB/c mice were immunized via IN or IM route with 10¹⁰ vp of ChAd-control or 10¹⁰, 10⁹ and 10⁸ vp of ChAd-SARS-CoV-2-S. On day 100 or 200 post-immunization, mice were challenged as follows: animals were treated with anti-Ifnar1 mAb and transduced with Hu-AdV5-hACE2 via an IN route one day later. Five days later, mice were inoculated with 5×104 FFU of SARS-CoV-2 WA1/2020 via the intranasal route. Tissues were harvested at 4 dpi, and viral RNA levels were measured from mice challenged 100 (FIG. 13A-13F) or 200 (FIG. 13G-13L) days post-immunization by RT-qPCR (n=6-14, Kruskal Wallis with Dunn's post-test: ns, not significant; **, P<0.01; *, P<0.1; ***, P<0.001 ****, P<0.0001). Bars show median values, and dotted lines indicate the LOD of the assays.

FIG. 14A-14H show immunogenicity of intranasally administration ChAd-SARS-CoV-2-S in K18-hACE2 mice. Five-week-old K18-hACE2 female mice were immunized with 10⁹ vp ChAd-control or ChAd-SARS-CoV-2-S via an IN route. Antibody responses in sera of mice at 6 weeks (FIG. 14A-14D, n=20) or 9 months (FIG. 14E-14H, n=7) after immunization were evaluated. An ELISA measured SARS-CoV-2 S- and RBD-specific IgG (FIG. 14A, FIG. 14E) and IgA levels (FIG. 14B, FIG. 14F), and an FRNT determined neutralization activity (FIG. 14C, FIG. 14D, FIG. 14G, FIG. 14H). FIG. 14C-14D, FIG. 14G-14H, Paired analysis of serum neutralizing activity from immunized mice collected at 6 weeks (FIG. 14C, FIG. 14D) or 9 months (FIG. 14G) against WA1/2020 and Wash-B.1.351 (FIG. 14C, FIG. 14G), or Wash-B.1.1.28 (FIG. 14D, FIG. 14H). FIG. 14A-14B, FIG. 14E-14F: Mann-Whitney test: ***, P<0.001; ****, P<0.0001. FIG. 14C-14D, FIG. 14G-14H: Two-tailed Wilcoxon matched-pairs signed rank test: *, P<0.05; ****, P<0.0001.

FIG. 15A-15T show ChAd-SARS-CoV-2-S confers cross-protection against variant viruses in K18-hACE2 mice. FIG. 15A shows the experimental scheme. Five-week-old K18-hACE2 female mice were immunized via an IN route with 10¹⁰ vp of ChAd-control or ChAd-SARS-CoV-2-S. FIG. 15B-15O show at 6 weeks post-immunization, mice were challenged with 104 FFU of SARS-CoV-2 of Wash-B.1.351 (FIG. 15B-15F), Wash-B.1.1.28 (FIG. 15G-15K), or WA1/2020 (FIG. 15L-15O). FIG. 15P-15T show at 9 months post-immunization, mice were challenged with 104 FFU of Wash-B.1.351. FIG. 15B, FIG. 15G, FIG. 15L, FIG. 15P. Body weight change overtime. Data are the mean±SEM comparing vaccine to control groups (n=6-9 for each group; unpaired t test for area under curve, **** P<0.0001). FIG. 15C-15F, FIG. 15H-15K, FIG. 15M-15O, FIG. 15Q-15T. Viral RNA levels in the lung, heart, brain, and nasal washes were measured at 6 dpi by RT-qPCR (n=6-9; Mann-Whitney test: ** P<0.01, *** P<0.001). Bars show median values, and dotted lines indicate the LOD of the assays.

FIG. 16A-16B show ChAd-SARS-CoV-2-S vaccine induces neutralizing antibodies as measured by FRNT. Related to FIG. 4. Five-week-old K18-hACE2 female mice were immunized with 109 vp ChAd-control or ChAd-SARS-CoV-2-S via an IN route. FIG. 16A shows serum samples were collected at six weeks. FIG. 16B shows serum samples collected at nine months after immunization and assayed for neutralizing activity against WA1/2020, Wash-B.1.351, or Wash-B.1.1.28 by FRNT. Serum neutralization curves corresponding to individual mice are shown for the indicated vaccines (n=7-20 per group). Each point represents the mean of two technical replicates.

### DETAILED DESCRIPTION

The present disclosure is based, at least in part, on the development of recombinant non-human adenoviral vector compositions and immunogenic compositions thereof for treating or preventing coronavirus infection. In addition, the disclosure provides methods of administering the compositions disclosed herein providing durable cellular and humoral mediated immunity against a coronavirus infection. For example, the present disclosure provides compositions for intranasal administration which show improved mucosal immunity induced by intranasal vaccination relative to intramuscular administration thereby limiting SARS-CoV-2 transmission.

As shown herein intramuscular dosing of an adenoviral composition of the present disclosure induces robust systemic humoral and cell-mediated immune responses against the S protein, and protects against lung infection, inflammation, and pathology after SARS-CoV-2 challenge, however, residual viral infection was detected. In contrast, a single intranasal dose of an adenoviral composition of the present disclosure induces high levels of systemic and mucosal IgA immune responses, completely prevents infection in the upper and lower respiratory tracts, and confers sterilizing immunity. Importantly, sera obtained from vaccinated subjects also efficiently neutralizes SARS-CoV-2 variants (e.g., variants containing the D614G mutation in the S protein; viruses with this adaptive mutation are now circulating worldwide and associated with greater infectivity, at least in cell culture). Thus, intranasal administration (even as a single-dose) of an adenoviral composition of the present disclosure is useful for preventing SARS-CoV-2 infection and transmission, and curtailing its pandemic spread.

Applicant has discovered a single-dose intranasal immunization with the disclosed compositions promotes superior humoral immunity than intramuscular immunization; 100-fold lower inoculating doses induce robust neutralizing antibody responses; intranasal but not intramuscular immunization induces serum IgA responses and IgA-specific long-lived plasma cells (LLPCs) against the SARS-CoV-2 S protein; and the humoral immunity induced by the compositions of the disclosure is durable and rises over a six-month period after vaccination.

Severe acute respiratory syndrome coronavirus 1 (SARS-CoV-1) that was responsible for SARS epidemic in 2002-2004, Middle East respiratory syndrome coronavirus (MERS-CoV) that caused MERS first reported in 2012, and SARS-CoV-2 that has been responsible for the more recent coronavirus disease 2019 (Covid-19) pandemic all bind to angiotensin converting enzyme 2 (ACE2) on the surface of the cells in order to infect the cells. Basically ACE-2 is the functional receptor for SARS-CoV-1, SARS-CoV-2, and MERS-COV and most likely future SARS-COV variants. ACE-2 is an important component of Renin-Angiotensin-Aldosterone System (RAAS). ACE-2 converts angiotensin 2 to angiotensin 1-7. High angiotensin 2 is associated with vasoconstriction, inflammation, and acute lung injury. ACE2 is expressed in various organs including lungs, heart, kidney, liver, intestine, and other tissues. SARS-CoV virus bind to ACE-2 and enter the cells.

The SARS-CoV-2 RNA genome is approximately 30,000 nucleotides in length. The 5′ two-thirds encode nonstructural proteins that enable genome replication and viral RNA synthesis. The remaining one-third encode structural proteins such as spike (S), envelope, membrane, and nucleoprotein (NP) that form the spherical virion, and accessory proteins that regulate cellular responses. The S protein forms homotrimeric spikes on the virion and engages the cell-surface receptor angiotensin-converting enzyme 2 (ACE2) to promote coronavirus entry into human cells. The SARS-CoV and SARS-CoV-2 S proteins are cleaved sequentially during the entry process to yield S1 and S2 fragments, followed by further processing of S2 to yield a smaller S2′ protein (Hoffmann et al., 2020). The S1 protein includes the receptor binding domain (RBD) and the S2 protein promotes membrane fusion. The structure of a soluble, stabilized prefusion form of the SARS-CoV-2 S protein was solved by cryo-electron microscopy, revealing considerable similarity to the SARS-CoV S protein. This form of the S protein is recognized by potently neutralizing monoclonal antibodies and could serve as a promising vaccine target.

Release of the SARS-CoV-2 genome sequence prompted academic, government, and industry groups to immediately begin developing vaccine candidates that principally targeted the viral S protein. Multiple platforms have been developed to deliver the SARS-CoV-2 S protein including DNA plasmid, lipid nanoparticle encapsulated mRNA, inactivated virion, and viral-vectored vaccines. Several vaccines have entered clinical trials to evaluate safety, and some have advanced to trials assessing immunogenicity and efficacy. Because of the urgency of the pandemic, most vaccines advanced to human testing without substantive efficacy data in animals. This circumstance occurred in part because vaccine design and development has outpaced the generation of accessible pre-clinical disease models of SARS-CoV-2 infection and pathogenesis.

Adenovirus (Ad)-based vaccines against betacoronaviruses have been evaluated previously. A single dose of a chimpanzee Ad-vectored vaccine encoding the full-length S protein of MERS-CoV protected human dipeptidyl peptidase 4 (hDPP4) transgenic mice from infection, reduced virus shedding and enhanced survival in camels, and was safe and immunogenic in humans in a phase 1 clinical trial. A human Ad-based vaccine expressing a MERS S1-CD40L fusion protein also was protective in transgenic hDPP4 mice. An Ad-based SARS-CoV vaccine expressing a full-length S protein prevented pneumonia in ferrets after challenge and was highly immunogenic in rhesus macaques. A chimpanzee Ad vector (Y25, a simian Ad-23) encoding the wild-type SARS-CoV-2 S protein (ChAdOx1 nCoV-19) is currently under evaluation in humans as a single intramuscular injection (NCT04324606). Preliminary pre-print analysis suggests this vaccine protects against lung infection and pneumonia but not against upper respiratory tract infection and nasal virus shedding (doi.org/10.1101/2020.05.13.093195).

Disclosed herein are compositions, methods, and treatment plans for treating an individual who is at risk of having a respiratory viral infection, has mild symptoms of a respiratory viral infection, or has severe symptoms of a respiratory viral infection. A composition of the present disclosure may be used to treat, prevent, or reduce the infectivity of a respiratory viral infection. A treatment plan may comprise administering a composition of the disclosure to an individual at risk of having a viral infection or who has a viral infection, thereby preventing or treating the viral infection. In some embodiments, a viral transmission may be prevented or reduced by reducing viral infection in the upper respiratory tract. Compositions and methods of the disclosure provide robust antigen-specific antibody, neutralizing antibody, and B and T cell responses. This confers protection against infection with marked reductions in viral yield, inflammation, and pathology in the lung. Compositions and methods of the disclosure generates robust mucosal immunity including high levels of neutralizing and anti-RBD IgA and IgG in the serum and lung and SARS-CoV-2 specific resident memory T cells in the lung. The disclosed compositions and methods completely protect against SARS-CoV-2 infection in the nasal passages, upper airways, lung tissues, and all other sites of possible dissemination. Based on measurements of anti-NP and anti-ORF8 responses, a single intranasal dose of the presently disclosed compositions confers sterilizing immunity, which has not been described before with any COVID-19 vaccine, much less with a single dose administration.

A composition of the present disclosure may be formulated for locally, for example intra-nasally (e.g., as a nasal spray, or inhalation), or systemically (e.g., intravenous or intraperitoneal) and administered for treating or preventing a respiratory viral infection (e.g., a coronavirus infection such as SARS-CoV-2). The compositions of the present disclosure (e.g., compositions formulated for nasal delivery or inhalation) may be administered to a subject who may be at risk of contracting a viral infection (e.g., SARS-CoV-2). For example, the compositions of the present disclosure may be administered to individuals in high risk environments (e.g., healthcare workers), individuals who have been or who are suspected to have been exposed to a virus (e.g., SARS-CoV-2), or individuals who have tested positive for a viral infection. A composition of the present disclosure may be administered to an individual who is displaying symptoms of a respiratory infection (e.g., a SARS-CoV-2 infection) or who is asymptomatic at the time of administration. In some embodiments, the compositions of the present disclosure may be self-administered by the individual (e.g., as a nasal spray or inhalation) and may be administered outside of a medical facility (e.g., at home).

The methods and compositions disclosed herein may be used to treat, prevent, or reduce the infectivity of a respiratory viral infection. In some embodiments, the viral infection may be a coronavirus infection. Pathogens with long incubation periods, such as SARS-CoV-2 which has a median incubation period of about five days, may have high risk of transmission since many infected individuals may be unaware that they are infected. Additionally, carriers of coronavirus may frequently be asymptomatic or have mild symptoms, leading to unknowing contact between a viral host and other members of a population. A subject at risk for a coronavirus infection may come in contact with an asymptomatic carrier of the coronavirus infection, thereby unknowingly contracting the coronavirus infection. Methods and compositions are needed to prevent coronavirus infections in at-risk individuals (e.g., individuals who have come in contact with a carrier of a coronavirus or who may come in contact with a carrier of a coronavirus). In some embodiments, the compositions, methods, or treatment regiments disclosed herein may treat or prevent a SARS-CoV-2 infection (e.g., COVID-19).

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Various aspects of the invention are described in further detail in the following sections.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

An adjuvant refers to a vehicle used to enhance antigenicity. In some embodiments, an adjuvant can include a suspension of minerals (alum, aluminum hydroxide, or phosphate) on which antigen is adsorbed; or water—in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1 BBL and toll-like receptor (TLR) agonists, such as TLR-9 agonists. Additional description of adjuvants can be found, for example, in Singh (ed.) Vaccine Adjuvants and Delivery Systems. Wiley-Interscience, 2007). Adjuvants can be used in combination with the disclosed compositions.

An amino acid substitution is the replacement of one amino acid in a polypeptide with a different amino acid.

The term “antibody” refers to an immunoglobulin, antigen-binding fragment, or derivative thereof, which specifically binds and recognizes an analyte (antigen) such as a coronavirus S protein, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

“Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that reduce an activity or function of the recombinant coronavirus S ectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Coronavirus is a family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future.

Non-limiting examples of betacoronaviruses include Middle East respiratory syndrome coronavirus (MERS-CoV), Severe Acute Respiratory Syndrome coronavirus (SARS-CoV), Human coronavirus HKU1 (HKUI-CoV), Human coronavirus OC43 (OC43-CoV), Murine Hepatitis Virus (MHV-CoV), Bat SARS-like coronavirus WIV1 (WIVI-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5-portion of the genome, and structural genes included in the 3-portion of the genome.

Coronavirus Spike (S) protein: A class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate SI and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain.

Coronavirus Spike (S) protein prefusion conformation is a structural conformation adopted by the ectodomain of the coronavirus S protein following processing into a mature coronavirus S protein in the secretory system, and prior to triggering of the fusogenic event that leads to transition of coronavirus S to the postfusion conformation. The three-dimensional structure of an exemplary coronavirus S protein (HKU1-CoV) in a prefusion conformation is disclosed herein and provided in Kirchdoerfer et al., “Pre-fusion structure of a human coronavirus spike protein,” Nature, 531: 118-121, 2016 (incorporated by reference herein).

A coronavirus S ectodomain trimer “stabilized in a prefusion conformation” comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the prefusion conformation to the postfusion conformation). Additionally, stabilization of the coronavirus S ectodomain trimer in the prefusion conformation can include an increase in resistance to denaturation compared to a corresponding native coronavirus S sequence. Methods of determining if a coronavirus S ectodomain trimer is in the prefusion conformation are provided herein, and include (but are not limited to) negative-stain electron microscopy and antibody binding assays using a prefusion-conformation-specific antibody.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

In one example, a desired response is to inhibit or reduce or prevent CoV (such as SARS-CoV-2) infection. The CoV infection does not need to be completely eliminated or reduced or prevented for the method to be effective. For example, administration of an effective amount of the immunogen can induce an immune response that decreases the CoV infection (for example, as measured by infection of cells, or by number or percentage of subjects infected by the CoV) by a desired amount, for example by at least 50%, at least 60%, at least 70%, at least 80%, at least 90%, at least 95%, at least 98%, or even at least 100% (elimination or prevention of detectable CoV infection), as compared to a suitable control. Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on coronavirus S ectodomain, such as a SARS-CoV S ectodomain. Epitopes can be formed both from contiguous amino acids or noncontiguous amino acids juxtaposed by tertiary folding of a protein.

Expression refers to transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences refer to nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “heterologous” refers to originating from a different genetic source. A nucleic acid molecule that is heterologous to a cell originated from a genetic source other than the cell in which it is expressed. In one specific, non-limiting example, a heterologous nucleic acid molecule encoding a recombinant coronavirus S protein is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cells refer to cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

An Immune response is a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

A nucleic acid molecule is a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term “operably linked” refers to a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

A polypeptide is any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

A prime-boost vaccination is an immunotherapy including administration of a first immunogenic composition (the primary vaccine) followed by administration of a second immunogenic composition (the booster vaccine) to a subject to induce an immune response. The priming vaccine and/or the booster vaccine include a vector (such as a viral vector, RNA, or DNA vector) expressing the antigen to which the immune response is directed. The booster vaccine is administered to the subject after the priming vaccine; a suitable time interval between administration of the priming vaccine and the booster vaccine, and examples of such timeframes are disclosed herein. In some embodiments, the priming vaccine, the booster vaccine, or both primer vaccine and the booster vaccine additionally include an adjuvant. In one non-limiting example, the priming vaccine is a DNA-based vaccine (or other vaccine based on gene delivery), and the booster vaccine is a protein subunit or protein nanoparticle based vaccine.

A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant virus is one that includes a genome that includes a recombinant nucleic acid molecule. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity is the similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch,/. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5: 151-3, 1989; Corpet et al, Nuc. Acids Res. 16: 10881-90, 1988; Huang et al. Computer Appls. in the Biosciences 8, 155-65, 1992; and Pearson et al., Meth. Mol. Bio. 24:307-31, 1994. Altschul et al, J. Mol. Biol. 215:403-10, 1990, presents a detailed consideration of sequence alignment methods and homology calculations.

Homologs and variants of a polypeptide (such as a coronavirus S ectodomain) are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

A vaccine is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In a non-limiting example, a vaccine induces an immune response that reduces the severity of the symptoms associated with a coronavirus infection (such as a SARS-CoV or MERS-CoV infection) and/or decreases the viral load compared to a control. In another non-limiting example, a vaccine induces an immune response that reduces and/or prevents a coronavirus infection (such as a SARS-CoV or MERS-CoV infection) compared to a control.

A vector is an entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP) are a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60: 1445-1456; and Hagensee et al. (1994)/. Virol. 68:4503-4505; Vincente, J Invertebr Pathol., 2011; Schneider-Ohrum and Ross, Curr. Top. Microbiol. Immunol, 354: 53073, 2012).

### II. Compositions

A composition of the present disclosure may comprise one or more active agents. In some embodiments, an active agent may be an agent to prevent, treat, or reduce the infectivity of a viral infection. In some embodiments, treating a viral infection may comprise reducing the infectivity and/or transmission of the virus. In some embodiments, preventing a viral infection may comprise reducing the infectivity and/or transmission of the virus. A composition of the present disclosure may comprise an active agent to prevent a viral infection, an active agent to treat a viral infection, an active agent to reduce the infectivity of a viral infection, or a combination thereof. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

The present disclosure relates to non-human adenoviral vector compositions and methods of using an immunogenic composition comprising the adenovirus vector and optionally optionally one or more additional active ingredients, a pharmaceutically acceptable carrier, diluent, excipient or adjuvant to treat or prevent a respiratory viral infection. Applicant discovered that by using a simian adenovirus vector, issues of heterologous vector cross-immunity were overcome that has been seen with human adenovirus vector platforms (PMID:32450106). Applicant has configured into a chimp adenoviral vector expressing a SARS-CoV-2 antigen, such as a stabilized form of the SARS-CoV-2 “S” protein and have shown it can protect against COVID-19 in an animal model of the disease. A SARS-CoV-2 antigen, or an immunogenic portion thereof, when expressed in an adenovirus vector can, upon infection of a human, stimulate an immune response, and thereby confer immunity to COVID-19 disease.

Other aspects of the invention are described in further detail below.

### a) Adenoviral Vector

The present disclosure provides for an adenoviral vector. Adenoviruses are non-enveloped viruses, approximately 90-100 nm in diameter, comprising a nucleocapsid and a linear double stranded DNA genome. The viral nucleocapsid comprises penton and hexon capsomers. A unique fibre is associated with each penton base and aids in the attachment of the virus to the host cell via the Coxsackie-adenovirus receptor on the surface of the host cell. Over 50 serotype strains of adenoviruses have been identified, most of which cause respiratory tract infections, conjunctivitis and gastroentiritus in humans. Rather than integrating into the host genome, adenoviruses normally replicate as episomal elements in the nucleus of the host cell. The genome of adenoviruses comprises 4 early transcriptional units (E1, E2, E3 and E4), which have mainly regulatory functions and prepare the host cell for viral replication. The genome also comprises 5 late transcriptional units (L1, L2, L3, L4 and L5), which encode structural proteins including the penton (L2), the hexon (L3), the scaffolding protein (L4) and the fiber protein (L5), which are under the control of a single promoter. Each extremity of the genome comprises an Inverted Terminal Repeat (ITR) which is necessary for viral replication.

Recombinant adenoviruses were originally developed for gene therapy, but the strong and sustained transgene-specific immune responses elicited by these gene delivery agents prompted their use as vaccine carriers. In addition to being highly immunogenic, adenoviruses offer many other advantages for clinical vaccine development. The adenoviral genome is relatively small (between 26 and 45 kbp), well characterized and easy to manipulate. The deletion of a single transcriptional unit, E1, renders the virus replication-incompetent which increases its predictability and reduces side effects in clinical applications. Recombinant adenoviruses can accommodate relatively large transgenes, in some cases up to 8kb, allowing flexibility in subunit design, and have a relatively broad tropism facilitating transgene delivery to a wide variety of cells and tissues. Importantly for clinical applications, methods for scaled-up production and purification of recombinant adenoviruses to high titre are well established. Thus far, subgroup C serotypes AdHu2 or AdHu5 have predominantly been used as vectors. However, the first generation of vaccine vectors based on the archetypal human adenovirus AdHu5 showed poor efficacy in clinical trials, despite encouraging preclinical data. It was subsequently discovered that a large proportion of human adults harbour significant titres of neutralizing antibodies to common human serotypes such as AdHu2 and AdHu5, as a result of natural infection. Neutralizing antibodies could reduce the potency of viral vector vaccines by blocking viral entry into host cells and hence delivery of the target transgene.

The compositions described herein include vectors that deliver a heterologous molecule to cells, either for therapeutic or vaccine purposes. As used herein, a vector may include any genetic element including, without limitation, naked DNA, a phage, transposon, cosmid, episome, plasmid, or a virus. In some embodiments such vectors contain simian adenovirus DNA (e.g., SAdV-36), and a transgene. By “transgene” is meant the combination of a selected heterologous gene and the other regulatory elements necessary to drive translation, transcription and/or expression of the gene product in a host cell. In several embodiments, the viral vector can include an adenoviral vector that expresses a transgene encoding a coronavirus S protein. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 36, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 36.

Typically, a SAdV-derived adenoviral vector is designed such that the transgene is located in a nucleic acid molecule which contains other adenoviral sequences in the region native to a selected adenoviral gene. The transgene may be inserted into an existing gene region to disrupt the function of that region, if desired. Alternatively, the transgene may be inserted into the site of a partially or fully deleted adenoviral gene. For example, the transgene may be located in the site of such as the site of a functional E1 deletion or functional E3 deletion (or E3B), among others that may be selected. The term “functionally deleted” or “functional deletion” means that a sufficient amount of the gene region is removed or otherwise damaged, e.g., by mutation or modification, so that the gene region is no longer capable of producing functional products of gene expression. If desired, the entire gene region may be removed. In one embodiment, the adenovirus vector useful according to the disclosure is a simian adenovirus, SAd36, having deletions in the E1 and E3B genes. In one aspect, the adenovirus vector has the nucleic acid sequence of SEQ ID NO: 4. In another aspect, the adenoviral vector has the nucleic acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity with SEQ ID NO: 4.

For example, for a production vector useful for generation of a recombinant virus, the vector may contain the transgene and either the 5′ end of the adenoviral genome or the 3′ end of the adenoviral genome, or both the 5′ and 3′ ends of the adenoviral genome. The 5′ end of the adenoviral genome contains the 5′ cis-elements necessary for packaging and replication; i.e., the 5′ inverted terminal repeat (ITR) sequences (which function as origins of replication) and the native 5′ packaging enhancer domains (that contain sequences necessary for packaging linear Ad genomes and enhancer elements for the E1 promoter). The 3′ end of the adenoviral genome includes the 3′ cis-elements (including the ITRs) necessary for packaging and encapsidation. Suitably, a recombinant adenovirus contains both 5′ and 3′ adenoviral cis-elements and the transgene is located between the 5′ and 3′ adenoviral sequences. A simian based adenoviral vector (e.g. SAdV-36) may also contain additional adenoviral sequences.

Suitably, these simian based adenoviral vectors contain one or more adenoviral elements derived from the adenoviral genome. In one embodiment, the vectors contain adenoviral sequences that are derived from a different adenoviral serotype than that which provides the ITRs. As defined herein, a pseudotyped adenovirus refers to an adenovirus in which the capsid protein of the adenovirus is from a different adenovirus than the adenovirus which provides the ITRs. Chimeric or hybrid adenoviruses may be constructed using the adenoviruses described herein using techniques known to those of skill in the art. See, e.g., U.S. Pat. No. 7,291,498.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

In some embodiments, the transgene encodes a SARS-CoV-2 antigen, such as a stabilized form of the SARS-CoV-2 “S” protein. In some embodiments, transgene encodes at least a portion or immunogenic fragment of a coronavirus spike protein (S) protein or an immunogenic fragment or variant thereof having a sequence at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to the coronavirus spike protein portion (or immunogenic portion or variant thereof) of SEQ ID NO: 3; or at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 3 having K986P and V987P mutations; or SEQ ID NO: 3 having a D614G mutation. In some embodiments, the transgene encodes a spike protein of a SARS-CoV-2 variant, in non-limiting examples a spike protein having a D80G, 144del, F157S, L5F, T951, A67V, S477N, 144del, Q677H, A701V, F888L, T791I, T859N, D950H, E484Q, D614G, E484K, N501Y, Δ69-70, L452R, or K417N or RBD E484K mutation relative to SEQ ID NO: 3. In some embodiments, the transgene encodes a spike protein from a WA1/2020, B.1.1.7, B.1.351, B.1.1.28, P.1, B.1.427, B.1.526, B.1.526.1, B.1.525, P.2, B.1.617, B.1.617.1, B.1.617.2, B.1.617.3, B.1.429, or B.1.429 variants. In each of the above embodiments, the spike protein is further modified to be a prefusion stabilized form (e.g. having double proline substitution between residues 1050 to 1069 or between residues 981 to 999).

In addition to the major elements identified above for the trangene, the vector also includes conventional control elements necessary which are operably linked to the transgene in a manner that permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

A great number of expression control sequences, including promoters which are native, constitutive, inducible and/or tissue-specific, are known in the art and may be utilized. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFIa promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. For example, inducible promoters include the zinc-inducible sheep metallothionine (MT) promoter and the dexamethasone (Dex)-inducible mouse mammary tumor virus (MMTV) promoter. Other inducible systems include the T7 polymerase promoter system [WO 98/10088]; the ecdysone insect promoter [No et al, Proc. Natl. Acad. ScL USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)]. Other systems include the FK506 dimer, VP 16 or p65 using castradiol, diphenol murislerone, the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. The effectiveness of some inducible promoters increases over time. In such cases one can enhance the effectiveness of such systems by inserting multiple repressors in tandem, e.g., TetR linked to a TetR by an IRES. Alternatively, one can wait at least 3 days before screening for the desired function. One can enhance expression of desired proteins by known means to enhance the effectiveness of this system. For example, using the Woodchuck Hepatitis Virus Posttranscriptional Regulatory Element (WPRE).

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. Another embodiment of the transgene includes a transgene operably linked to a tissue-specific promoter. For instance, if expression in skeletal muscle is desired, a promoter active in muscle should be used. These include the promoters from genes encoding skeletal p3-actin, myosin light chain 2A, dystrophin, muscle creatine kinase, as well as synthetic muscle promoters with activities higher than naturally occurring promoters (see Li et al, Nat. Biotech., 17:241-245 (1999)). Examples of promoters that are tissue-specific are known for liver (albumin, Miyatake et al, J. Virol., 71:5124-32 (1997); hepatitis B virus core promoter, Sandig et al., Gene Ther., 3:1002-9 (1996); alpha-fetoprotein (AFP), Arbuthnot et al, Hum. Gene Ther., 7:1503-14 (1996)), bone osteocalcin (Stein et al, Mol. Biol Rep., 24:185-96 (1997)); bone sialoprotein (Chen et al, J. Bone Miner. Res., 11:654-64 (1996)), lymphocytes (CD2, Hansal et al, J. Immunol, 161: 1063-8 (1998); immunoglobulin heavy chain; T cell receptor chain), neuronal such as neuron-specific enolase (NSE) promoter (Andersen et al, Cell. Mol Neurobiol, 13:503-15 (1993)), neurofilament light-chain gene (Piccioli et al, Proc. Natl Acad. ScL USA, 88:561 1-5 (1991)), and the neuron-specific vgf gene (Piccioli et al, Neuron, 15:373-84 (1995)), among others. Optionally, vectors carrying transgenes encoding therapeutically useful or immunogenic products may also include selectable markers or reporter genes may include sequences encoding geneticin, hygromicin or purimycin resistance, among others. Such selectable reporters or marker genes (preferably located outside the viral genome to be packaged into a viral particle) can be used to signal the presence of the plasmids in bacterial cells, such as ampicillin resistance. Other components of the vector may include an origin of replication. Selection of these and other promoters and vector elements are conventional and many such sequences are available [see, e.g., Sambrook et al, and references cited therein]. These vectors are generated using the techniques and sequences provided herein, in conjunction with techniques known to those of skill in the art. Such techniques include conventional cloning techniques of cDNA such as those described in texts [Sambrook et al, Molecular Cloning: A Laboratory Manual, Cold Spring Harbor Press, Cold Spring Harbor, N.Y.], use of overlapping oligonucleotide sequences of the adenovirus genomes, polymerase chain reaction, and any suitable method which provides the desired nucleotide sequence.

In some embodiments, the adenoviral vector comprising a transgene according to the disclosure comprises, consists, or consists essentially of the nucleic acid sequence of SEQ ID NO: 2. In some embodiments, the adenoviral vector comprises a nucleic acid sequence having at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 2.

In some embodiments, a virus-like particle (VLP) is provided that includes a disclosed recombinant adenoviral vector. VLPs lack the viral components that are required for virus replication and thus represent a highly attenuated, replication-incompetent form of a virus. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al, Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al, J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

### b) Components of the Composition

The present disclosure also provides pharmaceutical compositions. The pharmaceutical composition comprises an adenoviral composition of the present disclosure, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the adenoviral composition of the present disclosure. Thus, in addition to the therapies described herein, one may also provide to the subject other therapies known to be efficacious for treatment of a viral infection. In some embodiments, the secondary agent is selected from a corticosteroid, a non-steroidal anti-inflammatory drug (NSAID), an intravenous immunoglobulin, a kinase inhibitor, a fusion or recombinant protein, a monoclonal antibody, or a combination thereof. In some embodiments, agents suitable for combination therapy include but are not limited to inhaled bronchodilators and inhaled steroids.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

A formulation comprising a composition for intranasal deliver may have a pH corresponding to a physiologically acidic nasal pH. The physiologically acidic nasal pH may depend on intact nasal mucosal function. A composition may comprise a pH of about be 6.5±0.5 (5.9 to 7.3) or about 6.7±0.6 (5.3 to 7.6). A composition may comprise a pH of about 3.8-7.7 (mean±SD 5.7±0.9). A composition for nasal deliver may be in the slightly acidic range. The average pH may have an acidity of pH 5.7.

Effective delivery of therapeutic agents via intranasal administration must take into account the decreased transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant disclosure optionally incorporate effective mucolytic or mucus-clearing agents, which serve to degrade, thin or clear mucus from intranasal mucosal surfaces to facilitate absorption and/or adsorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. a-amylase and B-amylase are representative of this class of enzymes, although their mucolytic effect may be limited. In contrast, bacterial glycosidases which allow these microorganisms to permeate mucus layers of their hosts.

For combinatorial use with the adenoviral composition within the disclosure, non-ionogenic detergents are generally also useful as mucolytic or mucus-clearing agents. These agents typically will not modify or substantially impair the activity of the adenoviral composition.

### c) Administration

(i) Dosage Forms

The composition can be formulated into various dosage forms and administered by a number of different means that will deliver a therapeutically effective amount of the active ingredient. Such compositions can be administered orally (e.g. inhalation), or parenterally in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

For parenteral administration (including subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

Generally, a safe and effective amount of an adenoviral composition is administered, for example, that amount that would cause the desired therapeutic effect in a subject while minimizing undesired side effects. In various embodiments, an effective amount of an adenoviral composition described herein can substantially reduce viral infectivity in a subject suffering from a viral infection. In some embodiments, an effective amount is an amount capable of treating a respiratory viral infection. In some embodiments, an effective amount is an amount capable of treating one or more symptoms associated with a respiratory viral infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Dosages of the adenoviral vector will depend primarily on factors such as the condition being treated, the age, weight and health of the patient, and may thus vary among patients. For example, a therapeutically effective adult human or veterinary dosage of the viral vector is generally in the range of from about 100 pL to about 100 mL of a carrier containing concentrations of from about 1×10⁶ to about 1×10¹⁵ particles, about 1×10⁷ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the transgene, or for an immunogen, the level of circulating antibody, can be monitored to determine the frequency of dosage administration. Yet other methods for determining the timing of frequency of administration will be readily apparent to one of skill in the art.

An optional method step involves the co-administration to the patient, either concurrently with, or before or after administration of the viral vector, of a suitable amount of a short acting immune modulator. The selected immune modulator is defined herein as an agent capable of inhibiting the formation of neutralizing antibodies directed against the recombinant vector of this invention or capable of inhibiting cytolytic T lymphocyte (CTL) elimination of the vector. The immune modulator may interfere with the interactions between the T helper subsets (T_{H}i or T\hat{ }) and B cells to inhibit neutralizing antibody formation. Alternatively, the immune modulator may inhibit the interaction between T_{H}i cells and CTLs to reduce the occurrence of CTL elimination of the vector. A variety of useful immune modulators and dosages for use of same are disclosed, for example, in Yang et al., J. Virol., 70(9) (September, 1996); International Patent Application Publication No. WO 96/12406, published May 2, 1996; and International Patent Application No. PCT/US96/03035, all incorporated herein by reference

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a viral composition can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a respiratory virus.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal.

### II. Methods

The present disclosure encompasses methods to treat, prevent, or reduce the infectivity or transmission of a virus in a subject in need thereof. In some embodiments, the methods prevent or reduce the infectivity of a viral infection by preventing internalization of a virus into a cell of the subject or by preventing internalization of a viral genome into a cell of the subject. In some embodiments, administration of a composition provided herein, for instance those described in Section II, may create an immune response in a subject which disrupts or prevents an interaction between a viral surface protein (e.g., a spike protein or an envelope protein) and a host receptor protein (e.g., an epithelial angiotensin converting enzyme (ACE)). Administering a composition of the disclosure to a subject at risk for a viral infection may reduce the risk of coronavirus infection in the subject.

The disclosed compositions can be administered to a subject to induce an immune response to the corresponding coronavirus spike protein in the subject. In a particular example, the subject is a human. The immune response can be a protective immune response, for example a response that inhibits subsequent infection with the corresponding coronavirus. Elicitation of the immune response can also be used to treat or inhibit infection and illnesses associated with the corresponding coronavirus.

A subject can be selected for treatment that has, or is at risk for developing infection with the coronavirus corresponding to the S protein in the immunogen, for example because of exposure or the possibility of exposure to the coronavirus. Following administration of a disclosed immunogen, the subject can be monitored for infection or symptoms associated with the coronavirus, or both.

Typical subjects intended for treatment with the therapeutics and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional workups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed compositions can be for prophylactic or therapeutic purpose. When provided prophylactically, the disclosed therapeutic agents are provided in advance of any symptom, for example, in advance of infection. The prophylactic administration of the disclosed therapeutic agents serves to prevent or ameliorate any subsequent infection. When provided therapeutically, the disclosed therapeutic agents are provided at or after the onset of a symptom of disease or infection, for example, after development of a symptom of infection with the coronavirus corresponding to the S protein in the composition, or after diagnosis with the coronavirus infection. The therapeutic agents can thus be provided prior to the anticipated exposure to the coronavirus so as to attenuate the anticipated severity, duration or extent of an infection and/or associated disease symptoms, after exposure or suspected exposure to the virus, or after the actual initiation of an infection.

The compositions described herein, are provided to a subject in an amount effective to induce or enhance an immune response against the coronavirus S protein in the subject, preferably a human. The actual dosage of disclosed compositions will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A composition according to the disclosure can be used in coordinate (or prime-boost) vaccination protocols or combinatorial formulations. In certain embodiments, compositions and coordinate immunization protocols employ separate transgene or formulations, each directed toward eliciting an anti-viral immune response, such as an immune response to coronavirus S proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed transgene. In some examples that the boost may be the same transgene as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, a composition of the disclosure is administered in a single-dose.

Upon administration of a disclosed composition of this disclosure, the immune system of the subject typically responds to the composition by producing antibodies specific for the coronavirus S protein included in the composition. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant coronavirus S protein included in the immunogen.

Coronavirus infection does not need to be completely eliminated or reduced or prevented for the methods to be effective. For example, elicitation of an immune response to a coronavirus with one or more of the disclosed compositions can reduce or inhibit infection with the coronavirus by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection with the coronavirus in the absence of the composition. In additional examples, coronavirus replication can be reduced or inhibited by the disclosed methods. Coronavirus replication does not need to be completely eliminated for the method to be effective. For example, the immune response elicited using one or more of the disclosed compositions can reduce replication of the corresponding coronavirus by a desired amount, for example, by at least 10%, at least 20%, at least 50%, at least 60%, at least 70%, at least 80%, at least 90%, at least 95%, at least 98%, or even at least 100% (elimination or prevention of detectable replication of the coronavirus), as compared to replication of the coronavirus in the absence of the immune response.

In some embodiments, the disclosed composition is administered to the subject simultaneously with the administration of the adjuvant. In other embodiments, the disclosed composition is administered to the subject after the administration of the adjuvant and within a sufficient amount of time to induce the immune response.

In some embodiments, administration of a therapeutically effective amount of one or more of the disclosed compositions to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays. In some embodiments, the serum neutralization activity can be assayed using a panel of coronavirus pseudoviruses. For example, to test the immunogenicity of the vaccine candidates against multiple MERS-CoV strains—without the requirement of a biosafety level 3 facility—a pseudotyped reporter virus neutralization assay was previously developed (Wand et al., Nat Commun, 6:7712, 2015), similar to that previously developed for SARS-CoV (Martin et al, Vaccine 26, 6338, 2008; Yang et al, Nature 428, 561, 2004; Naldini et al, PNAS 93, 11382, 1996; Yang et al, PNAS 102, 797, 2005).

In other embodiments, the present disclosure provides methods to treat, prevent, or reduce the infectivity of a respiratory viral infection. In some embodiments, the viral infection may be a coronavirus infection. The coronavirus may be SARS-CoV, SARS-CoV-2, MERS-CoV, HKU1, OC43, or 229E. The coronavirus may be a beta-coronavirus. A subject at risk for a coronavirus infection may come in contact with an asymptomatic carrier of the coronavirus infection, thereby unknowingly contracting the coronavirus infection.

Generally, the methods as described herein comprise administration of a therapeutically effective amount of a nanoparticle composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a nanoparticle composition, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: A Single-Dose Chimpanzee Adenovirus Vectored Vaccine Protects Against SARS-COV-2 Infection and Pneumonia in Mice Expressing Human ACE2 Receptor

The present example provides a chimpanzee Ad (simian AdV-36)-based SARS-CoV-2 vaccine (ChAd-SARS-CoV-2-S) encoding a pre-fusion stabilized spike (S) protein after introducing two proline substitutions in the S2 subunit. Intramuscular administration of ChAd-SARS-CoV-2-S induced robust systemic humoral and cell-mediated immune responses against the S protein. One or two vaccine doses protected against lung infection, inflammation, and pathology after SARS-CoV-2 challenge of mice that transiently express the human ACE2 (hACE2) receptor. Despite the induction of high levels of neutralizing antibody in serum, neither dosing regimen completely protected against SARS-CoV-2 infection, as substantial levels of viral RNA were still detected in the lung. In comparison, when a single dose of ChAd-SARS-CoV-2-S by the intranasal route was administered, high levels of neutralizing antibody and anti-SARS-CoV-2 IgA, and complete protection against infection in both the upper and lower respiratory tracts were detected. Moreover, and in contrast to the control ChAd vaccine, 8 days after SARS-CoV-2 challenge, serum antibody responses to the SARS-CoV-2 NP protein were absent in animals immunized with ChAd-SARS-CoV-2-S via intranasal delivery. Thus, ChAd-SARS-CoV-2-S has the potential to confer sterilizing immunity at the site of inoculation, which prevent both virus-induced disease and transmission.

Results

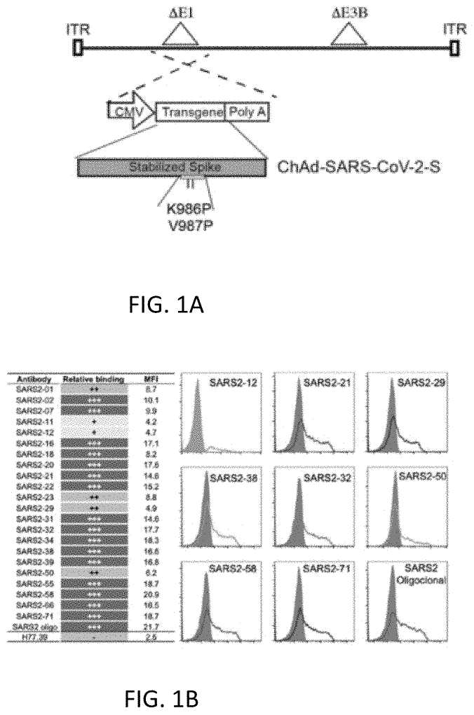

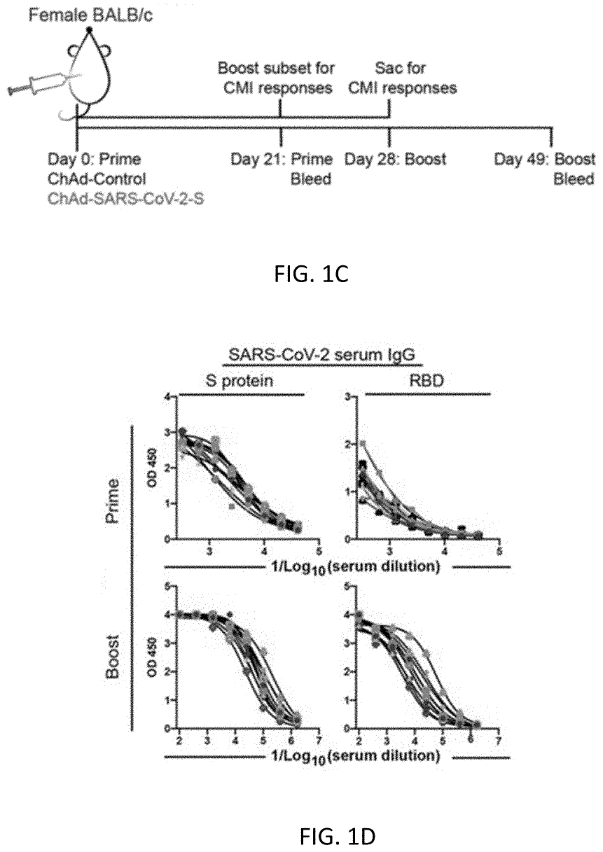

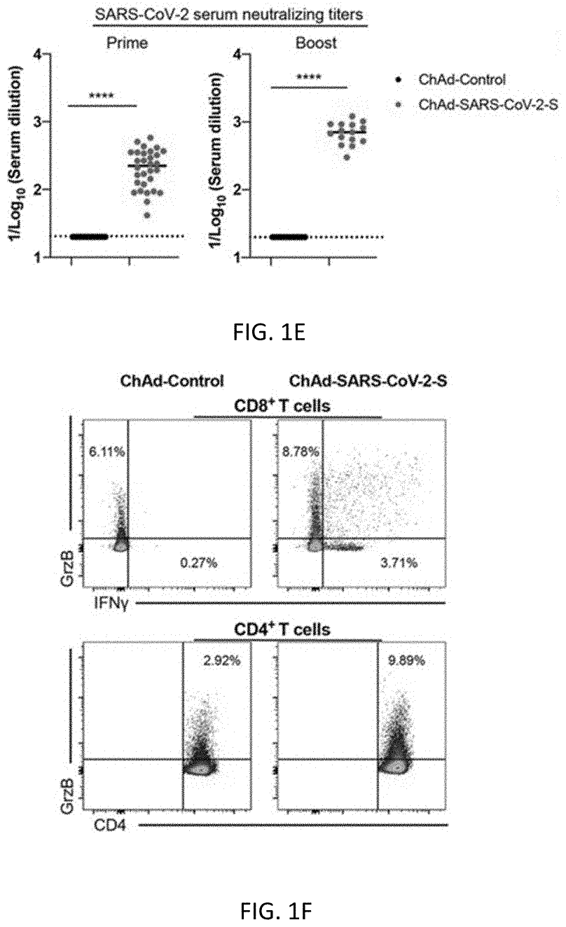

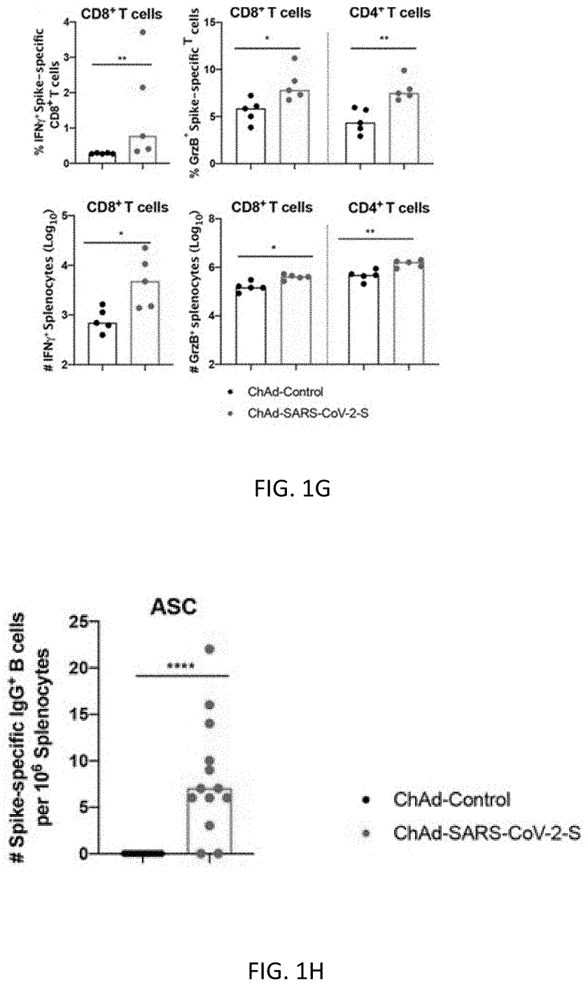

Chimpanzee Ad-vectored vaccine induces robust antibody responses against anti-SARS-CoV-2: Two replication-incompetent ChAd vectors based on a simian Ad-36 virus were constructed. The ChAd-SARS-CoV-2-S vector encodes the full-length sequence of SARS-CoV-2 S protein as a transgene including the ectodomain, transmembrane domain, and cytoplasmic domain (GenBank: QJQ84760.1) and is stabilized in pre-fusion form by two proline substitutions at residues K986 and V987. The ChAd-control has no transgene. The S protein transgene is controlled transcriptionally by a cytomegalovirus promoter. To make the vector replication-incompetent and enhance packaging capacity, the E1A/B genes were replaced and a deletion in the E3B gene introduced, respectively (FIG. 1A). To confirm that the S protein was expressed and antigenically intact, 293T cells were transduced and binding of a panel of 22 neutralizing monoclonal antibodies against the S protein confirmed by flow cytometry (FIG. 1B).

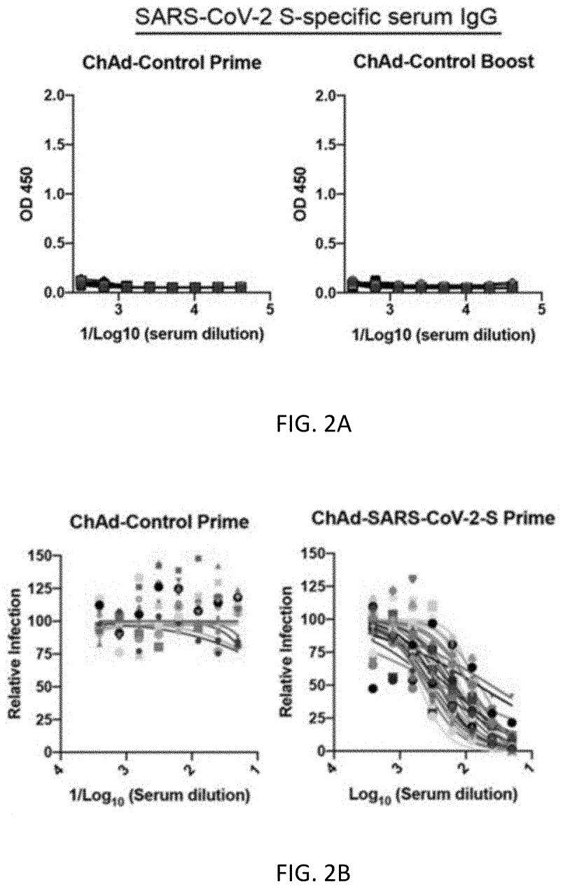

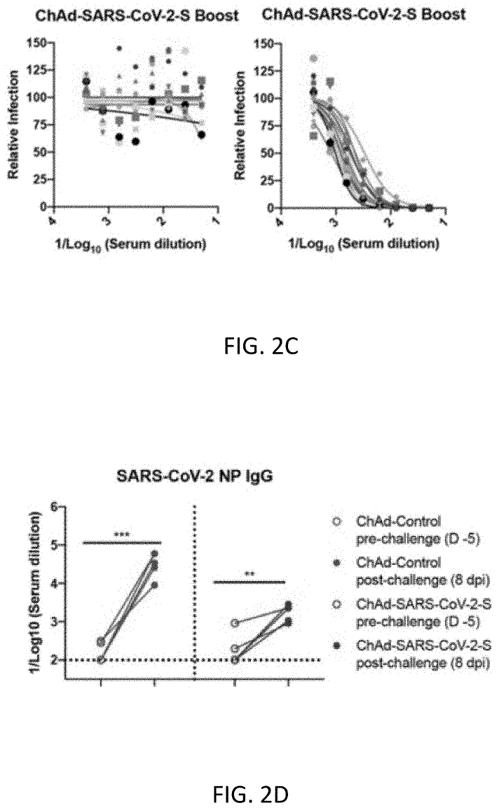

To assess the immunogenicity of ChAd-SARS-CoV-2-S, groups of 4-week-old BALB/c mice were immunized by intramuscular inoculation with 1010 virus particles of ChAd-SARS-CoV-2-S or ChAd-control. Some mice received a booster dose four weeks later. Serum samples were collected 21 days after primary or booster immunization (FIG. 1C), and IgG responses against purified S and RBD proteins were evaluated by ELISA. Whereas ChAd-SARS-CoV-2-S induced high levels of S- and RBD-specific IgG, low, if any levels were detected in the ChAd-control-immunized mice (FIG. 1D and FIG. 2A). Serum samples were assayed in vitro for neutralization of infectious SARS-CoV-2 using a focus-reduction neutralization test (FRNT). As expected, serum from ChAd-control-immunized mice did not inhibit SARS-CoV-2 infection after primary immunization or boosting. In contrast, serum from ChAd-SARS-CoV-2-S vaccinated animals strongly neutralized SARS-CoV-2 infection, and boosting enhanced this inhibitory activity (FIG. 1E and FIG. 2B-2C).

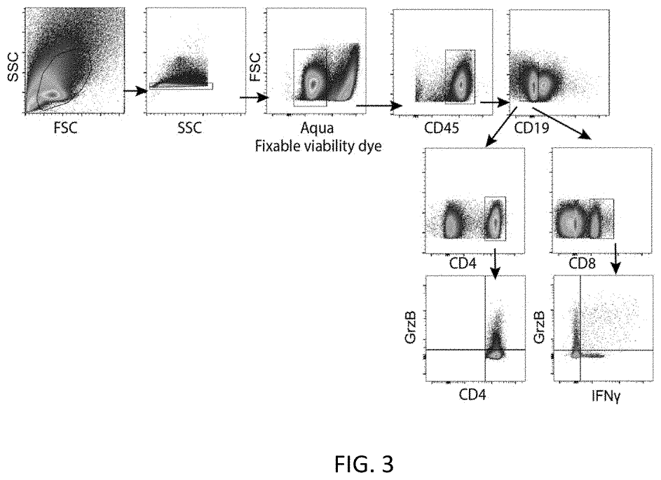

Vaccine-induced memory CD8+ T cell and antigen specific B cell responses: Because optimal vaccine immunity often is comprised of both humoral and cellular responses, the levels of SARS-CoV-2-specific CD4+ and CD8+ T cells after vaccination were measured. Four-week old BALB/c mice were immunized with ChAd-SARS-CoV-2-S or ChAd-control and boosted three weeks later. To assess the vaccine-induced SARS-CoV-2-specific CD4+ and CD8+ T cell responses, splenocytes were harvested one week after boosting and stimulated ex vivo with a pool of 253 overlapping 15-mer S peptides. Subsequently, quantification of intracellular IFNγ and granzyme B expression was determined by flow cytometry. After peptide re-stimulation ex vivo, splenic CD8+ T cells expressed IFNγ and both splenic CD4+ and CD8+ T cells expressed granzyme B in mice immunized with ChAd-SARS-CoV-2-S but not the ChAd-control vector (FIGS. 1F-1G and FIG. 3). To assess the antigen-specific B cell responses, splenocytes were harvested and subjected to an ELISPOT analysis with S protein. The ChAd-SARS-CoV-2-S vaccine induced S protein-specific IgG antibody-secreting cells in the spleen whereas the control vaccine did not (FIG. 1H).

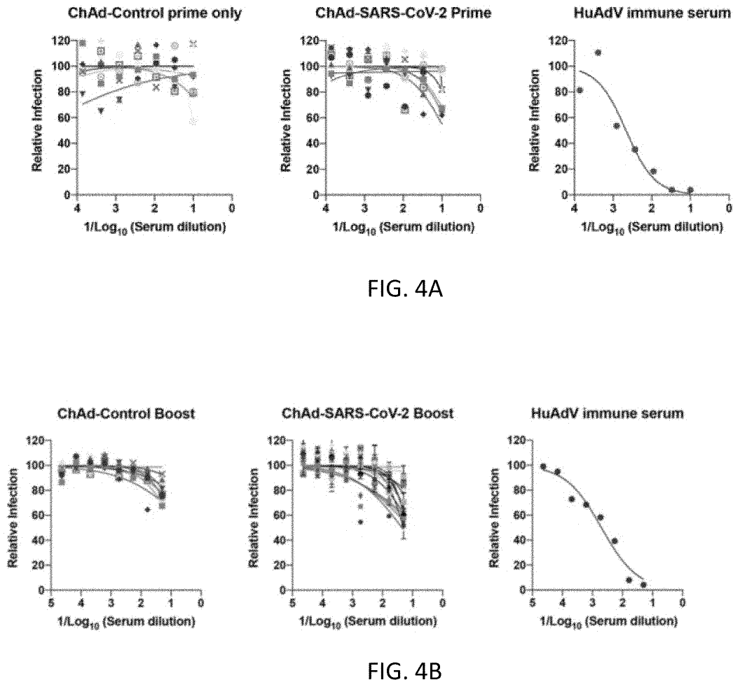

Intramuscular immunization with ChAd-SARS-CoV-2-S vaccine protects against SARS-CoV-2 infection in the lung: The protective activity of the ChAd vaccine in a recently developed SARS-CoV-2 infection model wherein BALB/c mice express hACE2 in the lung after intranasal delivery of a vectored human Ad (Hu-Ad5-hACE2) was tested. Endogenous mouse ACE2 does not support viral entry, and this system enables productive SARS-CoV-2 infection in the mouse lung. Four-week-old BALB/c mice first were immunized via an intramuscular route with ChAd-control or ChAd-SARS-CoV-2-S vaccines. Approximately thirty days later, mice were administered 10⁸ plaque-forming units (PFU) of Hu-Ad5-hACE2 and anti-Ifnar1 monoclonal antibody (mAb) via intranasal and intraperitoneal routes, respectively. A single dose of anti-Ifnar1 mAb was also administered to enhance lung pathogenesis in this model. The absence of cross-immunity between the ChAd and the Hu-Ad5 vector was confirmed. Serum from ChAd-immunized mice did not neutralize Hu-Ad5 infection (FIG. 4A-4B).

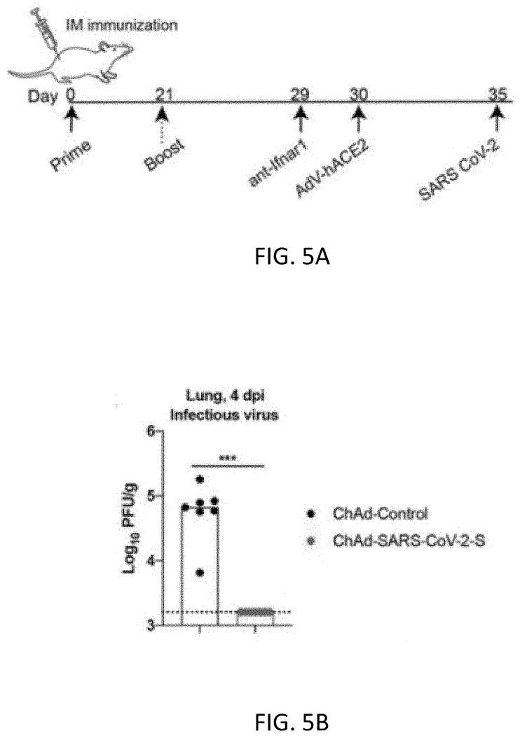

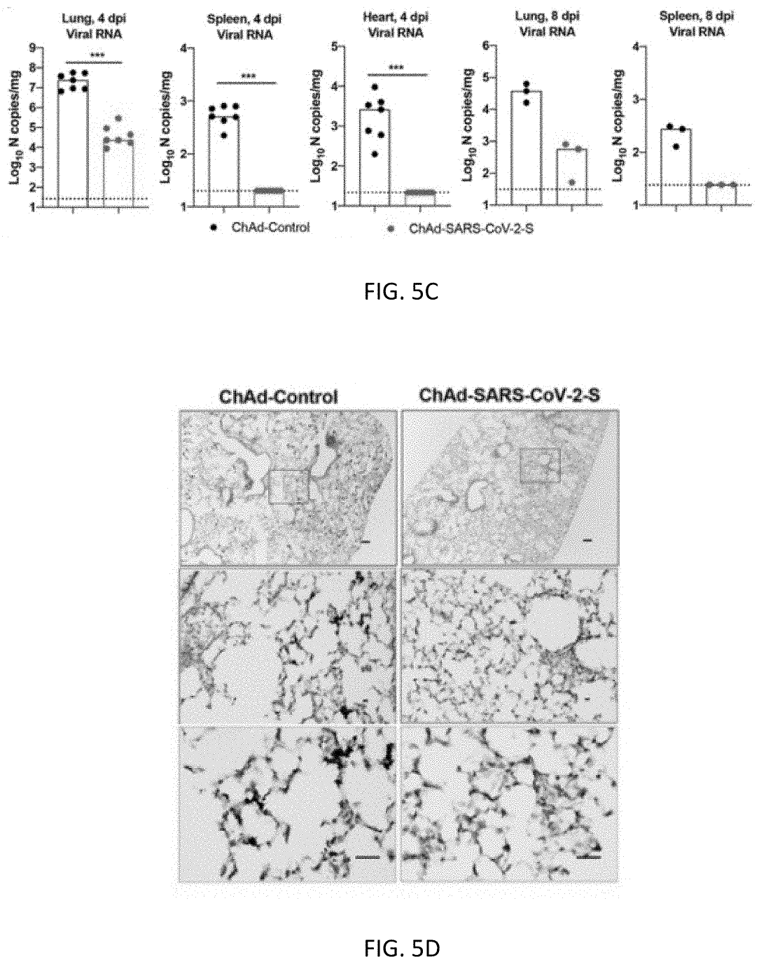

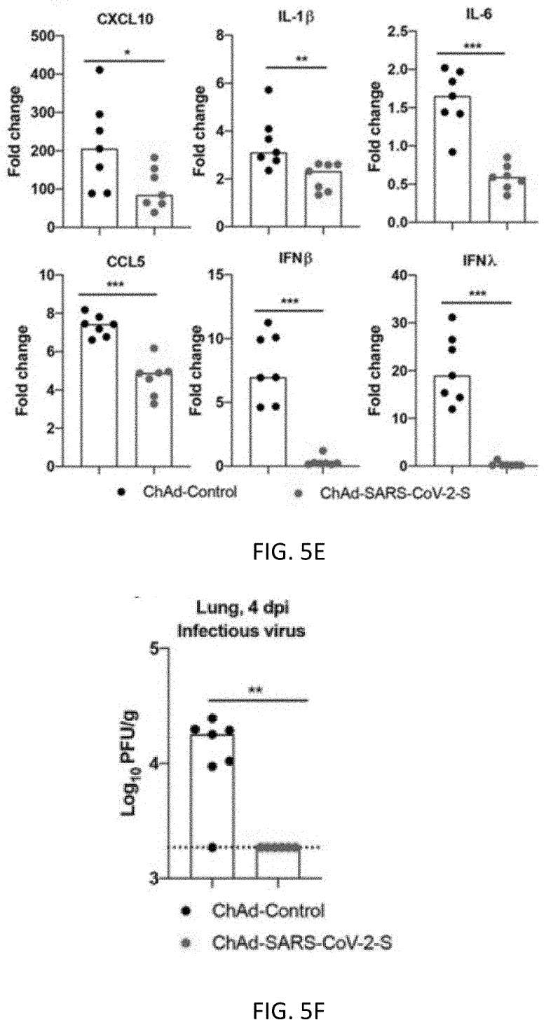

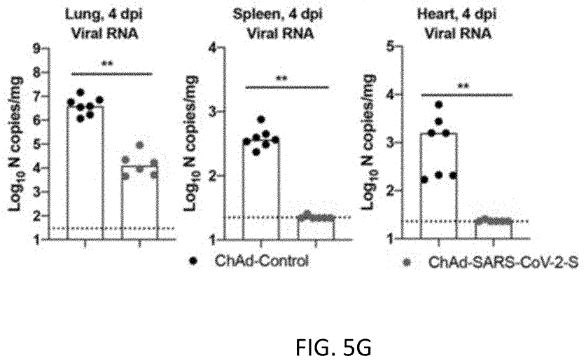

Five days after Hu-Ad5-hACE2 transduction, mice were challenged via intranasal route with 4×10⁵ focus-forming units (FFU) of SARS-CoV-2 (FIG. 2A). At 4 days post-infection (dpi), the peak of viral burden in this model, mice were euthanized and lungs, spleen, and heart were harvested for viral burden and cytokine analysis. Notably, there was no detectable infectious virus in the lungs of mice immunized with ChAd-SARS-CoV-2-S as determined by plaque assay, whereas high levels were present in mice vaccinated with ChAd-control (FIG. 5B). Consistent with this result, reduced viral RNA levels in the lung, heart, and spleen of ChAd-SARS-CoV-2-S vaccinated animals were observed compared to mice receiving the ChAd-control vector (FIG. 5C). In situ hybridization staining for viral RNA in lungs harvested at 4 dpi revealed a substantial decrease of SARS-CoV-2 RNA in pneumocytes of animals immunized with ChAd-SARS-CoV-2-S compared to the ChAd-control (FIG. 5D). A subset of immunized animals was euthanized at 8 dpi, and tissues were harvested for evaluation. At this time point, viral RNA levels again were lower or absent in the lung and spleen of ChAd-SARS-CoV-2-S immunized mice compared to the control ChAd vector (FIG. 5C). Collectively, these data indicate that a single intramuscular immunization with ChAd-SARS-CoV-2-S results in markedly reduced, but not abrogated, SARS-CoV-2 infection in the lungs of challenged mice.

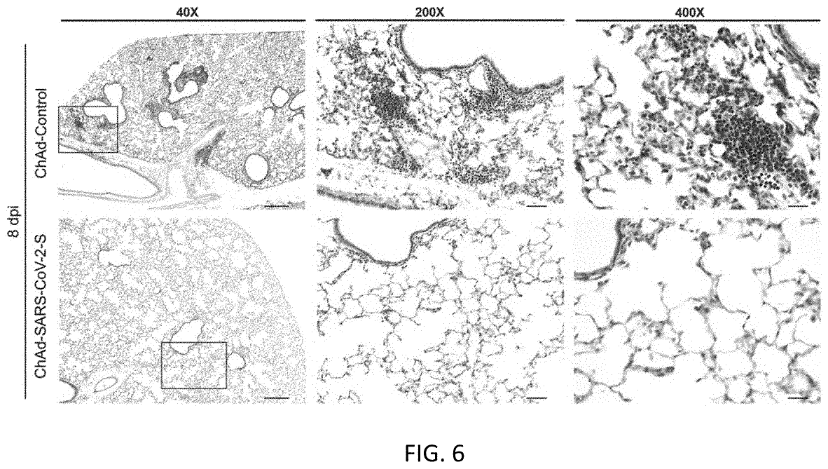

Next, the effect of the vaccine on lung inflammation and disease were assessed. Several proinflammatory cytokines and chemokine mRNA levels were lower in the lung tissues of animals immunized with ChAd-SARS-CoV-2-S compared to ChAd-control including CXCL10, IL1β, IL-6, CCL5, IFNβ and IFNγ (FIG. 5E). Moreover, mice immunized with ChAd-control vaccine and challenged with SARS-CoV-2 showed evidence of viral pneumonia characterized by immune cell accumulation in perivascular and alveolar locations, vascular congestion, and interstitial edema. In contrast, animals immunized with ChAd-SARS-CoV-2-S showed a marked attenuation of the inflammatory response in the lung that develops in the ChAd-control-immunized mice (FIG. 6). Thus, immunization with Ch-Ad-SARS-CoV-2 decreases both viral infection and the consequent lung inflammation and injury associated with SARS-CoV-2 infection.

Improved protection using a prime-boost vaccine regimen was then assessed. BALB/c mice were immunized via an intramuscular route with ChAd-control or ChAd-SARS-CoV-2-S and received a homologous booster dose four weeks later. At day 29 post-boost, mice were treated with a single dose of anti-Ifnar1 antibody followed by Hu-Ad5-hACE2 and then challenged with SARS-CoV-2 five days later. As expected, the prime-boost regimen protected against SARS-CoV-2 challenge with no infectious virus detected in the lungs (FIG. 5F). Although marked reductions of viral RNA in the lung, spleen, and heart were detected at 4 dpi, residual levels of viral RNA still were present suggesting protection was not complete, even after boosting (FIG. 5G).

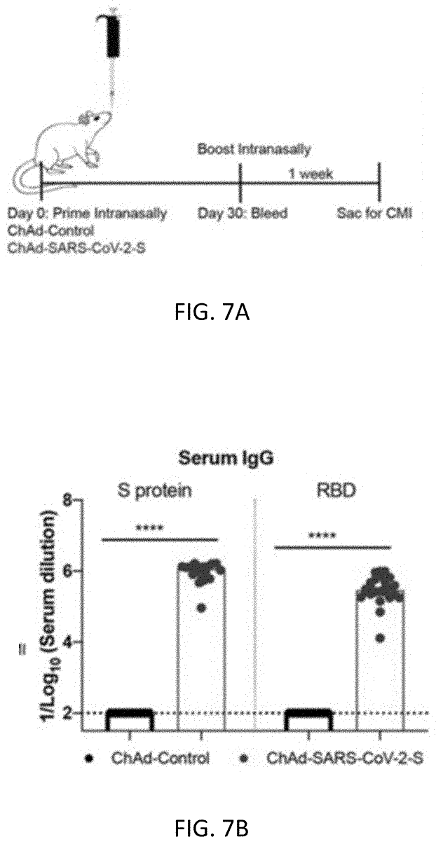

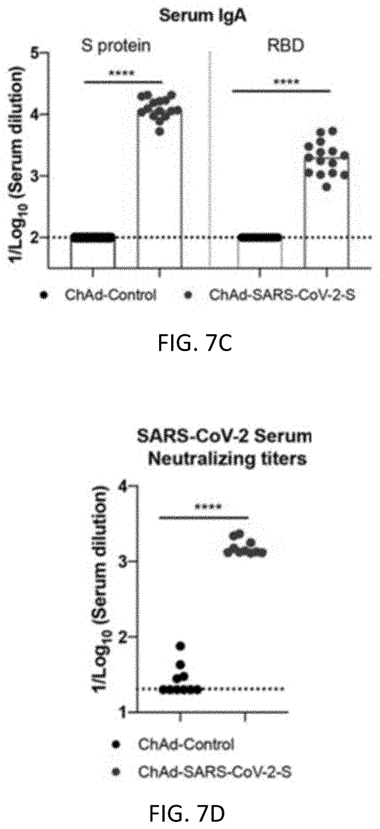

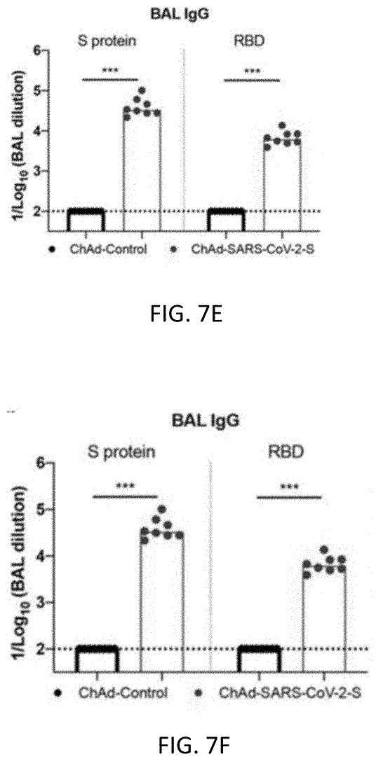

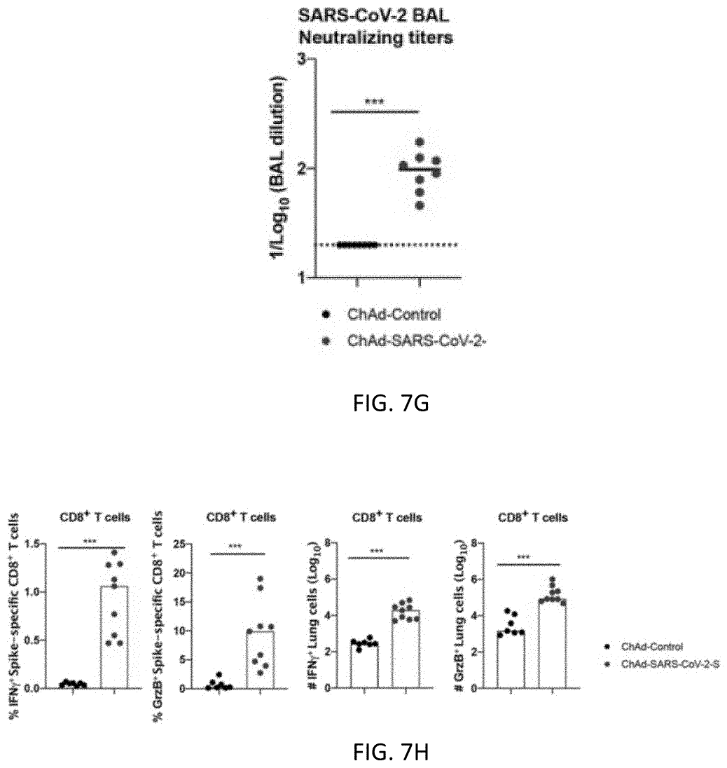

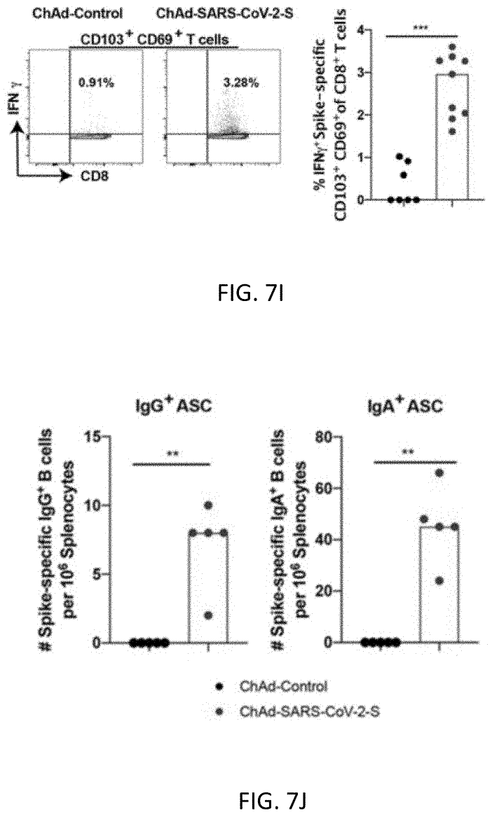

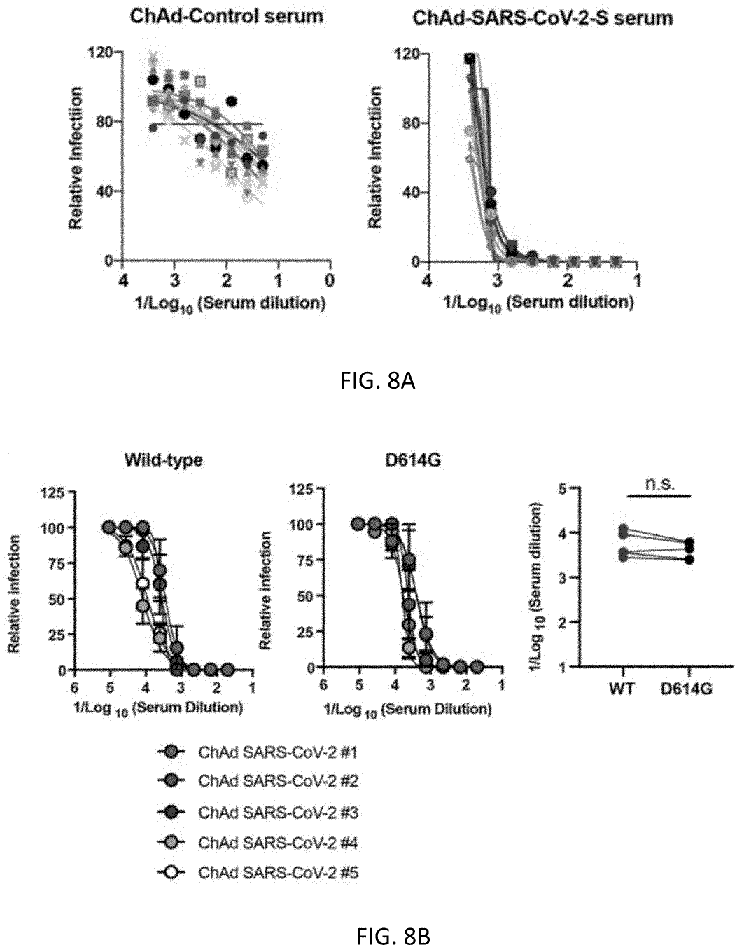

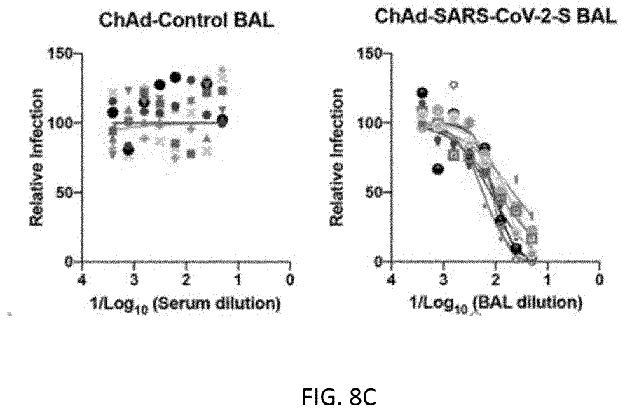

A single intranasal immunization with ChAd-SARS-CoV-2-S induces sterilizing immunity against SARS-CoV-2: Mucosal immunization through the nasopharyngeal route can elicit local immune responses including secretory IgA antibodies that confer protection at or near the site of inoculation of respiratory pathogens. To assess the immunogenicity and protective efficacy of mucosal vaccination, five-week old BALB/c mice were inoculated via intranasal route with 10¹⁰ viral particles of ChAd-control or ChAd-SARS-CoV-2-S (FIG. 7A). Serum samples were collected at four weeks post-immunization to evaluate humoral immune responses. Intranasal immunization of ChAd-SARS-CoV-2-S but not ChAd-control induced high levels of S- and RBD-specific IgG and IgA (FIGS. 7B-7C) and SARS-CoV-2 neutralizing antibodies (geometric mean titer of 1/1,574) in serum (FIG. 7D and FIG. 8A). Serum antibodies from mice immunized with ChAd-SARS-CoV-2-S equivalently neutralized a recombinant, luciferase-expressing variant of SARS-CoV-2 encoding a D614G mutation in the S protein (FIG. 8B); this finding is important, because many circulating viruses contain this substitution, which is associated with greater infectivity in cell culture (doi.org/10.1101/2020.06.12.148726). The SARS-CoV-2-specific antibody responses in bronchoalveolar lavage (BAL) fluid of immunized mice was also assessed. BAL fluid from ChAd-SARS-CoV-2-S but not ChAd-control vaccinated mice showed high levels of S- and RBD-specific IgG and IgA antibodies (FIG. 7E-7F) including those with neutralizing activity (FIG. 7G and FIG. 8C).

To assess T cell responses activated via mucosal immunization, mice were vaccinated via intranasal route with either ChAd-SARS-CoV-2-S or ChAd-control and boosted similarly four weeks later. Lungs were harvested one-week post-boosting, and T cells were analyzed by flow cytometry. Re-stimulation ex vivo with a pool of S peptides resulted in a marked increase in IFNγ and granzyme B producing CD8+ T cells in the lungs of mice that received the ChAd-SARS-CoV-2-S vaccine (FIG. 7H). Specifically, a population of IFNγ-secreting, antigen-specific CD103⁺CD69⁺CD8⁺ T cells in the lung was identified (FIG. 7I) which is phenotypically consistent with vaccine-induced resident memory T cells. In the spleen, antibody-secreting plasma cells producing IgA or IgG against the S protein were detected after intranasal immunization with ChAd-SARS-CoV-2-S (FIG. 7J). Of note, an ˜5-fold higher frequency of B cells secreting anti-S IgA than IgG was observed.

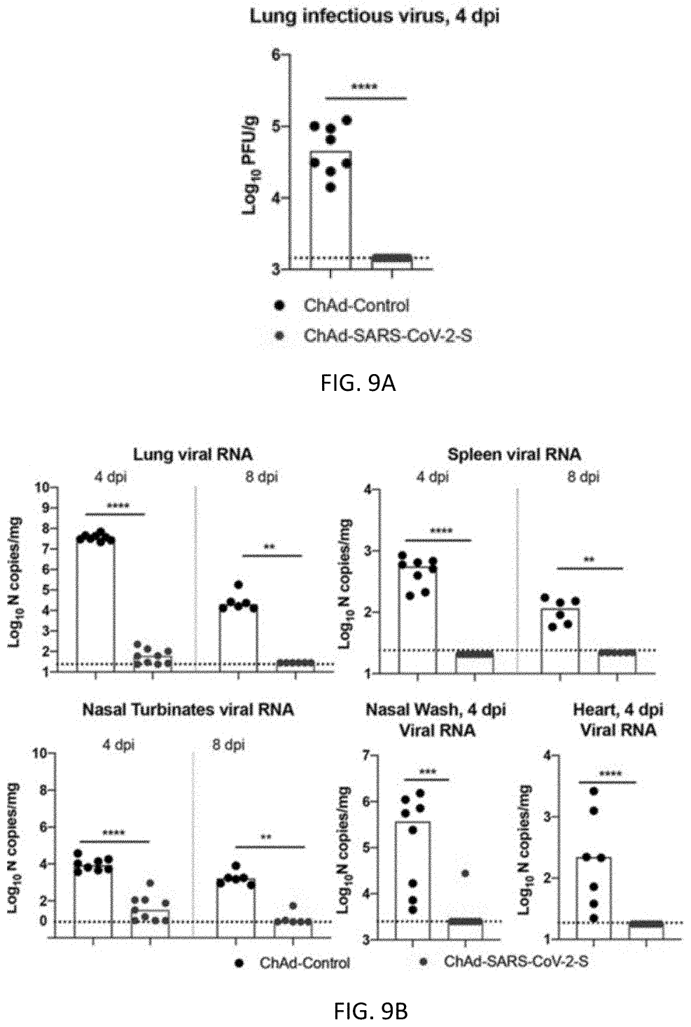

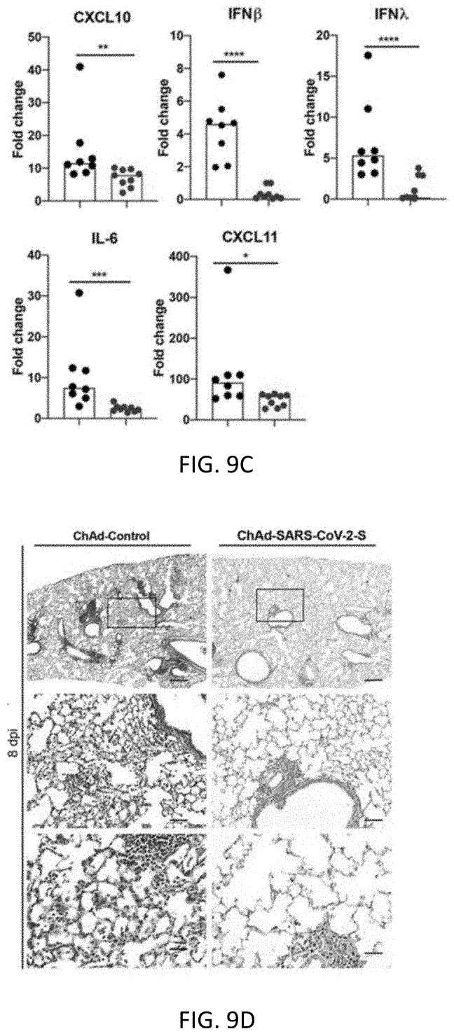

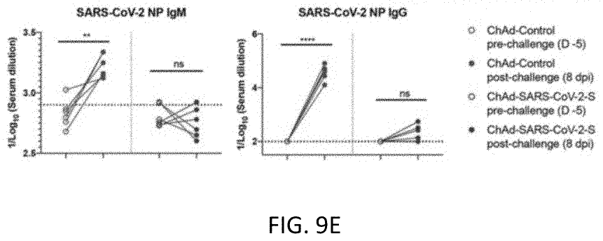

The protective efficacy of the ChAd vaccine after single-dose intranasal immunization was evaluated. At day 30 post-vaccination, mice were administered 10⁸ PFU of Hu-Ad5-hACE2 and anti-Ifnar1 mAb as described above. Five days later, mice were challenged with 4×10⁵ FFU of SARS-CoV-2. At 4 and 8 dpi, lungs, spleen, heart, nasal turbinates, and nasal washes were harvested and assessed for viral burden. Intranasal delivery of the ChAd-SARS-CoV-2-S vaccine demonstrated remarkable protective efficacy as judged by an absence of infectious virus in the lungs (FIG. 9A) and almost no measurable viral RNA in the lung, spleen, heart, nasal turbinates, or nasal washes (FIG. 9B). The very low viral RNA levels in the lung and nasal turbinates at 4 dpi likely reflects the input, non-replicating virus, as similar levels were measured at this time point in C57BL/6 mice lacking hACE2 receptor expression. Cytokine and chemokine mRNA levels in lung homogenates also were substantially lower in mice immunized with the ChAd-SARS-CoV-2-S than the ChAd-control vaccine (FIG. 9C), with residual expression likely due to the human Ad vector hACE2 delivery system. Finally, histopathological analysis of lung tissues from animals vaccinated with ChAd-SARS-CoV-2-S by intranasal route and challenged with SARS-CoV-2 showed minimal, if any, perivascular and alveolar infiltrates at 8 dpi compared to the extensive inflammation observed in ChAd-control vaccinated animals (FIG. 9D).

To determine if sterilizing immunity was achieved with intranasal delivery of ChAd-SARS-CoV-2-S, anti-NP antibodies were measured at 8 dpi and compared to responses from 5 days before SARS-CoV-2 infection. Because the NP gene is absent from the vaccine vector, induction of humoral immune responses against NP after SARS-CoV-2 exposure suggests viral protein translation and active infection. After SARS-CoV-2 challenge, anti-NP antibody responses were detected in ChAd-control mice vaccinated by an intranasal route or ChAd-control and ChAd-SARS-CoV-2-S mice vaccinated by an intramuscular route (FIG. 9E and FIG. 2D). Remarkably, none of the mice immunized with ChAd-SARS-CoV-2-S via an intranasal route showed significant increases in anti-NP antibody responses after SARS-CoV-2 infection. Combined with our virological analyses, these data suggest that a single intranasal immunization of ChAd-SARS-CoV-2-S induces robust and likely sterilizing mucosal immunity, which prevents SARS-CoV-2 infection in the upper and lower respiratory tracts of mice expressing the hACE2 receptor.

Discussion

In this example, intramuscular and intranasal administration of a replication-incompetent ChAd vector as a vaccine platforms for SARS-CoV-2 were evaluated. Single dose immunization of a stabilized S protein-based vaccine via an intramuscular route induced S- and RBD-specific binding as well as neutralizing antibodies. Vaccination with one or two doses protected mice expressing human ACE2 against SARS-CoV-2 challenge, as evidenced by an absence of infectious virus in the lungs and substantially reduced viral RNA levels in lungs and other organs. Mice immunized with ChAd-SARS-CoV-2-S also showed markedly reduced if not absent lung pathology, lung inflammation, and evidence of pneumonia compared to the control ChAd vaccine. However, intramuscular vaccination of ChAd-SARS-CoV-2-S did not confer sterilizing immunity, as evidenced by detectable viral RNA levels in several tissues including the lung and induction of anti-NP antibody responses. Mice immunized with a single dose of the ChAd-SARS-CoV-2-S via an intranasal route also were protected against SARS-CoV-2 challenge. Intranasal vaccination, however, generated robust IgA and neutralizing antibody responses that protected against both upper and lower respiratory tract SARS-CoV-2 infection and inhibited infection of both wild-type and D614G variant viruses. The very low viral RNA in upper airway tissues and absence of serological response to NP in the context of challenge strongly suggests that most animals receiving a single intranasal dose of ChAd-SARS-CoV-2-S achieve sterilizing immunity.

Although several vaccine candidates (e.g., lipid-encapsulated mRNA, DNA, inactivated, and viral-vectored) rapidly advanced to human clinical trials in an expedited effort to control the pandemic, few studies have demonstrated efficacy in pre-clinical models. Rhesus macaques immunized with two or three doses of a DNA plasmid vaccine encoding full-length SARS-CoV-2 S protein induced neutralizing antibody in sera and reduced viral burden in BAL and nasal mucosa fluids. Moreover, three immunizations over two weeks with purified, inactivated SARS-CoV-2 induced neutralizing antibodies, and depending on the dose administered provided partial or complete protection against infection and viral pneumonia in rhesus macaques. One limitation of these challenge models is that rhesus macaques develop mild interstitial pneumonia after SARS-CoV-2 infection compared to some humans and other non-human primate species. The present example, in hACE2-expressing mice, showed that a single intramuscular or intranasal dose of ChAd-SARS-CoV-2-S vaccine confers substantial and possibly complete protection against viral replication, inflammation, and lung disease.

The present disclosure supports the use of non-human Ad-vectored vaccines against emerging RNA viruses including SARS-CoV-2. Previous work showed the efficacy of single-dose or two-dose regimens of a gorilla Ad encoding the prM-E genes of Zika virus (ZIKV) in several mouse challenge models including in the context of pregnancy. Others have evaluated ChAd or rhesus macaque Ad vaccine candidates against ZIKV and shown efficacy in mice and non-human primates. A different ChAd encoding the wild-type SARS-CoV-2 S protein (ChAdOx1) is currently in clinical trials in humans (NCT04324606). Although data from the human trials has not yet been reported, studies in rhesus macaques suggest that a single intramuscular dose protects against infection in the lung but not in the upper respiratory tract (biorxiv.org/content/10.1101/2020.05.13.093195v1). None of the vaccines evaluated against SARS-CoV-2 has shown evidence of immune enhancement in any pre-clinical or clinical study, which has been a theoretical risk based on studies with other human and animal coronaviruses. Indeed, and in contrast to data with SARS-CoV vaccines or antibodies, enhanced infection, immunopathology, or disease in animals immunized with ChAd encoding SARS-CoV-2 S proteins or administered passively transferred monoclonal antibodies was not observed.

ChAd-SARS-CoV-2-S induced SARS-CoV-2 specific CD8⁺ T cell responses including a high percentage and number of IFNγ and granzyme expressing cells after ex vivo S protein peptide restimulation. The induction of robust CD8⁺ T cell responses by the ChAd-SARS-CoV-2-S vaccine is consistent with reports with other simian Ad vectors. ChAd vaccine vectors not only overcome issues of pre-existing immunity against human adenoviruses but also have immunological advantages because they do not induce exhausted T cell responses.

A single intranasal dose of ChAd-SARS-CoV-2-S conferred superior immunity against SARS-CoV-2 challenge, more so than one or two intramuscular immunizations of the same vaccine and dose. Given that the serum neutralizing antibody responses was comparable, it is hypothesized that the greater protection observed after intranasal delivery was because of the mucosal immune responses generated. Indeed, high levels of anti-SARS-CoV-2 IgA were detected in serum and lung, and B cells secreting IgA were detected in the spleen. Moreover, intranasal vaccination also induced SARS-CoV-2-specific CD8+ T cells in the lung including CD103⁺CD69⁺ cells, which are likely of a resident memory phenotype. To our knowledge, none of the SARS-CoV-2 vaccine platforms currently in clinical trials use an intranasal delivery approach. There has been great interest in using intranasal delivery for influenza A virus vaccines because of their ability to elicit local humoral and cellular immune responses. Indeed, sterilizing immunity to influenza A virus re-infection requires local adaptive immune responses in the lung, which is optimally induced by intranasal and not intramuscular inoculation. While there are concerns with administering live-attenuated viral vaccines via an intranasal route, subunit-based or replication-incompetent vectored vaccines are promising for generating mucosal immunity in a safer manner, especially given advances in formulation.

In summary, the present example established that immunization with ChAd-SARS-CoV-2-S induces both neutralizing antibody and antigen-specific CD8+ T cell responses. While a single intramuscular immunization of ChAd-SARS-CoV-2-S confers protection against SARS-CoV-2 infection and inflammation in the lungs, intranasal delivery of ChAd-SARS-CoV-2-S induces mucosal immunity, provides superior protection, and likely promotes sterilizing immunity, at least in mice transiently expressing the hACE2 receptor. Thus, the present example supports intranasal delivery of ChAd-SARS-CoV-2-S as a platform for controlling SARS-CoV-2 infection, disease, and transmission.

Methods

Viruses and cells: Vero E6 (CRL-1586, American Type Culture Collection (ATCC), Vero CCL81 (ATCC), and HEK293 cells were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, and 100 U/ml of penicillin-streptomycin.

SARS-CoV-2 strain 2019 n-CoV/USA_WA1/2020 was obtained from the Centers for Disease Control and Prevention (a gift from Natalie Thornburg). The virus was passaged once in Vero CCL81 cells and titrated by focus-forming assay (FFA) on Vero E6 cells. The recombinant luciferase-expressing full-length SARS-CoV-2 reporter virus (2019 n-CoV/USA_WA1/2020 strain) has been reported previously (Zost et al., 2020), and the D614G variant will be described elsewhere. All work with infectious SARS-CoV-2 was performed in Institutional Biosafety Committee approved BSL3 and A-BSL3 facilities using appropriate positive pressure air respirators and protective equipment.

Mouse experiments: Animal studies were carried out in accordance with the recommendations in the Guide for the Care and Use of Laboratory Animals of the National Institutes of Health. The protocols were approved by the Institutional Animal Care and Use Committee at the Washington University School of Medicine (Assurance number A3381-01). Virus inoculations were performed under anesthesia that was induced and maintained with ketamine hydrochloride and xylazine, and all efforts were made to minimize animal suffering.

Female BALB/c mice were purchased from The Jackson Laboratory (catalog 000651). Four to five-week-old animals were immunized with 1010 viral particles (vp) of ChAdV-empty or ChAd-SARS-CoV-2-S in 50 μl PBS via intramuscular injection in the hind leg or via intranasal inoculation. Subsets of immunized animals were boosted four weeks after primary immunization using the same route used for primary immunization. Vaccinated mice (10 to 11-week-old) were given a single intraperitoneal injection of 2 mg of anti-Ifnar1 mAb (MAR1-5A3, Leinco) one day before intranasal administration of 10⁸ PFU of Hu-AdV5-hACE2. Five days after Hu-AdV5 transduction, mice were inoculated with 4×10⁵ FFU of SARS-CoV-2 by the intranasal route. Animals were euthanized at 4 or 8 dpi, and tissues were harvested for virological, immunological, and pathological analyses.

Construction of chimpanzee adenovirus vectors: Simian Ad36 vector (ChAd) was obtained from the Penn Vector Core of the University of Pennsylvania. The ChAd genome was engineered with deletions in the E1 and E3B region (GenBank: FJ025917.1; nucleotides 455-3026 and 30072-31869, respectively). A modified human cytomegalovirus major immediate early promoter sequence was incorporated in place of the E1 gene in counterclockwise orientation on the complementary DNA strand. CMV modification included an addition of two copies of the tet operator 2 (TetO2) sequence inserted in tandem (5′-TCT CTA TCA CTG ATA GGG AGA TCT CTA TCA CTG ATA GG GA-3′)(SEQ ID NO: 7) between the TATA box and the mRNA start (GenBank: MN920393, nucleotides 174211-174212). SARS-CoV-2 S (encoding a prefusion stabilized mutant with two proline substitutions at residues K986 and V987 that stabilize the prefusion form of S) was cloned into a unique PmeI site under the CMV-tetO2 promoter control in pSAd36 genomic plasmid to generate pSAd36-S. In parallel, a pSAd36-control carrying an empty CMV-tetO2 cassette with no transgene also was generated. The pSAd36-S and pSAd-control plasmids were linearized with Pac restriction enzyme to liberate viral genomes for transfection into T-Rex 293-HEK cells (Invitrogen). The rescued replication-incompetent ChAd-SARS-CoV-2-S and ChAd-Control vectors were scaled up in 293 cells and purified by CsCl density-gradient ultracentrifugation. Viral particle concentration in each vector preparation was determined by spectrophotometry at 260 nm.

Construction of a human adenovirus vector expressing human ACE2: Codon-optimized hACE2 sequences were cloned into the shuttle vector (pShuttle-CMV, Addgene 240007) to generate pShuttle-hACE2. pShuttle-hACE2 was linearized with PmeI and subsequently cotransformed with the HuAdv5 backbone plasmid (pAdEasy-1 vector; Addgene 240005) into E. coli strain BJ5183 to generate pAdV5-ACE2 by homologous recombination. The pAdEasy-1 plasmid containing the HuAdV5 genome has deletions in E1 and E3 genes. hACE2 is under transcriptional control of a cytomegalovirus promotor and is flanked at its 3′ end by a SV40 polyadenylation signal. The pAd-hACE2 was linearized with Pac restriction enzyme before transfection into T-Rex 293 HEK cells (Invitrogen) to generate HuAdv5-hACE2. Recombinant HuAdv5-hACE2 was produced in 293-HEK cells and purified by CsCl density-gradient ultracentrifugation. The viral titer was determined by plaque assay in 293-HEK cells.

In situ RNA hybridization and histology: RNA in situ hybridization was performed using RNAscope 2.5 HD (Brown) (Advanced Cell Diagnostics) according to the manufacturer's instructions. Left lung tissues were collected at necropsy, inflated with 10% neutral buffered formalin (NBF), and thereafter immersion fixed in 10% NBF for seven days before processing. Paraffin-embedded lung sections were deparaffinized by incubating at 60° C. for 1 h, and endogenous peroxidases were quenched with H₂O₂ for 10 min at room temperature. Slides were boiled for 15 min in RNAscope Target Retrieval Reagents and incubated for 30 min in RNAscope Protease Plus reagent prior to SARS-CoV2 RNA probe (Advanced Cell Diagnostics 848561) hybridization and signal amplification. Sections were counterstained with Gill's hematoxylin and visualized by brightfield microscopy. Some lung sections were processed for histology after hematoxylin and eosin staining.

SARS-CoV-2 Neutralization assays: Heat-inactivated serum samples were diluted serially and incubated with 10² FFU of SARS-CoV-2 for 1 h at 37° C. The virus-serum mixtures were added to Vero cell monolayers in 96-well plates and incubated for 1 h at 37° C. Subsequently, cells were overlaid with 1% (w/v) methylcellulose in MEM supplemented with 2% FBS. Plates were incubated for 30 h before fixation using 4% PFA in PBS for 1 h at room temperature. Cells were washed then sequentially incubated with anti-SARS-CoV-2 CR3022 antibody (Yuan et al., 2020) (1 pg/mL) and a HRP-conjugated goat anti-human IgG (Sigma) in PBS supplemented with 0.1% (w/v) saponin (Sigma) and 0.1% BSA. TrueBlue peroxidase substrate (KPL) was used to develop the plates before counting the foci on a BioSpot analyzer (Cellular Technology Limited). For neutralization experiments with luciferase expressing SARS-CoV-2, serum samples were diluted 3-fold starting at 1:50 and mixed with 85 PFU of each recombinant virus (wild-type and D614G). Vero E6 cells plated in clear-bottom black-walled 96-well plates (Corning) were inoculated with serum-virus mixtures, and cells were cultured at 37° C. for 48 h. Subsequently, cells were lysed and luciferase activity was measured using the Nano-Glo Luciferase Assay System (Promega) according to the manufacturer's specifications.

Hu-AdV5 neutralization assays: One day prior to Hu-AdV5-hACE2 transduction, serum samples were collected from mice immunized intramuscularly with ChAd-Control or ChAd-SARS-CoV-2-S. Sera were heat-inactivated and serially diluted prior to incubation with 10² FFU of HuAdV5 for 1 h at 37° C. The virus-serum mixtures were added to HEK293 cell monolayers in 96-well plates and incubated for 1 h at 37° C. Cells were then overlaid with 1% (w/v) methylcellulose in MEM supplemented with 5% FBS. Plates were incubated at 37° C. for 48 h before fixation with 2% PFA in PBS for 1 h at room temperature. Subsequently, plates were washed with PBS and incubated overnight at 4° C. with biotinylated anti-HuAdV5-hexon antibody (2 pg/mL; Novus Biologicals NB600413) diluted in permeabilization buffer (PBS supplemented with 0.1% (w/v) saponin and 0.1% BSA). Plates were washed again and incubated with streptavidin-HRP (1:3000; Vector Laboratories SA-5004) in permeabilization buffer for 30 min at room temperature. After a final wash series, plates were developed using TrueBlue peroxidase substrate (KPL) and foci were counted on a BioSpot analyzer (Cellular Technology Limited).

Protein expression and purification: Purified RNA from the 2019-nCoV/USA-WA1/2020 SARS-CoV-2 strain was reverse transcribed into cDNA and used as the template for recombinant gene cloning. A full-length SARS-CoV-2 NP (NP-FL) was cloned into pET21a with a hexahistidine tag and recombinantly expressed using BL21(DE3)-RIL E. coli in Terrific Broth (bioWORLD). Following overnight induction with isopropyl p3-d-1-thiogalactopyranoside (Goldbio) at 25° C., cells were lysed in 20 mM Tris-HCl pH 8.5, 1 M NaCl, 5 mM p3-mercaptopethanol, and 5 mM imidazole for nickel-affinity purification. Following elution in the prior buffer supplemented with 500 mM imidazole, the protein was purified to homogeneity using size exclusion and, in some cases, cation exchange chromatography. SARS-CoV-2 RBD and S ectodomain (S1/S2 furin cleavage site was disrupted, double proline mutations were introduced into the S2 subunit, and foldon trimerization motif was incorporated) were cloned into pFM1.2 with a C-terminal hexahistidine or octahistidine tag, transiently transfected into Expi293F cells, and purified by cobalt-charged resin chromatography (G-Biosciences) as previously described (Alsoussi et al., 2020).

ELISA: Purified antigens (S, RBD, or NP) were coated onto 96-well Maxisorp clear plates at 2 pg/mL in 50 mM Na₂CO₃ pH 9.6 (70 pL) overnight at 4° C. Coating buffers were aspirated, and wells were blocked with 200 μL of 1×PBS+0.05% Tween-20+1% BSA+0.02% NaN₃ (Blocking buffer, PBSTBA) either for 1 h at 37° C. or overnight at 4° C. Heat-inactivated serum samples were diluted in PBSTBA in a separate 96-well polypropylene plate. The plates then were washed thrice with 1×PBS +0.05% Tween-20 (PBST), followed by addition of 50 μL of respective serum dilutions. Sera were incubated in the blocked ELISA plates for at least 1 h at room temperature. The ELISA plates were again washed thrice in PBST, followed by addition of 50 μL of 1:2000 anti-mouse IgG-HRP (Southern Biotech Cat. #1030-05) in PBST or 1:10000 of biotinylated anti-mouse IgG, anti-mouse IgM, or anti-mouse IgA in PBSTBA (SouthernBiotech). Plates were incubated at room temperature for 1 h, washed thrice in PBST, and then 1:5000 dilution of streptavidin-HRP (ThermoFisher) was added to wells. Following a 1 h incubation at room temperature, plates were washed thrice with PBST and 50 μL of 1-Step Ultra TMB-ELISA was added (ThermoFisher Cat. #34028). Following a 12 to 15-min incubation, reactions were stopped with 50 μL of 2 M sulfuric acid. The absorbance of each well at 450 nm was read (Synergy H1) within 2 min of addition of sulfuric acid. Optical density (450 nm) measurements were determined using a microplate reader (Bio-Rad).

EL/Spot assay: MultiScreen-HA filter 96-well plates (Millipore) plates were pre-coated with 3 pg/ml of SARS-CoV-2 S protein overnight at 4° C. After rinsing with PBST, plates were blocked for 4 h at 37° C. with culture medium (RPMI, 10% FBS, penicillin-streptomycin, 1 mM sodium pyruvate, 0.1 mM non-essential amino acids, 10 mM HEPES, and 50 mM p-mercaptoethanol). Single cell suspensions of splenocytes in culture medium were added to the S protein coated plates and incubated at 37° C. and 5% humidified CO₂ for 4 h. After washing with PBS and PBST, plates were incubated with biotinylated anti-IgG or anti-IgA (Southern Biotech) followed by incubation with streptavidin conjugated horseradish peroxidase (Jackson ImmunoResearch), each for 1 h at room temperature. After additional washes with PBS, 3-amino-9-ethylcarbazole (Sigma) substrate solution was added for spot development. The reaction was stopped by rinsing with water. Spots were counted using a Biospot plate reader (Cellular Technology).

Measurement of viral burden: SARS-CoV-2 infected mice were euthanized using a ketamine and xylazine cocktail, and organs were collected. Tissues were weighed and homogenized with beads using a MAgNA Lyser (Roche) in 1 ml of Dulbecco's Modified Eagle's Medium (DMEM) containing 2% fetal bovine serum (FBS). RNA was extracted from clarified tissue homogenates using MagMax mirVana Total RNA isolation kit (Thermo Scientific) and a Kingfisher duo prime extraction machine (Thermo Scientific). SARS-CoV-2 RNA levels were measured by one-step quantitative reverse transcriptase PCR (qRT-PCR) TaqMan assay as described previously (Hassan et al., 2020). SARS-CoV-2 nucleocapsid (N) specific primers and probe set were used: (L Primer: ATGCTGCAATCGTGCTACAA (SEQ ID NO: 8); R primer: GACTGCCGCCTCTGCTC (SEQ ID NO: 9). Viral RNA was expressed as (N) gene copy numbers per milligram on a log 10 scale. For some samples, viral titer was determined by plaque assay on Vero E6 cells. Cytokine and chemokine mRNA measurements. RNA extracted from lung homogenates lung homogenates was DNAse-treated and used to synthesize cDNA using the High-Capacity cDNA Reverse Transcription kit (Thermo Scientific) with the addition of RNase inhibitor according to the manufacturer's protocol. Cytokine and chemokine expression was determined using TaqMan Fast Universal PCR master mix (Thermo Scientific) with commercial primers/probe sets specific for IFN-γ (IDT: Mm.PT.58.41769240), IL-6 (Mm.PT.58.10005566), IL-1□ (Mm.PT.58.41616450), TNF-α (Mm.PT.58.12575861), CXCL10 (Mm.PT.58.43575827), CCL2 (Mm.PT.58.42151692), CCL5 (Mm.PT.58.43548565), CXCL11 (Mm.PT.58.10773148.g), IFN-β (Mm.PT.58.30132453.g), and IFNγ-2/3 (Thermo Scientific Mm04204156_gH) and results were normalized to GAPDH (Mm.PT.39a.1) levels. Fold change was determined using the 2^{-ΔΔCt} method comparing treated mice to naïve controls.

Peptide restimulation and intracellular cytokine staining: Splenocytes from intramuscularly vaccinated mice were incubated in culture with a pool of 253 overlapping 15-mer SARS-CoV-2 S peptides for 12 h at 37° C. before a 4 h treatment with brefeldin A (BioLegend, 420601). Following blocking with FcγR antibody (BioLegend, clone 93), cells were stained on ice with CD45 BUV395 (BD BioSciences clone 30-F11); CD44 PE-Cy7, CD4 PE-Cy5, CD8b PreCP-Cy5.5, and CD19 APC-Cy7 (BioLegend clones, IM7, GK1.5, YTS156.7.7, and 6D5, respectively), and Fixable Aqua Dead Cell Stain (Invitrogen, L34966). Stained cells were fixed and permeabilized with the Foxp3/Transcription Factor Staining Buffer Set (eBiosciences, 00-5523). Subsequently, intracellular staining was performed with anti-IFN-γ Alexa 647 (BD Biosciences, clone XMG1.2), anti-TNFα BV605 (BioLegend, clone MP6-XT22) and anti-GrB PE (Invitrogen, GRB04). Lungs from intranasally immunized mice were harvested and digested for 1 h at 37° C. in digestion buffer consisting of RPMI media supplemented with (167 μg/ml) of Liberase DH (Sigma) and (100 □g/ml) of DNase I (Sigma). Lung cells were incubated at 37° C. with the pool of 253 overlapping 15-mer SARS-CoV-2 S peptides described above in the presence of brefeldin A for 5 h at 37° C. Lung cells then were stained as described above except that CD4-BV421 (BioLegend clone GK1.5) replaced the CD4-PE-Cy5, no CD19 staining was included, and CD103-FITC and CD69-BV711 (BioLegend clones, 2E7, and, H1.2F3, respectively) were added. Analysis was performed on a BD LSRFortessa X-20 cytometer, using FlowJo X 10.0 software.

Flow cytometry-based antigen characterization: HEK-293T cells were seeded at 10⁶ cells/well in 6-well plates 24 h prior to transduction with ChAd-SARS-CoV-2-S (MOI of 5). After 20 h, cells were harvested, fixed and permeabilized using Foxp3 Transcription Factor Staining Buffer Set (Thermo Fisher), and stained for viral antigen after incubation with the following anti-SARS-CoV-2 neutralizing murine mAbs: SARS2-01, SARS2-02, SARS2-07, SARS2-11, SARS2-12, SARS2-16, SARS2-18, SARS2-20, SARS2-21, SARS2-22, SARS2-23, SARS2-29, SARS2-31, SARS2-32, SARS2-34, SARS2-38, SARS2-39, SARS2-50, SARS2-55, SARS2-58, SARS2-66, and SARS2-71 (L. VanBlargan and M. Diamond, unpublished results). H77.39, an isotype-matched anti-HCV E2 mAb was used as a negative control. Cells were washed, incubated with Alexa Fluor 647 conjugated goat anti-mouse IgG (Thermo Fisher), and analyzed by flow cytometry using a MACSQuant Analyzer 10 (Miltenyi Biotec). The percentage of cells positive for a given mAb was compared with cells stained with a saturating amount of an oligoclonal mixture of anti-SARS-CoV-2 mAbs.

### Example 2: An Intranasal Vaccine Durably Protects Against SARS-CoV-2 Variants in Mice

SARS-CoV-2 variants that attenuate antibody neutralization could jeopardize vaccine efficacy and the end of the COVID-19 pandemic. Example 1 shows the protective activity of a single-dose intranasally-administered spike protein-based chimpanzee adenovirus-vectored vaccine (ChAd-SARS-CoV-2-S) in animals, which has advanced to human trials. The present example provides the durability, dose-response, and cross-protective activity in mice. A single intranasal dose of ChAd-SARS-CoV-2-S induced durably high neutralizing and Fc effector antibody responses in serum and S-specific IgG and IgA secreting long-lived plasma cells in the bone marrow. Protection against a historical SARS-CoV-2 strain was observed across a 100-fold vaccine dose range and over a 200-day period. At 6 weeks or 9 months after vaccination, serum antibodies neutralized SARS-CoV-2 strains with B.1.351 and B.1.1.28 spike proteins and conferred almost complete protection in the upper and lower respiratory tracts after challenge. Thus, in mice, intranasal immunization with ChAd-SARS-CoV-2-S provides durable protection against historical and emerging SARS-CoV-2 strains.

The spike (S) protein of the SARS-CoV-2 virion is the principal target for antibody-based and vaccine countermeasures. The S protein serves as the primary viral attachment and entry factor and engages the cell-surface receptor angiotensin-converting enzyme 2 (ACE2) to promote SARS-CoV-2 entry into human cells. SARS-CoV-2 S proteins are cleaved to yield S1 and S2 fragments, with the S1 protein containing the receptor binding domain (RBD) and the S2 protein promoting membrane fusion and virus penetration into the cytoplasm. The prefusion form of the SARS-CoV-2 S protein is recognized by potently neutralizing monoclonal antibodies or protein inhibitors.

Many vaccine candidates targeting the SARS-CoV-2 S protein have been developed using DNA plasmid, lipid nanoparticle encapsulated mRNA, inactivated virion, protein subunit, or viral-vectored vaccine platforms. Several vaccines administered by intramuscular (IM) injection (e.g., Pfizer/BioNTech BNT162b2 and Moderna 1273 mRNA and Johnson & Johnson Ad26.COV2 and AstraZeneca ChAdOx1 nCoV-19 adenoviral platforms) have been granted emergency use authorization in many countries with hundreds of million of doses given worldwide (covid19.who.int).

While vaccines administered by IM injection induce robust systemic immunity that protects against severe disease and mortality, questions remain as to their ability to curtail SARS-CoV-2 transmission, especially if upper airway infection is not reduced. Indeed, many of the IM-administered vaccines showed variable protection against upper airway infection and transmission in pre-clinical studies and failed to induce substantive mucosal (IgA) immunity. This issue is important because of the emergence of more transmissible SARS-CoV-2 variants including B.1.1.7, B.1.351, and B.1.1.28 with substitutions in the spike protein. Experiments with pseudoviruses and authentic SARS-CoV-2 strains also suggest that neutralization by vaccine-induced sera is diminished against variants expressing mutations in the spike gene at positions L452, E484, and elsewhere. Beyond possible negative impacts on protection, the combination of diminished immunity against certain variants and naturally lower anti-S IgG levels in the respiratory mucosa could create conditions for further selection of resistance in the upper airway and transmission into the general population.

As described herein, a single-dose, intranasally (IN)-delivered chimpanzee Adenovirus (simian Ad-36)-based SARS-CoV-2 vaccine (ChAd-SARS-CoV-2-S) encoding a pre-fusion stabilized S protein that induced robust humoral, cell-mediated, and mucosal immune responses and limited upper and lower airway infection in K18-hACE2 transgenic mice, hamsters, and non-human primates. This vaccine, which has advanced to human clinical trials (BBV154, Clinical Trial NCT04751682), differs from ChAdOx1 nCoV-19, a chimpanzee Ad-23-based SARS-CoV-2 vaccine, currently granted emergency use in some countries. Here, as a further step to evaluating the potential utility of ChAd-SARS-CoV-2-S, its dose-response, durability, and cross-protective activity in mice including effects on upper and lower airway infection was assessed. At approximately nine months after IN immunization, neutralizing antibody and anti-S protein IgA levels in serum of ChAd-SARS-CoV-2-S-vaccinated animals remained high and inhibited infection with SARS-CoV-2 strains with B.1.351 and B.1.1.28 spike proteins. At this time, susceptible K18-hACE2 transgenic mice were fully protected against upper and lower respiratory tract infection after challenge with a SARS-CoV-2 virus displaying B.1.351 spike proteins.

Results

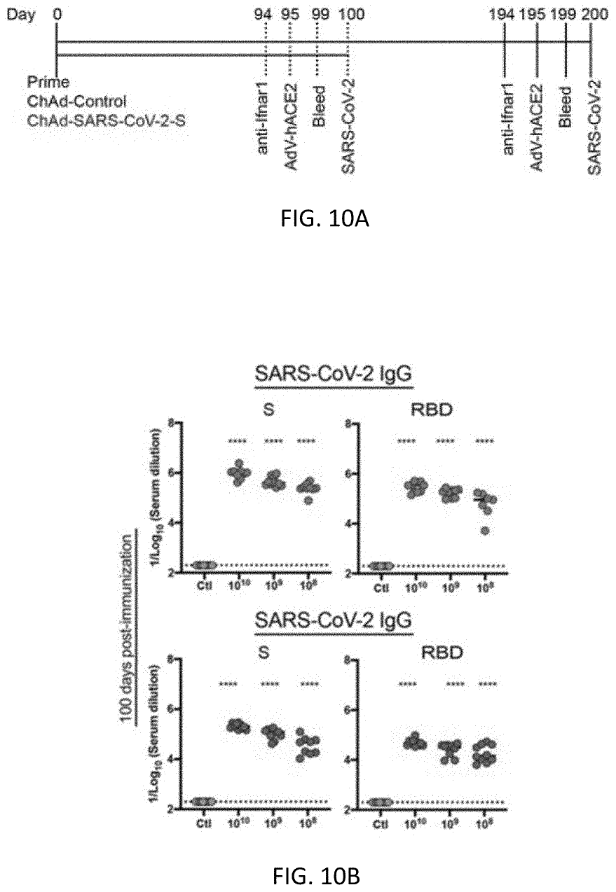

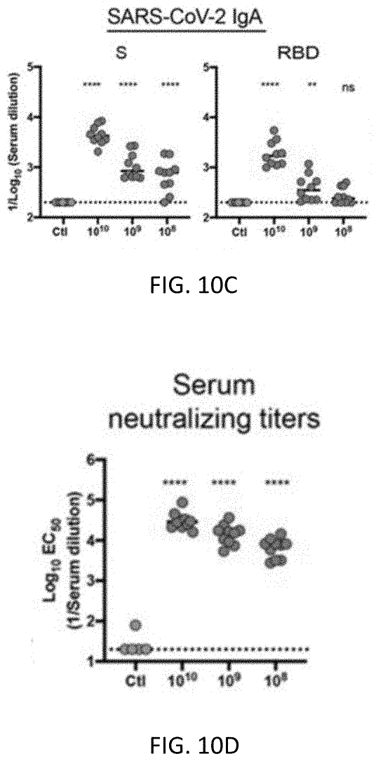

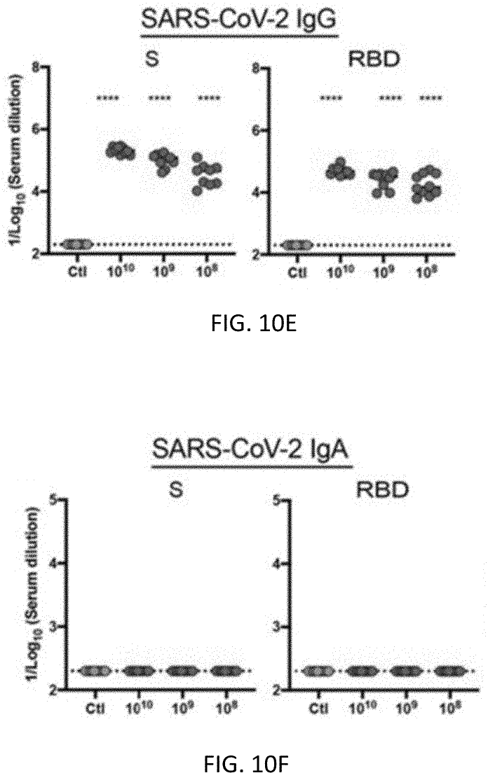

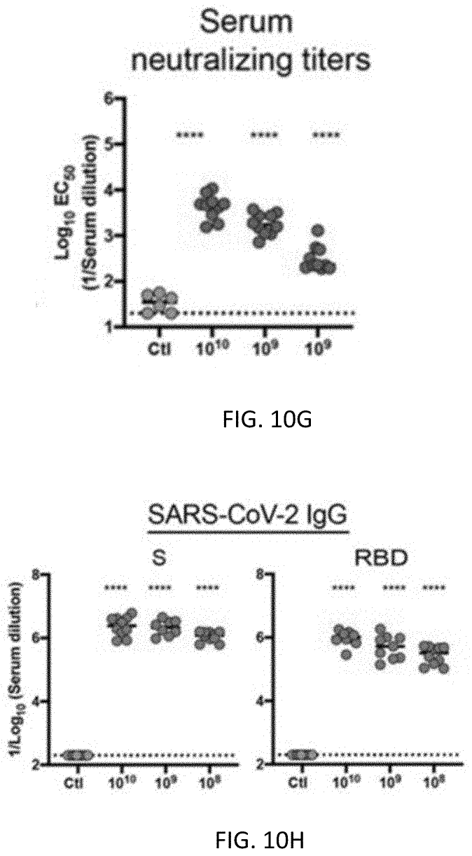

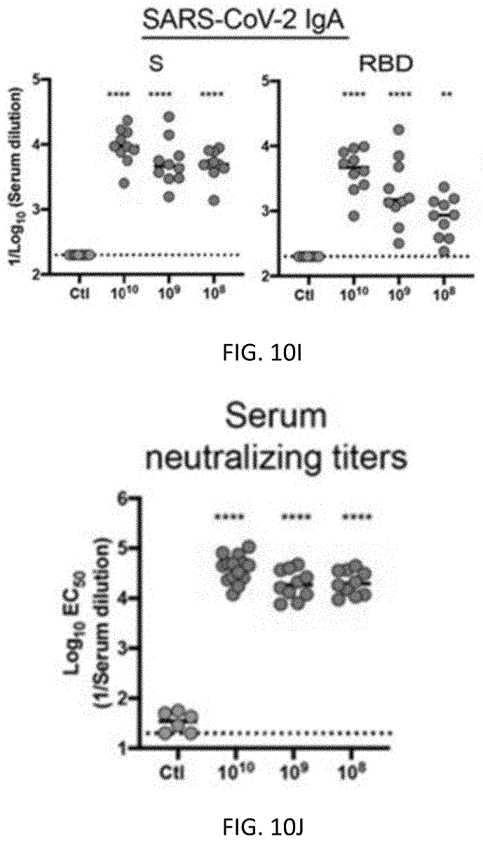

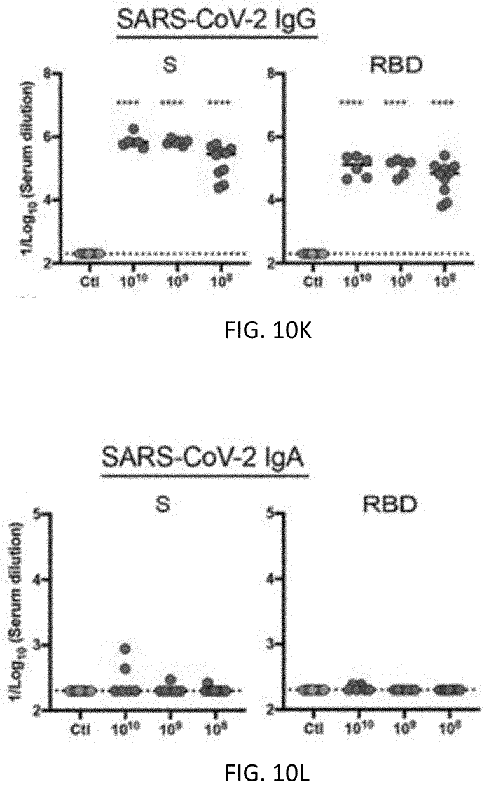

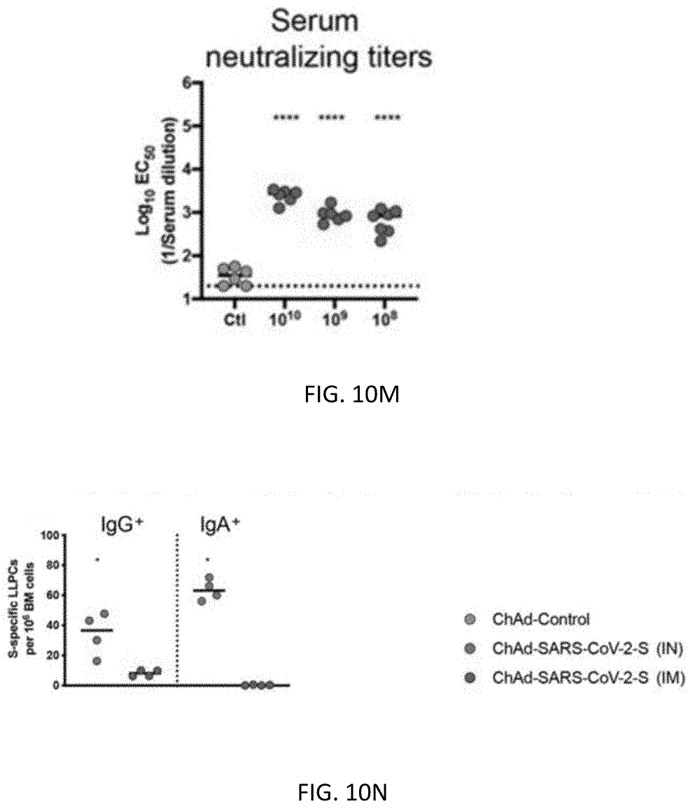

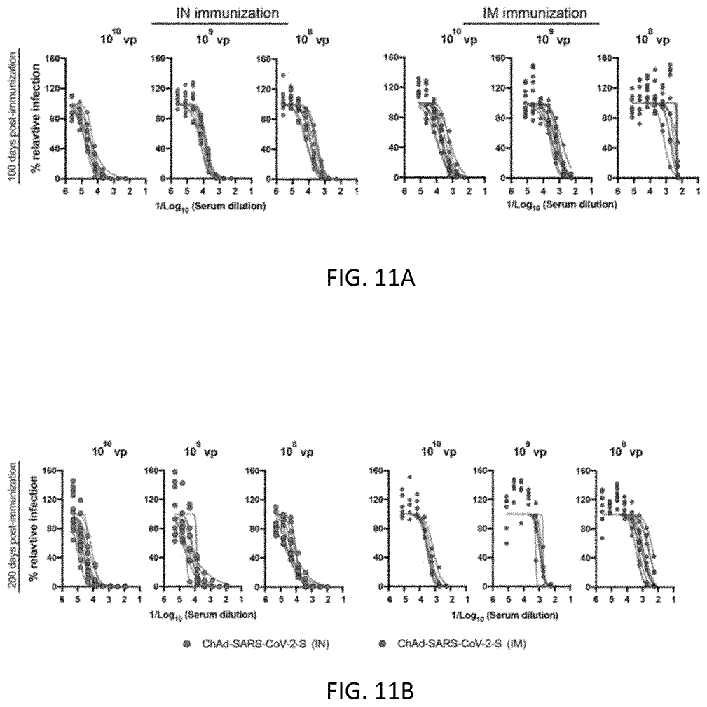

A single ChAd-SARS-CoV-2-S immunization induces durable anti-spike and neutralizing responses at different doses: The durability of humoral immune responses in BALB/c mice 100 or 200 days post IM- or IN-immunization with escalating doses of ChAd-SARS-CoV-2-S (10⁸, 10⁹, and 10¹⁰ viral particles [vp]) or 10¹⁰ vp of a ChAd-Control vaccine was assessed (FIG. 10A). First, anti-S and anti-RBD IgG and IgA levels were measured by ELISA. Consistent with prior results at a one-month time point 30, at 100 or 200 days post-vaccination, IN immunization with ChAd-SARS-CoV-2-S induced superior antibody responses than IM immunization or vaccination with ChAd-Control (FIG. 10B-10M and FIG. 11). Anti-S and anti-RBD-specific binding IgG levels in serum were greater after IN than IM immunization at 100 or 200 days. At 100 days after IN immunization with 10¹⁰, 10⁹, and 10⁸ vp of ChAd-SARS-CoV-2-S, geometric mean titers (GMT) of S-specific IgG responses were 1.1×10⁶, 4.8×10⁵, and 2.6×10⁵, and RBD-specific IgG were 3.2×10⁵, 1.8×10⁵, and 8.7×10⁴, respectively (FIG. 10B). In comparison, S- and RBD-specific IgG responses 100 days after IM immunization with 10¹⁰, 10⁹, and 10⁸ vp of ChAd-SARS-CoV-2-S were 4 to 6-fold lower (P<0.0001) with S-specific IgG titers of 2.1×10⁵, 1.1×10⁵, and 4.5×10⁴ and RBD-specific IgG titers of 5.1×10⁴, 2.9×10⁴, and 2.3×10⁴, respectively (FIG. 10E). A similar dose response was observed with S- and RBD-specific IgG titers at 200 days after IN or IM immunization (FIG. 10H and FIG. 10K). At 200 days after IN immunization with 10¹⁰, 10⁹, and 10⁸ vp of ChAd-SARS-CoV-2-S, GMT of S-specific IgG were 2.8×10⁶, 2.4×10⁶, and 1.2×10⁶, and RBD-specific IgG were 1.1×10⁶, 6.1×10⁵, and 3.2×10⁵, respectively (FIG. 10H). At 200 days after IM immunization with 10¹⁰, 10⁹, and 10⁸ vp of ChAd-SARS-CoV-2-S, S-specific IgG GMT were 8.1×10⁵, 6.9×10⁵, and 2.6×10⁵, and RBD-specific IgG GMT were 1.4×10⁵, 1.3×10⁵, and 8.0×10⁴, respectively (FIG. 10K). Thus, anti-S and anti-RBD IgG levels were higher after IN than IM immunization and continued to rise in serum even several months after single-dose vaccination.

We next assessed the induction and durability of serum IgA responses. Although IM immunization failed to induce S- or RBD-specific IgA (FIGS. 1F and L), substantial levels of anti S- and RBD IgA were detected after IN immunization at 100 or 200 days post-immunization (FIGS. 1C and I). At 100 days after IN immunization with 1010, 109, and 108 vp of ChAd-SARS-CoV-2-S, the GMT of S-specific IgA were 4.8×103, 1.2×103, and 8.4×102, and RBD-specific IgA were 2.2×103, 4.6×102, and 2.9×102, respectively (FIG. 1C). As seen with IgG, the IgA levels continued to increase over time such that at 200 days the GMT of S-specific IgA were 1.1×104, 7.4×103, and 5.4×103, and RBD-specific IgA were 5.2×103, 3.8×103, and 9.8×102 after IN immunization with 1010, 109, and 108 vp of ChAd-SARS-CoV-2-S, respectively (FIG. 1I).

Next, a functional correlate of the serological response was evaluated by assaying neutralizing activity (FIG. 10D, FIG. 10G, FIG. 10J, FIG. 10M and FIG. 11) using a focus-reduction neutralization test (FRNT). As expected, neutralizing activity was not detected in sera from ChAd-control treated mice. At 100 days post IN immunization with 10¹⁰, 10⁹, and 10⁸ vp of ChAd-SARS-CoV-2-S, the mean effective half maximal inhibitory titers [EC50] were 39,449, 9,989, and 7,270, respectively (FIG. 10D). In comparison, at this time point after IM immunization with 10¹⁰, 10⁹, and 10⁸ vp of ChAd-SARS-CoV-2-S, the EC50 values were 8 to 20-fold lower (P<0.0001) at 4,988, 2,017, and 391, respectively (FIG. 10G). At 200 days after IN immunization with 10¹⁰, 10⁹, and 10⁸ vp of ChAd-SARS-CoV-2-S, and consistent with the higher anti-S and RBD titers seen, EC50 values were 45,591, 22,769, and 23,433, respectively (FIG. 10J). In comparison, 200 days after IM immunization with 10¹⁰, 10⁹, and 10⁸ vp of ChAd-SARS-CoV-2-S, EC50 values were much lower at 2,524, 940, and 716, respectively (FIG. 10M).

Long-lived plasma cells (LLPCs) reside in the bone marrow and constitutively secrete high levels of antibody that correlate with serum levels. To assess the levels of antigen-specific LLPCs at 200 days after IM or IN immunization with 10¹⁰ vp of ChAd-SARS-CoV-2-S, CD138⁺ cells were isolated from the bone marrow and assayed for S-specific IgG or IgA production using an ELISPOT assay. A ˜4-fold higher frequency of LLPCs secreting S-specific IgG after IN immunization than IM immunization was observed (FIG. 10N). Additionally, after IN immunization, higher numbers of LLPCs producing S-specific IgA were detected, which were absent after IM immunization (FIG. 10N). Together, these data establish the following: (a) single-dose IN immunization promotes superior humoral immunity than IM immunization; (b) 100-fold lower inoculating doses of ChAd-SARS-CoV-2-S induce robust neutralizing antibody responses in mice; (c) IN but not IM immunization induces serum IgA responses and IgA-specific LLPCs against the SARS-CoV-2 S protein; and (d) the humoral immunity induced by ChAd-SARS-CoV-2-S is durable and rises over a six-month period after vaccination.

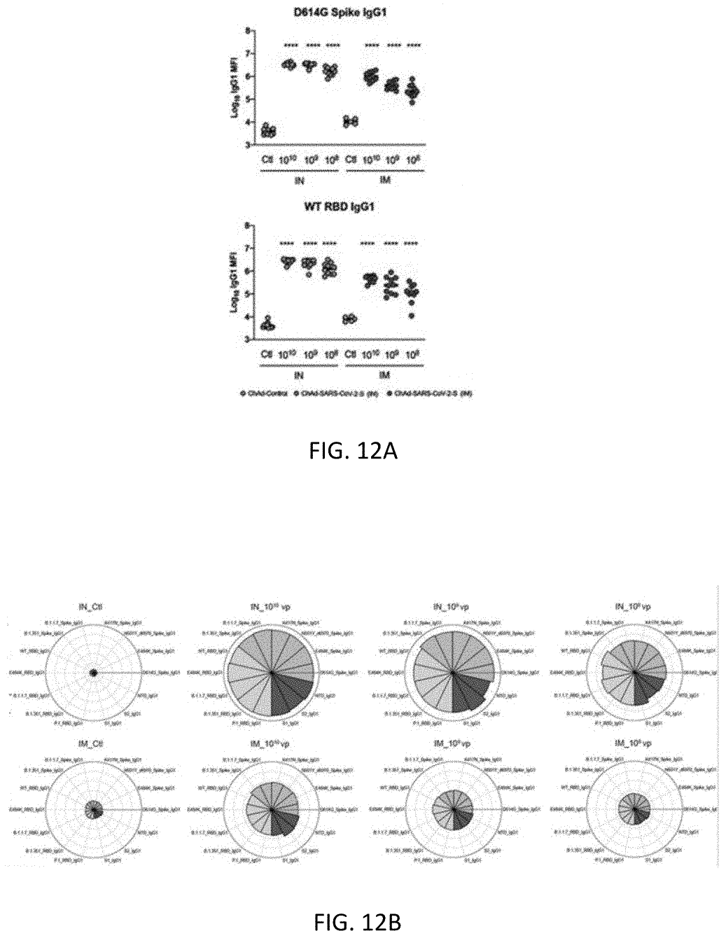

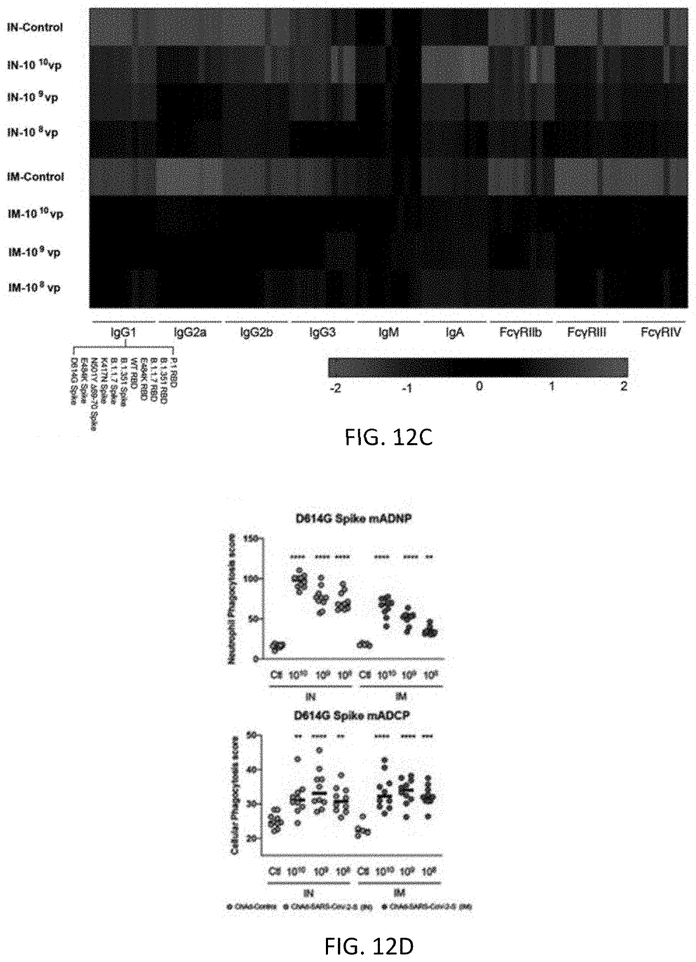

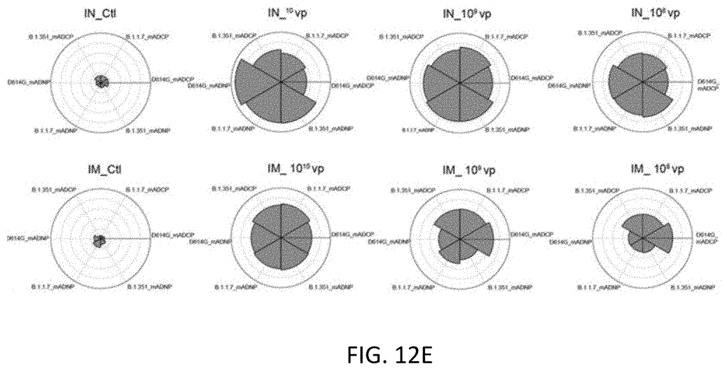

IN inoculation of ChAd-SARS-CoV-2-S induces broad antibody responses with Fc effector function capacity: To characterize the humoral response further, antibody binding to SARS-CoV-2 variant proteins and Fc effector functions using serum derived from BALB/c mice were analyzed at 90 days after IN or IM vaccination. Our panel of SARS-CoV-2 proteins included spike (D614G, E484K, N501Y, Δ69-70, K417N) and RBD (E484K) antigens corresponding to WA1/2020, B.1.1.7, B.1.351, B.1.1.28 strains. First, the anti-SARS-CoV-2 specific antibody response for several isotypes (IgG1, IgG2a, IgG2b, IgG3, IgM, and IgA) and their ability to bind Fcγ receptors (mouse FcγRIIB, FcγRIII, FcγRIV) using a luminex platform were measured. Consistent with data obtained by ELISA (FIG. 10B and FIG. 10E), IN vaccination of ChAd-SARS-CoV-2-S induced higher levels of IgG1 to D614G spike and WA1/2020 RBD proteins than IM immunization, and as expected, decreasing doses of the vaccine elicited lower antibody titers (FIG. 12A). Anti-SARS-CoV-2 IgG1 titers after IN immunization also were higher against all spike and RBD variants than after IM immunization, and titers decreased with vaccine dose (FIG. 12B). As shown in a heatmap, this trend was observed for all anti-SARS-CoV-2 specific antibody isotypes and correlated with FcγR binding patterns (FIG. 12C). These data suggest that IN vaccination induces a higher magnitude and broader antibody subclass response to SARS-CoV-2 than IM vaccination.

Antibody effector functions, such as opsonization, are mediated in part by Fcγ receptor engagement. To determine if the observed differences in antibody titers and FcγR binding titers resulted in differences in effector functions, antibody-dependent neutrophil (ADNP) and cellular phagocytosis (ADCP) assays were performed (FIG. 12D-12E). Sera from IN vaccinated mice stimulated substantially more ADNP than those obtained from IM vaccinated mice. However, minimal differences in ADCP were apparent from antibodies derived after IN and IM vaccination (FIG. 12D-12E). These data demonstrate that IN vaccination with ChAd-SARS-CoV-2-S induces a greater and more functional antibody response than after IM vaccination.

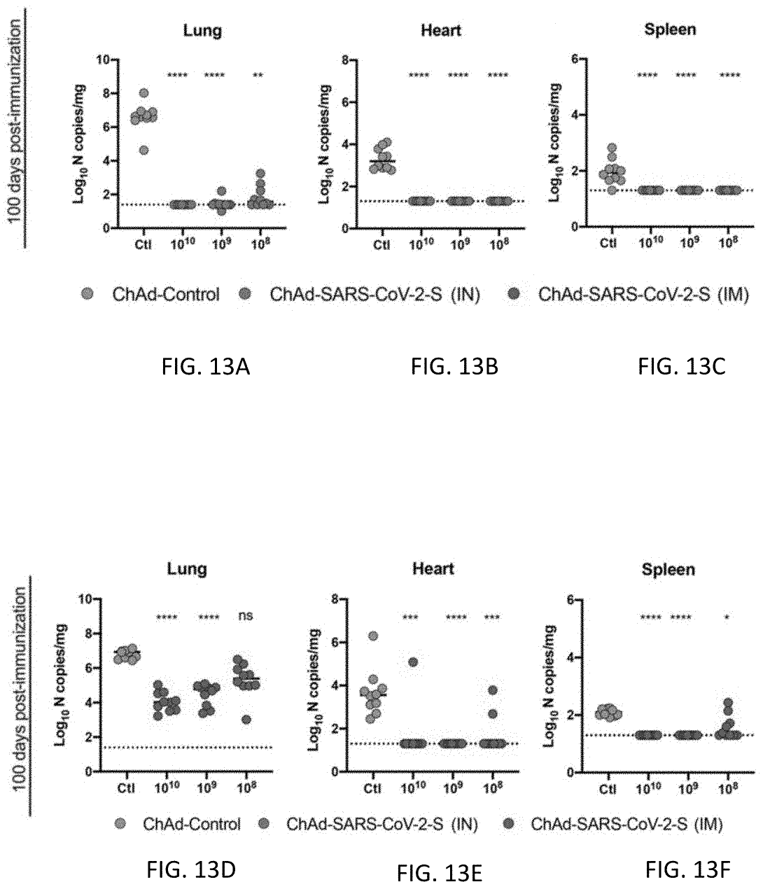

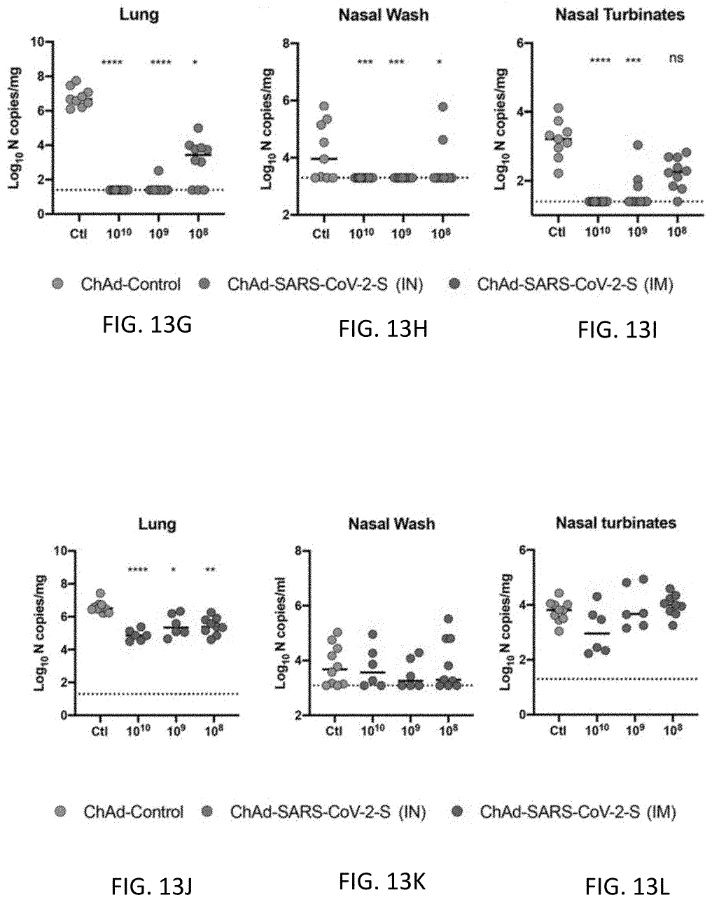

Intranasally administered ChAd-SARS-CoV-2-S induces durable protection against SARS-CoV-2 challenge in BALB/c mice: To assess the efficacy of the ChAd-SARS-CoV-2-S vaccine, immunized BALB/c mice given the dosing regimen described in FIG. 10A were challenged with SARS-CoV-2. Virus challenge was preceded by intransal introduction of Hu-Ad5-hACE2, which enables ectopic expression of hACE2 and productive infection of SARS-CoV-2 in BALB/c mice by historical SARS-CoV-2 strains. Animals were immunized once via IN or IM routes with 10¹⁰ vp of ChAd-Control or 10⁸, 10⁹, or 10¹⁰ vp of ChAd-SARS-CoV-2-S. At day 95 or 195 post-vaccination, mice were given 10⁸ plaque-forming units (PFU) of Hu-Ad5-hACE2 and anti-Ifnar1 mAb; the latter attenuates innate immunity and enhances pathogenesis in this model. Five days later, BALB/c mice were challenged with 5×10⁴ focus-forming units (FFU) of SARS-CoV-2 (strain WA1/2020) via IN route. At 4 days post-infection (dpi), lungs, spleen, and heart were harvested from mice challenged at 100 days post-immunization, and lungs, nasal turbinates, and nasal washes were collected from a second cohort challenged at 200 days post-immunization. Tissues were assessed for viral burden by quantitative reverse transcription PCR (qRT-PCR) using primers for the subgenomic RNA (N gene). IN immunization with all three doses induced remarkable protection at 100 days post-vaccination as evidenced by a virtual absence of viral RNA in lungs, spleen, and heart compared to animals receiving the ChAd-Control vaccine (FIG. 13A-13C). At 200 days post-immunization, protection conferred by the IN delivered ChAd-SARS-CoV-2-S remained robust in the upper and lower respiratory tracts compared to ChAd-Control immunized mice. Nevertheless, limited infection breakthrough in the lungs and nasal turbinates in animals immunized with the lowest 10⁸ vp dose of ChAd-SARS-CoV-2-S was observed (FIG. 13G and FIG. 131). In comparison, protection at 100 days post-IM immunization was less than after IN immunization at the same challenge time point. Although viral RNA was not detected in the heart and spleen (FIG. 13E-13F), at least 1,000 to 30,000-fold (P<0.0001) higher levels were measured in the lungs of mice immunized with ChAd-SARS-CoV-2-S by the IM compared to IN route (FIG. 13A and FIG. 13D). A greater impact of dosing by the IM route was also observed, as the reduction in viral RNA load in the lung at 10⁸ vp dose no longer was different than in the ChAd-Control vaccinated mice (FIG. 13D). At 200 days post-IM immunization, less protection against SARS-CoV-2 infection in the lungs, nasal washes, and nasal turbinates was observed than after IN immunization (FIG. 13G-13L).

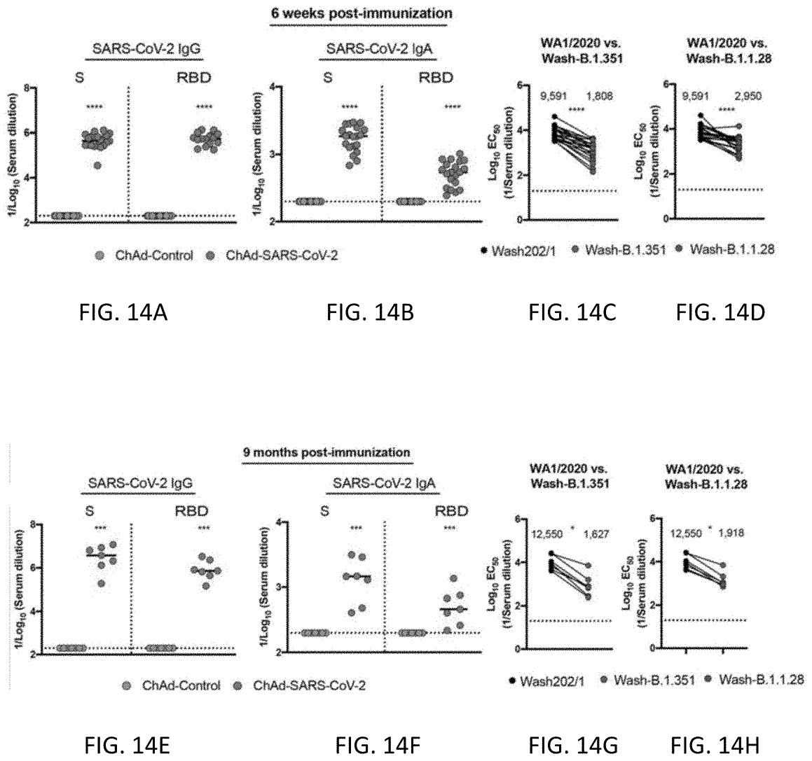

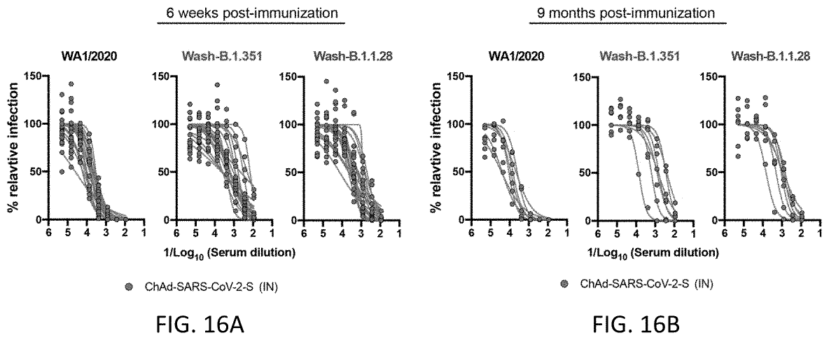

ChAd-SARS-CoV-2-S induces durable immunity in hACE2 transgenic mice: Next, the immunogenicity of intransally-delivered ChAd-SARS-CoV-2-S in K18-hACE2 C57BL/6 mice was assessed, which are more vulnerable to SARS-CoV-2 infection than BALB/c mice. Five-week old K18-hACE2 mice were inoculated via an IN route with 109 vp of ChAd-control or ChAd-SARS-CoV-2-S. Serum samples were collected six weeks later, and humoral immune responses were evaluated. IN immunization of ChAd-SARS-CoV-2-S but not ChAd-control induced high levels of S- and RBD-specific IgG and IgA (FIG. 14A-14B). Neutralizing antibody titers against WA1/2020 and two other SARS-CoV-2 strains with spike proteins from B.1.351 and B.1.1.28 variants were measured by FRNT assay (FIG. 14C-14D and FIG. 16). High levels of neutralizing antibody against WA1/2020 were induced after a single IN dose of ChAd-SARS-CoV-2-S (EC50 of 9,591). As seen with vaccine-induced human sera, decreases in neutralizing titers were observed against Wash-B.1.351 (˜5-fold, P<0.0001; FIG. 14C) and Wash-B.1.1.28 (˜3-fold, P<0.0001; FIG. 4D) SARS-CoV-2 strains compared to WA1/2020. To assess the durability of humoral responses, a separate cohort of K18-hACE2 mice were immunized via the IN route, and serum samples were collected at nine months. ChAd-SARS-CoV-2-S induced high levels of S- and RBD-specific IgG and IgA and neutralizing antibody against WA1/2020 (EC50 of 12,550) at this time point (FIG. 14E-14H and FIG. 16). When tested against the Wash-B.1.351 and Wash-B.1.1.28 viruses, a decrease in neutralizing titer was also observed (˜6 to 8-fold, P<0.05; FIG. 14G-14H) compared to WA1/2020, although they still remained high (EC50 of 1,627 and 1,918, respectively).

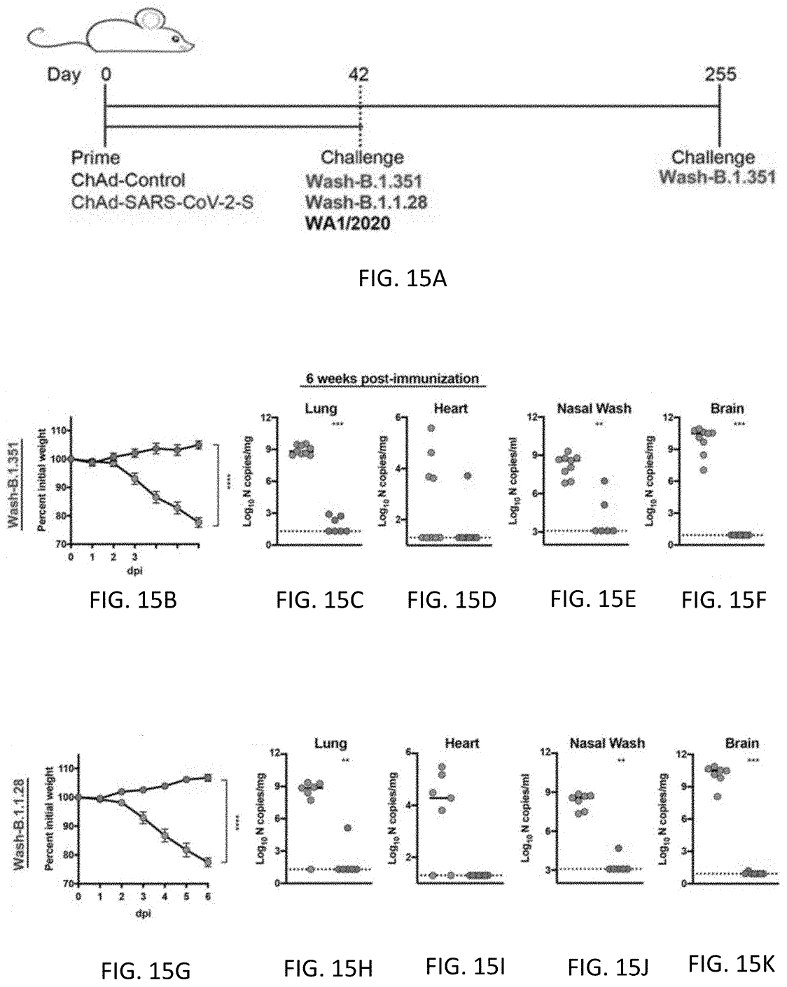

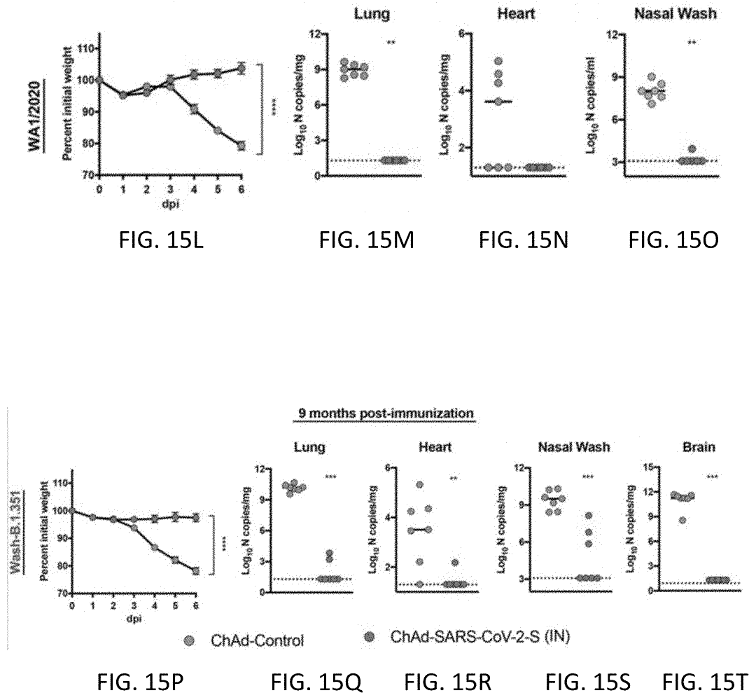

ChAd-SARS-CoV-2-S confers cross-protection against Wash B.1.351 and Wash-B.1.1.28 challenge in hACE2 transgenic mice: The protective efficacy of ChAd-SARS-CoV-2-S against WA1/2020 and two chimeric viruses (Wash-B.1.351 and Wash-B.1.1.28) with spike genes corresponding to variants of concern were tested (FIG. 15A). Five-week old K18-hACE2 mice were immunized via an IN route with a single 109 vp dose of ChAd-control or ChAd-SARS-CoV-2-S. Six weeks later, mice were challenged by an IN route with 104 FFU of Wash-B.1.351, Wash B.1.1.28, or WA1/2020. All mice immunized with ChAd-SARS-CoV-2 exhibited no weight loss, whereas most ChAd-Control-vaccinated mice experienced substantial weight loss at 3 to 6 dpi (FIG. 15B, FIG. 15G, and FIG. 15L). Remarkably, vaccination with ChAd-SARS-CoV-2-S resulted in almost no detectable SARS-CoV-2 RNA in the upper and lower respiratory tract, heart, and brain at 6 dpi (FIG. 15C-15F, FIG. 15H-15K, and FIG. 15M-15O). As a further test of the durability of the cross-protective response, five-week old K18-hACE2 mice were immunized via an IN route with a single 10¹⁰ vp dose of ChAd-control or ChAd-SARS-CoV-2-S. Nine months later, mice were challenged via IN route with 10⁴ FFU of Wash-B.1.351. ChAd-SARS-CoV-2-S-vaccinated mice maintained weight in contrast to ChAd-Control treated mice (FIG. 15P). Moreover, substantial virological protection was observed, as only very low amounts of Wash-B.1.351 SARS-CoV-2 RNA were detected in the upper and lower respiratory tracts, heart, and brain in some of the mice (FIG. 15Q-15T).

Discussion

The durability of vaccine-induced immune responses is a key for providing sustained protection against SARS-CoV-2 infection and curtailing the current pandemic. Here, it was shown that a single IN immunization with ChAd-SARS-CoV-2-S induced S- and RBD-specific binding and neutralizing antibodies that continued to rise for several months, suggestive of sustained germinal center reactions. LLPCs in the bone marrow were detected six months after IN vaccination, secreting SARS-CoV-2-specific IgG and IgA that likely contributed to the durably high antiviral antibody levels in circulation 34. In comparison, IM immunization with ChAd-SARS-CoV-2-S induced lower levels of serum neutralizing antibodies, fewer spike-specific IgG secreting LLPCs, and virtually no serum or cellular IgA response. At least in mice, a single IN dose immunization with ChAd-SARS-CoV-2-S produced durable humoral immunity that was observed across a 100-fold dose range. These pre-clinical immunogenicity results compare favorably with studies in humans with mRNA vaccines against SARS-CoV-2, which show humoral immune responses lasting at least several months. In comparison, the durability of antibody responses after natural SARS-CoV-2 infection can vary considerably.

A single immunization of ChAd-SARS-CoV-2-S conferred durable protection against SARS-CoV-2 (WA1/2020 strain) challenge in hACE2-transduced BALB/c mice or K18-hACE2 transgenic C57BL/6 mice at multiple time points through six months. IN immunization in particular provided virtually complete virological protection against upper and lower respiratory tract infection, with only a limited infection breakthrough seen at the 100-fold lower vaccine dose. The abrogation of infection in the upper respiratory tract suggests that IN vaccination could prevent transmission. In comparison, IM immunization reduced the viral RNA levels in the lungs but showed substantially less protection against the homolgous WA1/2020 strain in samples from the upper respiratory tract. While many SARS-CoV-2 vaccine candidates from different platforms have demonstrated immunogenicity and protective efficacy in animals models, to our knowledge, none have established durability or protection against variant viruses. The long-term protection conferred by IN immunization even at 100-fold lower inoculating doses is promising. If results in mice were recapitulated, dose sparing strategies could enable production of a large number of vaccine doses that could curtail infection and transmission of SARS-CoV-2.

The emergence of SARS-CoV-2 S variants with mutations of amino acids in the receptor binding motif (e.g., B.1.351 and B.1.1.28) is of concern because of their resistance to the inhibitory activity of many neutralizing antibodies. Indeed, human sera from subjects vaccinated with BNT162b2 mRNA or ChAdOx1 nCoV-19 (AZD1222) vaccines showed reduced neutralization against B.1.351. Concerningly, IM-administered ChAdOx1 nCoV-19 (AZD1222) showed reduced protective efficacy against mild to moderate B.1.351 infection in humans. In K18-hACE2 transgenic mice, when we compared the immunogenicity of IN-delivered ChAd-SARS-CoV-2-S against WA1/2020 and chimeric SARS-CoV-2 strains expressing B.1.1.28 or B.1.351 spike proteins, reduced (3 to 8-fold) neutralization of the variant viruses were also observed although the titers remained >1,000. At six weeks post IN immunization of ChAd-SARS-CoV-2-S, K18-hACE2 mice were fully protected mice against weight loss and infection in the upper and lower respiratory tracts and brain by WA1/2020, Wash-B.1.351, and Wash-B.1.1.28. Remarkably, in a separate cohort of K18-hACE2 mice challenged nine months after single IN immunization, animals were fully protected against Wash-B.1.351 challenge. Although correlates of protection are not fully established for SARS-CoV-2 vaccines, the high levels of cross-neutralizing antibodies against the variant viruses combined with robust virus-specific systemic and mucosal CD8⁺ T cell responses likely contribute to protection. Beyond this, antibody effector functions also might contribute to prevent SARS-CoV-2 infection and disease. Indeed, enhanced Fc effector functions against SARS-CoV-2 variant proteins were observed in serum derived from IN-delivered ChAd-SARS-CoV-2-S including robust induction of ADNP and ADCP responses.

In summary, the present example shows that IN immunization with ChAd-SARS-CoV-2-S induces robust and durable binding IgG and IgA antibody, neutralizing antibody, Fc effector functions, and LLPC responses against SARS-CoV-2. In mice, a single IN immunization with ChAd-SARS-CoV-2-S confers cross-protection against SARS-CoV-2 strains displaying spike proteins corresponding to B.1.351 and B.1.1.28 variants, even nine months after vaccination. Given the efficacy of preclinical evaluation in multiple animal models 30, 31, 32 and the durable protective immunity against variants of concern, IN delivery of ChAd-SARS-CoV-2-S is a promising platform for preventing SARS-CoV-2 infection, and curtailing transmission.

Methods

Viruses and cells: Vero E6 (CRL-1586, American Type Culture Collection (ATCC), Vero-TMPRSS2 57, Vero (CCL-81, ATCC) and HEK293 (CRL-1573, ATCC) cells were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, and 100 U/ml of penicillin-streptomycin. Vero-TMPRSS2 cells also were supplemented with 5 μg/mL of blasticidin.

SARS-CoV-2 strain 2019n-CoV/USA_WA1/2020 (WA1/2020) was obtained from the Centers for Disease Control and Prevention. The virus was passaged once in Vero CCL-81 cells and titrated by focus-forming assay (FFA) on Vero E6 cells. The Wash-B.1.351 and Wash-B.1.1.28 chimeric viruses with variant spike genes were described previously 28, 58. All viruses were passaged once in Vero-TMPRSS2 cells and subjected to next-generation sequencing to confirm the introduction and stability of substitutions. All virus experiments were performed in an approved Biosafety level 3 (BSL-3) facility.

Mouse experiments: Animal studies were carried out in accordance with the recommendations in the Guide for the Care and Use of Laboratory Animals of the National Institutes of Health. The protocols were approved by the Institutional Animal Care and Use Committee at the Washington University School of Medicine (Assurance number A3381-01). Virus inoculations were performed under anesthesia that was induced and maintained with ketamine hydrochloride and xylazine, and all efforts were made to minimize animal suffering.

Female BALB/c (catalog 000651) and K18-hACE2 C57BL/6 (catalog 034860) mice were purchased from The Jackson Laboratory. Four to five-week-old animals were immunized with 10¹⁰ vp of ChAdV-control or 10⁸, 10⁹, or 10¹⁰ vp of ChAd-SARS-CoV-2-S in 50 μl PBS via IM (hind leg) or IN injection. Vaccinated BALB/c mice (10 to 11-week-old) were given a single intraperitoneal injection of 2 mg of anti-Ifnar1 mAb (MAR1-5A3 59 (Leinco) one day before IN administration of 10⁸ PFU of Hu-Ad5-hACE2 37. Five days after Hu-Ad5-hACE2 transduction, mice were inoculated with 4×10⁵ FFU of WA1/2020 SARS-CoV-2 by the IN route. K18-hACE2 mice were challenged on indicated days after immunization with 10⁴ FFU of SARS-CoV-2 (WA1/2020, Wash-B.1.351, or Wash-B.1.1.28) via IN route. Animals were euthanized at 6 dpi, and tissues were harvested for virological analysis.

Chimpanzee and human adenovirus vectors: The ChAd-SARS-CoV-2 and ChAd-Control vaccine vectors were derived from simian Ad36 backbones 60, and the constructing and validation has been described herein. The rescued replication-incompetent ChAd-SARS-CoV-2-S and ChAd-Control vectors were scaled up in HEK293 cells and purified by CsCl density-gradient ultracentrifugation. Viral particle concentration in each vector preparation was determined by spectrophotometry at 260 nm. The Hu-AdV5-hACE2 vector also was described above and produced in HEK293 cells. The viral titer was determined by plaque assay in HEK293 cells.

SARS-CoV-2 neutralization assays: Heat-inactivated serum samples were diluted serially and incubated with 10² FFU of different SARS-CoV-2 strains for 1 h at 37° C. The virus-serum mixtures were added to Vero cell monolayers in 96-well plates and incubated for 1 h at 37° C. Subsequently, cells were overlaid with 1% (w/v) methylcellulose in MEM supplemented with 2% FBS. Plates were incubated for 30 h before fixation using 4% PFA in PBS for 1 h at room temperature. Cells were washed then sequentially incubated with an oligoclonal pool of SARS2-2, SARS2-11, SARS2-16, SARS2-31, SARS2-38, SARS2-57, and SARS2-71 62 anti-S antibodies and HRP-conjugated goat anti-mouse IgG (Sigma, 12-349) in PBS supplemented with 0.1% saponin and 0.1% bovine serum albumin. TrueBlue peroxidase substrate (KPL) was used to develop the plates before counting the foci on a BioSpot analyzer (Cellular Technology Limited).

Protein expression and purification: The cloning and production of purified S and RBD proteins corresponding to the WA1/2020 SARS-CoV-2 strain have been described previously. Briefly, prefusion-stabilized S 64 and RBD were cloned into a pCAGGS mammalian expression vector with a hexahistidine tag and transiently transfected into Expi293F cells. Proteins were purified by cobalt-charged resin chromatography (G-Biosciences).

ELISA: Purified antigens (S or RBD) were coated onto 96-well Maxisorp clear plates at 2 μg/mL in 50 mM Na₂CO₃ pH 9.6 (70 μL) overnight at 4° C. Coating buffers were aspirated, and wells were blocked with 200 μL of 1×PBS+0.05% Tween-20+1% BSA+0.02% NaN₃ (Blocking buffer, PBSTBA) overnight at 4° C. Heat-inactivated serum samples were diluted in PBSTBA in a separate 96-well polypropylene plate. The plates then were washed thrice with 1×PBS+0.05% Tween-20 (PBST), followed by addition of 50 μL of respective serum dilutions. Sera were incubated in the blocked ELISA plates for at least 1 h at room temperature. The ELISA plates were again washed thrice in PBST, followed by addition of 50 μL of 1:1,000 anti-mouse IgG-HRP (Southern Biotech Cat. #1030-05) in PBST or 1:1000 of anti-mouse IgA-HRP in PBSTBA (SouthernBiotech). Plates were incubated at room temperature for 1 h, washed thrice in PBST, and then 100 μL of 1-Step Ultra TMB-ELISA was added (ThermoFisher Cat. #34028). Following a 10 to 12-min incubation, reactions were stopped with 50 μL of 2 M sulfuric acid. Optical density (450 nm) measurements were determined using a microplate reader (Bio-Rad).

EL/SPOT assay: To quantitate S-specific plasma cells in the bone marrow, femurs and tibias were crushed using a mortar and pestle in RPMI 1640, filtered through a 100 μm strainer and subjected to ACK lysis. CD138⁺ cells were enriched by positive selection and magnetic beads according to the manufacturer's instructions (EasySep Mouse CD138 Positive Selection, STEMCELL). The enriched CD138⁺ cells were incubated overnight in RPMI 1640 supplemented with 10% FBS in MultiScreen-HA Filter Plates (Millipore) pre-coated with SARS-CoV-2 S protein. Foci were developed using TruBlue substrate (KPL) following sequential incubation with anti-mouse IgG-biotin or anti-mouse IgA-biotin and streptavidin-HRP. Plates were imaged using a BioSpot instrument, and foci enumerated manually.

Measurement of viral burden: SARS-CoV-2 infected mice were euthanized using a ketamine and xylazine cocktail, and organs were collected. Tissues were weighed and homogenized with beads using a MagNA Lyser (Roche) in 1 ml of Dulbecco's Modified Eagle's Medium (DMEM) containing 2% fetal bovine serum (FBS). RNA was extracted from clarified tissue homogenates using MagMax mirVana Total RNA isolation kit (Thermo Scientific) and the KingFisher Flex extraction system (Thermo Scientific). SARS-CoV-2 RNA levels were measured by one-step quantitative reverse transcriptase PCR (qRT-PCR) TaqMan assay as described previously 37. SARS-CoV-2 nucleocapsid (N) specific primers and probe sets were used as described above. Viral RNA was expressed as (N) gene copy numbers per milligram on a log 10 scale.

Luminex analysis: Luminex analysis was conducted as described previously. Briefly, proteins (Spike: D614G, E484K, N501 Δ69-70, K417N, B.1.1.7, B.1.351; Receptor Binding Domain (RBD) (ImmuneTech): WT, E484K, B.1.1.7, B.1.351, B.1.128) were carboxy-coupled to magnetic Luminex microplex carboxylated beads (Luminex Corporation) using NHS-ester linkages with Sulfo-NHS and EDC (Thermo Fisher) and then incubated with serum (IgG1, FcγRIIb, FcγRIII 1:3000; IgG2a, G2b, G3, A, FcγRIV 1:1000, IgM 1:500) for 2 h at 37° C. Isotype analysis was performed by incubating the immune complexes with secondary goat anti-mouse-PE antibody (IgG1 1070-09, IgG2a 1080-09S, IgG2b 1090-09S, IgG3 1100-09, IgM 1020-09, IgA 1040-09 Southern Biotech) for each isotype. FcγR binding was quantified by incubating immune complexes with biotinylated FcγRs (FcγRIIB, FcγRIII, and FcγRIV, courtesy of Duke Protein Production Facility) conjugated to Steptavidin-PE (Prozyme). Flow cytometry was performed with an IQue (Intellicyt) and analysis was performed on IntelliCyt ForeCyt (v8.1).

Antibody-dependent neutrophil or cellular phagocytosis: Antibody-dependent neutrophil phagocytosis (ADNP) and cellular phagocytosis (ADCP) assays were conducted as described previously. Briefly, spike protein was carboxy coupled to blue, yellow-green, or red FluoSphere™ Carboxylate-modified microsphere, 0.2 μm (ThermoFisher) using NHS-ester linkages with Sulfo-NHS and EDC (Thermo Fisher). Spike-coated beads were incubated with diluted serum (1:150 ADNP, 1:100 ADCP) for 2 hours at 37° C. For the ADNP assay, bone marrow cells were collected from BALB/c mice, and red blood cells were subjected to ACK lysis. The remaining cells were washed with PBS, and aliquoted into 96-well plates (5×10⁴ cells per well). The bead-antibody complexes were added to cells and incubated for 1 h at 37° C. After washing, cells were stained with the following antibodies: CD11b APC (BioLegend 101212), CD11c A700 (BioLegend 117320), Ly6G Pacific Blue (127628), Ly6C BV605 (BioLegend 128036), Fcblock (BD Bioscience 553142) and CD3 PE/Cy7 (BioLegend 100320). Cells were fixed with 4% PFA, processed on an BD LSRFortessa (BD Biosciences). Neutrophils were defined as CD3⁻, CD11b⁺, Ly6G⁺. The neutrophil phagocytosis score was calculated as (% FITC+)×(geometic mean fluorescent intensity of FITC)/10000. For the ADCP assay, J774A.1 (ATCC TIB-67) murine monocytic cells were incubated with the Spike-coated bead-antibody complexes for 1 h at 37° C. Cells were washed in 5 mM EDTA PBS, fixed with 4% PFA, and analyzed on an BD LSRFortessa (BD Biosciences). The cellular phagocytosis score was calculated as (% FITC+)×(geometic mean fluorescent intensity of FITC)/10000.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

What is claimed is:

1. An adenoviral vector comprising the genome of a non-human adenovirus, wherein the genome of the adenovirus has been modified such that the vector lacks the native E1 and optionally, the E3 or E3B locus, and comprises a transgene operably linked to expression control sequences which direct transcription, translation, and/or expression in a host cell, wherein the transgene encodes a SARS-CoV-2 spike (S) protein or an immunogenic portion, variant, mutant, or fragment thereof.

2. The adenoviral vector of claim 1, wherein the non-human adenovirus is a simian adenovirus (SAdV).

3. The adenoviral vector of claim 2, wherein the SAdV is simian adenovirus 36.

4. The adenoviral vector of claim 1, wherein the adenoviral genome has the nucleic acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity with SEQ ID NO: 4.

5. The adenoviral vector of claim 1, wherein the adenoviral genome has the nucleic acid sequence of SEQ ID NO: 4.

6. The adenoviral vector of any one of claims 1-5, wherein the transgene encodes a stabilized prefusion form of the SARS-CoV-2 spike (S) protein.

7. The adenoviral vector of any one of claims 1-5, wherein the transgene encodes a coronavirus spike protein (S) protein having an amino acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 3.

8. The adenoviral vector of any one of claims 1-5, wherein the transgene encodes a coronavirus spike protein (S) protein having an amino acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 3 having K986P and V987P mutations.

9. The adenoviral vector of claim 1, wherein the transgene is a coronavirus S protein from a WA1/2020, B.1.1.7, B.1.351, B.1.1.28, P.1, B.1.427, B.1.526, B.1.526.1, B.1.525, P.2, B.1.617, B.1.617.1, B.1.617.2, B.1.617.3, B.1.429, or B.1.429 variant.

10. The adenoviral vector of claim 1, wherein the adenoviral vector has a nucleic acid sequence with at least 80%, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% sequence identity to SEQ ID NO: 2, SEQ ID NO: 5 or SEQ ID NO: 6.

11. The adenoviral vector of claim 1, wherein the adenoviral vector has a nucleic acid sequence of SEQ ID NO: 2, SEQ ID NO: 5 or SEQ ID NO: 6.

12. A pharmaceutical composition, comprising the adenoviral vector of any one of claims 1-11 and a pharmaceutically acceptable carrier, diluent, excipient, or adjuvant.

13. The pharmaceutical composition of claim 12, wherein the composition is formulated for intranasal administration.

14. An immunogenic composition comprising the adenoviral vector according to any one of the preceding claims and optionally one or more additional active ingredients, a pharmaceutically acceptable carrier, diluent, excipient, or adjuvant.

15. A host cell transduced with the composition or adenoviral vector of any one of the preceding claims.

16. A packaging cell line producing the composition or viral vector of any one of the preceding claims.

17. The packaging cell line of claim 16, wherein the cell comprises the complement of any adenoviral genes functionally deleted in the viral vector of any one of the preceding claims.

18. A kit comprising: (i) the host cell, the cell line, the composition, or the adenoviral vector according to any one of claim 1-17 or an immunogenic composition thereof, and (ii) instructions for use.

19. A coronavirus vaccine comprising the composition or adenovirus vector of any one of claims 1-14.

20. A composition comprising the sera of a subject previously administered the composition of any one of claims 1-14.

21. A method of treating a second subject having a coronavirus infection comprising administering to the subject an immunogenically effective amount of the composition comprising the sera according to claim 20.

22. A method for inducing an immune response against coronavirus in a subject, comprising administering to the subject an immunogenically effective amount of a composition according to any one of claims 1-14 or 19-20.

23. A method of treating or preventing a coronavirus infection in a subject, comprising administering to the subject an immunogenically effective amount of a composition according to any one of claims 1-14 or 19-20.

24. A method for protecting a subject from coronavirus infection, comprising administering to the subject an immunogenically effective amount of a composition according to any one of claims 1-14 or 19-20.

25. The method of any one of claims 21-24, wherein the composition is administered intranasally.

26. The method of any one of claims 21-25, wherein the immunogenically effective amount of a composition e protects against SARS-CoV-2 infection in the nasal passages, upper airways, lung tissues, and all other sites of dissemination and/or prevents upper respiratory tract infection and nasal virus shedding.

27. The method of any one of claims 21-24, wherein the immunogenically effective amount of a composition is administered intramuscularly.

28. The method of any one of claims 21-27, wherein the coronavirus is a SARS-CoV-2 virus, SARS-CoV-2 variant comprising the D614G mutation, or a SARS-CoV-2 mutant.

29. The method of any one of claims 21-28, wherein the subject is a human.

30. The method of any one of claims 21-29, wherein the subject has been exposed to a coronavirus.

31. The method of any one of claims 21-30, wherein the subject does not have, but is at risk of developing a coronavirus infection.

32. The method of any one of claims 21-29, wherein the subject is traveling to a region where the coronavirus is prevalent.

33. The method of any one of claims 21-32, wherein administration of the composition results in an antigen-specific immune response.

34. The method of any one of claims 21-33, wherein the subject has, is suspected of having, or is at risk for developing a coronavirus infection.

35. The method of any one of claims 21-34, wherein the method comprises delivering a transgene into a host cell of the subject.

36. A method of making a recombinant adenovirus comprising: transfecting a cell with the viral vector according to any one of claims 1-11; culturing the cell under conditions such that the cell produces the recombinant adenovirus; and collecting the recombinant adenovirus.

37. The method of claim 36, wherein the cells are HEK cells, Vero, or PER cells.


SEQUENCE LISTING (9 sequences)

SEQ ID NO: 1 - DNA, 6602 bp, Artificial Sequence
  Synthesized
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatccactcccaggtccaactgcagccggtaccgccaccatgtttgtctttctcgtgctcctccctctcgtctcatcccagtgcgtgaacctgactactcggacccagctccctcccgcttacaccaactccttcacaaggggcgtgtactaccccgacaaggtgtttagaagctccgtgctgcactctacacaggatctgtttctgcctttctttagcaacgtgacctggttccacgctatccacgtgtctggcaccaacggaacaaagaggttcgacaacccagtgctgccctttaacgatggcgtgtacttcgcctctaccgagaagagcaacatcatcagaggctggatctttggaaccacactggactccaagacacagtctctgctgatcgtgaacaacgccaccaacgtggtcatcaaggtgtgcgagttccagttttgtaacgatccattcctgggcgtgtactaccacaagaacaacaagagctggatggagtccgagtttagagtgtactctagcgccaacaactgcacatttgagtacgtgtcccagcccttcctgatggacctggagggcaagcagggaaacttcaagaacctgcgggagttcgtgtttaagaacatcgatggctactttaagatctactctaagcacaccccaatcaacctggtgcgcgacctgccacagggcttcagcgccctggagccactggtggatctgcccatcggaatcaacatcaccaggtttcagacactgctggccctgcacagaagctacctgacaccaggcgactcctctagcggatggaccgctggagctgctgcttactacgtgggctacctgcagccccggaccttcctgctgaagtacaacgagaacggaaccatcacagacgctgtggattgcgccctggatcccctgagcgagacaaagtgtacactgaagtcctttaccgtggagaagggcatctaccagacatccaacttccgggtgcagcctaccgagtctatcgtgcgctttcccaacatcacaaacctgtgcccttttggagaggtgttcaacgctacccgcttcgccagcgtgtacgcttggaaccggaagcgcatctctaactgcgtggccgactacagcgtgctgtacaactccgcttctttcagcacctttaagtgctacggcgtgtcccctacaaagctgaacgacctgtgctttaccaacgtgtacgccgattctttcgtgatcaggggagacgaggtgcgccagatcgctcccggccagacaggaaagatcgctgactacaactacaagctgcctgacgatttcaccggctgcgtgatcgcctggaacagcaacaacctggattccaaagtgggcggaaactacaactacctgtacaggctgtttagaaagagcaacctgaagccattcgagcgggacatctctacagagatctaccaggctggaagcaccccatgcaacggagtggagggcttcaactgttacttccctctgcagtcctacggattccagccaacaaacggcgtgggataccagccctaccgcgtggtggtgctgagctttgagctgctgcacgctcctgctacagtgtgcggaccaaagaagtccaccaacctggtgaagaacaagtgcgtgaacttcaactttaacggactgaccggcacaggagtgctgaccgagtccaacaagaagttcctgccttttcagcagttcggccgggacatcgccgataccacagacgctgtgcgcgaccctcagaccctggagatcctggacatcacaccatgctctttcggcggagtgagcgtgatcacaccaggaaccaacacaagcaaccaggtggccgtgctgtaccaggacgtgaactgtaccgaggtgcccgtggctatccacgccgatcagctgacccctacatggagggtgtacagcaccggctccaacgtgttccagacaagagccggctgtctgatcggagctgagcacgtgaacaactcctacgagtgcgacatccctatcggcgccggaatctgtgcttcttaccagacccagacaaactctccaaggagagccaggagcgtggcttcccagtctatcatcgcctacaccatgtccctgggcgccgagaactctgtggcttactctaacaacagcatcgctatccctaccaacttcacaatctctgtgaccacagagatcctgccagtgtccatgaccaagacatctgtggactgcacaatgtacatctgtggagattctaccgagtgcagcaacctgctgctgcagtacggcagcttttgtacccagctgaacagagccctgacaggaatcgctgtggagcaggacaagaacacacaggaggtgttcgcccaggtgaagcagatctacaagaccccccctatcaaggactttggcggattcaacttttcccagatcctgcccgatccttccaagccatctaagaggagctttatcgaggacctgctgttcaacaaggtgaccctggctgatgccggcttcatcaagcagtacggcgattgcctgggagacatcgctgccagggacctgatctgtgcccagaagtttaacggactgaccgtgctgccacccctgctgacagatgagatgatcgctcagtacacaagcgccctgctggctggaaccatcacatccggatggaccttcggcgctggagctgccctgcagatcccctttgccatgcagatggcttaccggttcaacggcatcggagtgacccagaacgtgctgtacgagaaccagaagctgatcgccaaccagtttaactccgctatcggcaagatccaggacagcctgtcctctacagcttccgccctgggaaagctgcaggatgtggtgaaccagaacgctcaggccctgaacaccctggtgaagcagctgagctccaacttcggcgccatctctagcgtgctgaacgacatcctgagcaggctggacaaggtggaggctgaggtgcagatcgacaggctgatcacaggaagactgcagtctctgcagacctacgtgacacagcagctgatcagggctgctgagatcagggctagcgccaacctggctgccaccaagatgtccgagtgcgtgctgggccagtctaagagagtggacttttgtggcaagggataccacctgatgtccttcccccagtctgcccctcacggagtggtgtttctgcacgtgacctacgtgccagctcaggagaagaacttcaccacagctcccgccatctgccacgatggcaaggcccactttcctcgggagggcgtgttcgtgtccaacggaacccactggtttgtgacacagcgcaacttctacgagccacagatcatcaccacagacaacacattcgtgtctggcaactgtgacgtggtcatcggaatcgtgaacaacaccgtgtacgatcctctgcagccagagctggacagctttaaggaggagctggataagtacttcaagaaccacacctcccccgacgtggatctgggcgacatcagcggaatcaacgcctccgtggtgaacatccagaaggagatcgacaggctgaacgaggtggctaagaacctgaacgagagcctgatcgatctgcaggagctgggcaagtacgagcagtacatcaagtggccttggtacatctggctgggcttcatcgccggactgatcgctatcgtgatggtgaccatcatgctgtgctgtatgacatcctgctgttcttgcctgaagggctgctgtagctgtggatcctgctgtaaattcgatgaggacgattccgagcctgtgctgaagggcgtgaaactccattacacctgaatcgattgcggccgctctcgagtctagctgatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 40403 bp, Artificial Sequence
  Synthesized
taaccccatcatcaataatatacctcaaacttttggtgcgcgttaatatgcaaatgagctgtttgaatttggggatgcggggctgtgattggctgtgggagcggcgaccgttaggggcggggcgggtgacgttttgatgacgtgtttgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacatcatttccccgaaaagtgccacctgacgtaactataacggtcctaaggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcaatcgattcaggtgtaatggagtttcacgcccttcagcacaggctcggaatcgtcctcatcgaatttacagcaggatccacagctacagcagcccttcaggcaagaacagcaggatgtcatacagcacagcatgatggtcaccatcacgatagcgatcagtccggcgatgaagcccagccagatgtaccaaggccacttgatgtactgctcgtacttgcccagctcctgcagatcgatcaggctctcgttcaggttcttagccacctcgttcagcctgtcgatctccttctggatgttcaccacggaggcgttgattccgctgatgtcgcccagatccacgtcgggggaggtgtggttcttgaagtacttatccagctcctccttaaagctgtccagctctggctgcagaggatcgtacacggtgttgttcacgattccgatgaccacgtcacagttgccagacacgaatgtgttgtctgtggtgatgatctgtggctcgtagaagttgcgctgtgtcacaaaccagtgggttccgttggacacgaacacgccctcccgaggaaagtgggccttgccatcgtggcagatggcgggagctgtggtgaagttcttctcctgagctggcacgtaggtcacgtgcagaaacaccactccgtgaggggcagactgggggaaggacatcaggtggtatcccttgccacaaaagtccactctcttagactggcccagcacgcactcggacatcttggtggcagccaggttggcgctagccctgatctcagcagccctgatcagctgctgtgtcacgtaggtctgcagagactgcagtcttcctgtgatcagcctgtcgatctgcacctcagcctccaccttgtccagcctgctcaggatgtcgttcagcacgctagagatggcgccgaagttggagctcagctgcttcaccagggtgttcagggcctgagcgttctggttcaccacatcctgcagctttcccagggcggaagctgtagaggacaggctgtcctggatcttgccgatagcggagttaaactggttggcgatcagcttctggttctcgtacagcacgttctgggtcactccgatgccgttgaaccggtaagccatctgcatggcaaaggggatctgcagggcagctccagcgccgaaggtccatccggatgtgatggttccagccagcagggcgcttgtgtactgagcgatcatctcatctgtcagcaggggtggcagcacggtcagtccgttaaacttctgggcacagatcaggtccctggcagcgatgtctcccaggcaatcgccgtactgcttgatgaagccggcatcagccagggtcaccttgttgaacagcaggtcctcgataaagctcctcttagatggcttggaaggatcgggcaggatctgggaaaagttgaatccgccaaagtccttgatagggggggtcttgtagatctgcttcacctgggcgaacacctcctgtgtgttcttgtcctgctccacagcgattcctgtcagggctctgttcagctgggtacaaaagctgccgtactgcagcagcaggttgctgcactcggtagaatctccacagatgtacattgtgcagtccacagatgtcttggtcatggacactggcaggatctctgtggtcacagagattgtgaagttggtagggatagcgatgctgttgttagagtaagccacagagttctcggcgcccagggacatggtgtaggcgatgatagactgggaagccacgctcctggctctccttggagagtttgtctgggtctggtaagaagcacagattccggcgccgatagggatgtcgcactcgtaggagttgttcacgtgctcagctccgatcagacagccggctcttgtctggaacacgttggagccggtgctgtacaccctccatgtaggggtcagctgatcggcgtggatagccacgggcacctcggtacagttcacgtcctggtacagcacggccacctggttgcttgtgttggttcctggtgtgatcacgctcactccgccgaaagagcatggtgtgatgtccaggatctccagggtctgagggtcgcgcacagcgtctgtggtatcggcgatgtcccggccgaactgctgaaaaggcaggaacttcttgttggactcggtcagcactcctgtgccggtcagtccgttaaagttgaagttcacgcacttgttcttcaccaggttggtggacttctttggtccgcacactgtagcaggagcgtgcagcagctcaaagctcagcaccaccacgcggtagggctggtatcccacgccgtttgttggctggaatccgtaggactgcagagggaagtaacagttgaagccctccactccgttgcatggggtgcttccagcctggtagatctctgtagagatgtcccgctcgaatggcttcaggttgctctttctaaacagcctgtacaggtagttgtagtttccgcccactttggaatccaggttgttgctgttccaggcgatcacgcagccggtgaaatcgtcaggcagcttgtagttgtagtcagcgatctttcctgtctggccgggagcgatctggcgcacctcgtctcccctgatcacgaaagaatcggcgtacacgttggtaaagcacaggtcgttcagctttgtaggggacacgccgtagcacttaaaggtgctgaaagaagcggagttgtacagcacgctgtagtcggccacgcagttagagatgcgcttccggttccaagcgtacacgctggcgaagcgggtagcgttgaacacctctccaaaagggcacaggtttgtgatgttgggaaagcgcacgatagactcggtaggctgcacccggaagttggatgtctggtagatgcccttctccacggtaaaggacttcagtgtacactttgtctcgctcaggggatccagggcgcaatccacagcgtctgtgatggttccgttctcgttgtacttcagcaggaaggtccggggctgcaggtagcccacgtagtaagcagcagctccagcggtccatccgctagaggagtcgcctggtgtcaggtagcttctgtgcagggccagcagtgtctgaaacctggtgatgttgattccgatgggcagatccaccagtggctccagggcgctgaagccctgtggcaggtcgcgcaccaggttgattggggtgtgcttagagtagatcttaaagtagccatcgatgttcttaaacacgaactcccgcaggttcttgaagtttccctgcttgccctccaggtccatcaggaagggctgggacacgtactcaaatgtgcagttgttggcgctagagtacactctaaactcggactccatccagctcttgttgttcttgtggtagtacacgcccaggaatggatcgttacaaaactggaactcgcacaccttgatgaccacgttggtggcgttgttcacgatcagcagagactgtgtcttggagtccagtgtggttccaaagatccagcctctgatgatgttgctcttctcggtagaggcgaagtacacgccatcgttaaagggcagcactgggttgtcgaacctctttgttccgttggtgccagacacgtggatagcgtggaaccaggtcacgttgctaaagaaaggcagaaacagatcctgtgtagagtgcagcacggagcttctaaacaccttgtcggggtagtacacgccccttgtgaaggagttggtgtaagcgggagggagctgggtccgagtagtcaggttcacgcactgggatgagacgagagggaggagcacgagaaagacaaacatggtggcggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagaggcctgaccatctggtgctggcctgcaccagggccgagtttgggtctagcttaagtttgatccgatctttttccctctgccaaaaattatggggacatcatgaagccccttgagcatctgacttctggctaataaaggaaatttattttcattgcaatagtgtgttggaattttttgtgtctctcactcggaagcgatctgaattcatctatgtcgggtgcggagaaagaggtaatgaaatggcattatgggtattatgggtctgcattaatgaatcggccagattatgctggccaccgtgcatgtggcctcgcacccccgcaagacatggcccgagttcgagcacaacgtcatgacccgatgcaacgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgggagtctgcatgatgggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcaaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggctctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgcccgcccaggttttccatgcactcatccatgatgatggcgatgggcccgtgggcggcggcctgggcaaagacgtttcgggggtcggacacatcatagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtgggaccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagttcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcatgccccagggcatggggtgcgtgagcgcagaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgcgccgaggttggagcgctgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttgatgttgtggcccacgatgtagagttccacgaaccgtgggcggcccttgacgtggggcagcttcttgagctcctcgtaggtgagctcgtcagggtcgctgagcccgtgctgctcgagggcccagtcggcgagatggtggttggcgcggaggaaggaagtccagagatccacggccagggcggtttgcagacggtcccggtactgacggaactgctggccgacggccattttttcgggggtgacgcagtagaaggtgcgggggtccccgtgccagcgatcccatttgagctggagggcgagatcgagggcgagctcgacgaggcggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggagtgaccaccgtcccccgtttcttcttgggcgctgcttccatgccggtcagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggccccgggaacctcttcttccatctcctcttcttcctcttccactaacatctcttctacttcctcctcaggcggtggcgggggagggggcctgcgtcgccggcggcgcacgggcagacggtcgatgaagcgctcgatggtctcgccgcgccggcgtcgcatggtctcggtgacggcccgcccgtcctcgcggggccgcagcgtgaagacgccgccgcgcatttccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctggcgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccagatccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggccgggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgtttttgcaactttttttcggaggccggaaatgaagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagacccctagccagccgacttctccagttacggagcgagcccctcttttgttttttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctaaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgagcggtgagcgccagcaggcggcgcgagctgagcgaccaggagctgatgcacagcctgcagcgggccctgaccggggccgggaccgagggggagagctactttgacatgggcgcggacctgcactggcaacccagccgccgggccttggaggcggcggcaggaccctacgtagaagaggtggacgatgaggtggacgagggcgagtacctggaagactgatggcgcgaccgtatttttgctagatgcaacaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagaccaacctggacaggatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagtatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacgcgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctaaacccccactggctaccgccgccggggttctacacgggcgagtacgacatgcccgaccccaatgacgggttcctgtgggacgatgtggacagcagcgtgttttccccccgaccgggtgctaacgagcgccccttgtggaagaaggaaggcagcgaccgacgcccgtcctcggcgctgtccggccgcgagggtgctgccgcggcggtgcccgaggccgccagtccttttcctagcttgcccttctcgctgaacagtattcgcagcagcgagctgggcaggatcacgcgcccgcgcttgctcggcgaggaggagtacttgaatgactcgctgttgagacccgagcgggagaagaacttccccaataacgggatagagagcctggtggacaagatgagccgctggaagacgtacgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtggtggtaacccgttcgctcacctgcgcccccgcatcgggcgcatgatgtaagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgaattcatgtacagcaacaagttcaaggcgcgggtcatggtctcccgcaagacccccaatggggtcaaagtagatgaaaattatgatggtagtcaggatgagctgaaatacgagtgggtggagtttgagctgcccgaaggcaacttctcggtgaccatgaccatcgacctgatgaacaacgccatcatcgacaattacttggcggtggggcggcagaacggggtcctggaaagcgacatcggcgtgaagttcgacactaggaacttcaggctgggctgggaccccgtgaccgagctggtcatgcccggggtgtacaccaacgaggccttccatcccgatgttgtcttgctgcccggctgcggggtggactttaccgagagccgcctcagcaacctgctgggcattcgcaagaggcagcccttccaggagggattccagatcatgtacgaggatctggaggggggcaacatccccgcgctcctggatgtcgaggcctatgaggaaagcaaggaaaaagctgaagccgaggcgactgcagccgtggctaccgccgcgaccaccaatgcagatgcaactaccaccagaggcgatacattcgccactgtggcggaggaagcagccgccctagcggtcgccgatgatagtgaaagtaagatagttatcaagccagtaaaagtggatagcaagaacagaagctacaacgtgctgccggacgaggtaaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcagccagacgcgcggcctccggcagcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggatggactggtggagtttgtgcgcgagttcgccccccggcggcgcgtgcagtggcgcgggcggaaagtgaagccggtgctgcggccaggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgcccgcggcttccaccgccacccgccgaagacgcaagtacggcgcggccagcctgctgatgcccaactacgcgctgcatccttccatcatccccacgccgggctaccgcggcacgcgcttctaccgcggctacaccagccgccgccgcaagaccaccacccgccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccccagtttcaaaccctactccggcaccgcctacaacagcctggctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgataccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtgggcaggctaacataaaagattctgaaactgccagcactactcctaactatgatatagatttggcattctttgacagcaaaaatattgccgctaactatgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctgaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtgggctttacagatacttaccagggtgttaaagttaagacagatacagccgctgctggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccggccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgttgtccgagtgcaccagccccaccgcggcgtcatcgaggccgtctacctgcgcacgcccttctcggccggcaacgccaccacctaagccccgctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgtctactttcgctcccaccgcgcgcgcatcgagaaggccaccgccttcgaccgcatgaatcaagacatgtaaactgtgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccatatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcagccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgagacgatcggcaggctcagggggttcttcaccgttgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcgtgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgcagccgacgaaaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctccgaggacgagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgttacttgctgcgcctggccaactacctggcctaccactcggacgtgatcgaggacgtcagcggcgagggtctgctcgaatgccactgccgctgcaacctctgcacgccgcaccgctccctggcctgcaacccccagctgctgagcgaaacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaggaagacgaggtggaggaggaggcagaggaagaagcagccgccgccagaccgtcgtcctcggcggaggaggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagctagaaaatccacagcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacatacccgtctgaccctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgccgctccccgctactaacaaccaaactacccaccaacgccgccgtcgcgacctttctgaatctaatactaccacccacaccggaggtgagctccgaggtcgaccaacctctgggatttactacggcccctgggaggtggtagggttaatagcgctaggcctagttgcgggtgggcttttggctctctgctacctatacctcccttgctgttcgtacttagtggtgctgtgttgctggtttaagaaatggggaagatcaccctagtgagctgcggtgtgctggtggcggtggtggtgctttcgattgtgggactgggcggcgcggctgtagtgaaggaggagaaggccgatccctgcttgcatttcaatcccgacaaatgccagctgagttttcagcccgatggcaatcggtgcacggtgctgattaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcacacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattaacatgttcatcgctattcgccccagaaataatgccgaaaaagagaaacagccataacacgtttttttcacacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcattttggacatggaattttatcaagtttctgtgtctgaacccaccacgcctagaatgaccacaaccacaaaaactacacctgttaccactatacagctcactaccaatggctttcttgccatgcttcaagtggctgaaaatagcaccagcattcaacccaccccacccagtgaggaaattcccagatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggttgctcgtcagcggtattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgtttttagtgctgggtttttgaaaaatcttaccatttatgaaggtgagaatgccactctagtgggcatcagtggtcaaaatgtcagctggctaaaataccatctagatgggtggaaagacatttgcgattggaatgtcactgtgtatacatgtaatggagttaacgcgatcgctcacccccttatccagtgaaataaagatcatattgatgatgatttaaataaaaaaataatcatttgatttgaaataaagatacaatcatattgatgatttgagtttaacaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcatcccttttacactaaagatggaaaattatccttacaagtttctccaccattaaatatactgagaacaagcattctaaacacactagctttaggttttggatcaggtttaggactccgtggctctgccttagcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaacggagatgtttcaaaacctatgcttctcactataaccctcaatggtactgatgacagcaacagtacatattcaatgtcattttcatacacctggactaatggaagctatgttggagcaacatttggggctaactcttataccttctcctacattgcccaagaatgaacactgtatcccaccctacattgcccaacccttcccaccccactctgtctatggaaaaaactctgaaacacaaaataaaataaagttcaagtgttttattgattcaacagttttacaggattcgagcagttatttttcctccaccctcccaagacatggaatacaccaccctctccccccgcacagccttgaacatttgaaagccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcaaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgtttaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagggaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtcccggcgcttggaacgctcggtgttgaagttgtaaaacagccactctcttagaccgtgcagcaaatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcaaaagcgggagggttctctaattcctcaatcatcatgttacactcctgcaccatccctagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtacgtgcaaggggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcggccgaggaagcaataaggcactacaatgctcagtctcaagtccagcaaagcgatgccatgcggatgaagcacaaaattctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaagactgagctctaacctgtccacccgctctctgctcaatatatagcccagatctacactgacgtaaaggccaaagtctaaaaatacccgccaaataatcacacacgcccagcacacgcccagaaaccggtgacacactcagaaaaatacgcgcacttcctcaaacgcccaaactgccgtcatttccgggttcccacgctacgtcatcagaattcgactttcaaattccgtcgaccgttaaaaatgtcacccgccccgcccctaacggtcgccgctcccacagccaatcacagccccgcatccccaaattcaaacagctcatttgcatattaacgcgcaccaaaagtttgaggtatattattgatgatgatcttaattaagaattaattcgatcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatggctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataatttaat

SEQ ID NO: 3 - protein, 1273 aa, Artificial Sequence
  Synthesized
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - DNA, 36556 bp, Artificial Sequence
  Synthesized
  misc_feature (18533)..(18535): n is a, c, g, or t
  misc_feature (18595)..(18595): n is a, c, g, or t
  misc_feature (18620)..(18620): n is a, c, g, or t
  misc_feature (18668)..(18668): n is a, c, g, or t
  misc_feature (18688)..(18690): n is a, c, g, or t
atcatcaaataatatacctcaaacttttggtgcgcgttaatatgcaaatgagctgtttgaatttggggatgcggggctgtgattggctgtgggagcggcgaccgttaggggcggggcgggtgacgttttgatgacgtgtttgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtaggcgtcagctgatcgccagggtatttaaacctgcgctctctagtcaagaggccactcttgagtgccagcgagtagagttttctcatccgcgccgcgagtcagatctacactttgaaagatgaggcacctgagagacctgcccggtaatgttttcctggctactgggaacgagattctggaactggtggtggacgccatgatgggtgacgaccctcctgagccccataccccatttgaggcgccttcgctgtacgatttgtatgatctggaggtggatgtgcccgagaacgaccccaacgaggaggcggtgaatgatttgtttagcgatgccgcgctgctggctgccgagcaggctaatacggactctggctcagacagcgattcctctctccataccccgagacccggcagaggtgagaaaaagatccccgagcttaaaggggaagagcttgacctgcgctgctatgaggaatgcttgcctccgagcgatgatgaggaggacgaggaggcgattcgagctgcagcgaaccagggagtgaaagcagcgagcgagggctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagttgggaaggcagagggtgactgggtgctgactggtttatttatgtatatgttttttatgtgtaggtcccgtctctgacgcagatgagacccccacttcagagtgcatttcatcacccccagaaattggcgaggaaccgcccgaagatattattcatagaccagttgcagtgagagtcaccgggcggagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtgttttatgactcaggggtgggactgtgggtatataagcaggtgcagacctgtgtggtcagttcagagcaggactcatggagatctggactgtcttggaagactttcaccagactagacagctgctagagaactcatcggagggagtctcttacctgtggagattctgcttcggtgggcctctagctaagctagtctatagggccaaacaggattataaggatcaatttgaggatattttgagagagtgtcctggtatttttgactctctcaacttgggccatcagtctcactttaaccagagtattctgagagcccttgacttttctactcctggcagaactaccgccgcggtagccttttttgcctttatccttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaggaggaccgagaagagaacccgagagccggtctggaccctccggtggcggaggaggaggagtagctgacttgtttcccgagctgcgccgggtgctgactaggtcttccagtggacgggagagggggattaagcgggagaggcatgaggagactagccacagaactgaactgactgtcagtctgatgagccgcaggcgcccagaatcggtgtggtggcatgaggtgcagtcgcaggggatagatgaggtctcagtgatgcatgagaaatattccctagaacaagtcaagacttgttggttggagcccgaggatgattgggaggtagccatcaggaattatgccaagctggctctgaagccagacaagaagtacaagattaccaaactgattaatatcagaaattcctgctacatttcagggaatggggccgaggtggagatcagtacccaggagagggcggccttcagatgttgtatgatgaatatgtacccgggggtggtgggcatggagggagtcacctttatgaacacgaggttcaggggtgatgggtataatggggtggtctttatggccaacaccaagctgacagtgcacggatgctccttctttggcttcaataacatgtgcatcgaggcctggggcagtgtttcagtgaggggatgcagcttttcagccaactggatgggggtcgtgggcagaaccaagagcgtggtttcagtgaagaaatgtctgttcgagaggtgccacctgggggtgatgagcgagggcgaagccaaagtcaaacactgcgcctctaccgagacgggctgctttgtgctgatcaagggcaatgccaaagtcaagcataacatgatctgtggggcctcggatgagcgcggctaccagatgctgacctgcgccggtgggaacagccatatgctggccaccgtgcatgtggcctcgcacccccgcaagacatggcccgagttcgagcacaacgtcatgacccgatgcaacgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgggagtctgcatgatgggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcaaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggctctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgcccgcccaggttttccatgcactcatccatgatgatggcgatgggcccgtgggcggcggcctgggcaaagacgtttcgggggtcggacacatcatagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtgggaccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagttcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcatgccccagggcatggggtgcgtgagcgcagaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgcgccgaggttggagcgctgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttgatgttgtggcccacgatgtagagttccacgaaccgtgggcggcccttgacgtggggcagcttcttgagctcctcgtaggtgagctcgtcagggtcgctgagcccgtgctgctcgagggcccagtcggcgagatggtggttggcgcggaggaaggaagtccagagatccacggccagggcggtttgcagacggtcccggtactgacggaactgctggccgacggccattttttcgggggtgacgcagtagaaggtgcgggggtccccgtgccagcgatcccatttgagctggagggcgagatcgagggcgagctcgacgaggcggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggagtgaccaccgtcccccgtttcttcttgggcgctgcttccatgccggtcagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggccccgggaacctcttcttccatctcctcttcttcctcttccactaacatctcttctacttcctcctcaggcggtggcgggggagggggcctgcgtcgccggcggcgcacgggcagacggtcgatgaagcgctcgatggtctcgccgcgccggcgtcgcatggtctcggtgacggcccgcccgtcctcgcggggccgcagcgtgaagacgccgccgcgcatttccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctggcgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccagatccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggccgggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgtttttgcaactttttttcggaggccggaaatgaagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagacccctagccagccgacttctccagttacggagcgagcccctcttttgttttttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctaaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgagcggtgagcgccagcaggcggcgcgagctgagcgaccaggagctgatgcacagcctgcagcgggccctgaccggggccgggaccgagggggagagctactttgacatgggcgcggacctgcactggcaacccagccgccgggccttggaggcggcggcaggaccctacgtagaagaggtggacgatgaggtggacgagggcgagtacctggaagactgatggcgcgaccgtatttttgctagatgcaacaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagaccaacctggacaggatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagtatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacgcgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctaaacccccactggctaccgccgccggggttctacacgggcgagtacgacatgcccgaccccaatgacgggttcctgtgggacgatgtggacagcagcgtgttttccccccgaccgggtgctaacgagcgccccttgtggaagaaggaaggcagcgaccgacgcccgtcctcggcgctgtccggccgcgagggtgctgccgcggcggtgcccgaggccgccagtccttttcctagcttgcccttctcgctgaacagtattcgcagcagcgagctgggcaggatcacgcgcccgcgcttgctcggcgaggaggagtacttgaatgactcgctgttgagacccgagcgggagaagaacttccccaataacgggatagagagcctggtggacaagatgagccgctggaagacgtacgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtggtggtaacccgttcgctcacctgcgcccccgcatcgggcgcatgatgtaagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgaattcatgtacagcaacaagttcaaggcgcgggtcatggtctcccgcaagacccccaatggggtcaaagtagatgaaaattatgatggtagtcaggatgagctgaaatacgagtgggtggagtttgagctgcccgaaggcaacttctcggtgaccatgaccatcgacctgatgaacaacgccatcatcgacaattacttggcggtggggcggcagaacggggtcctggaaagcgacatcggcgtgaagttcgacactaggaacttcaggctgggctgggaccccgtgaccgagctggtcatgcccggggtgtacaccaacgaggccttccatcccgatgttgtcttgctgcccggctgcggggtggactttaccgagagccgcctcagcaacctgctgggcattcgcaagaggcagcccttccaggagggattccagatcatgtacgaggatctggaggggggcaacatccccgcgctcctggatgtcgaggcctatgaggaaagcaaggaaaaagctgaagccgaggcgactgcagccgtggctaccgccgcgaccaccaatgcagatgcaactaccaccagaggcgatacattcgccactgtggcggaggaagcagccgccctagcggtcgccgatgatagtgaaagtaagatagttatcaagccagtaaaagtggatagcaagaacagaagctacaacgtgctgccggacgaggtaaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcagccagacgcgcggcctccggcagcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggatggactggtggagtttgtgcgcgagttcgccccccggcggcgcgtgcagtggcgcgggcggaaagtgaagccggtgctgcggccaggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgcccgcggcttccaccgccacccgccgaagacgcaagtacggcgcggccagcctgctgatgcccaactacgcgctgcatccttccatcatccccacgccgggctaccgcggcacgcgcttctaccgcggctacaccagccgccgccgcaagaccaccacccgccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgccnnnggaccgcgaggacaacacctactcgtacaaagtgcgctacacgctggccgtgggcgacanccgcgtgctggacatggccagcacntactttgacatccgcggcgtgctggatcggggccccagtttcaaaccntactccggcaccgcctacannngcctggctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgataccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtgggcaggctaacataaaagattctgaaactgccagcactactcctaactatgatatagatttggcattctttgacagcaaaaatattgccgctaactatgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctgaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtgggctttacagatacttaccagggtgttaaagttaagacagatacagccgctgctggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccggccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgttgtccgagtgcaccagccccaccgcggcgtcatcgaggccgtctacctgcgcacgcccttctcggccggcaacgccaccacctaagccccgctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgtctactttcgctcccaccgcgcgcgcatcgagaaggccaccgccttcgaccgcatgaatcaagacatgtaaactgtgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccatatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcagccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgagacgatcggcaggctcagggggttcttcaccgttgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcgtgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgcagccgacgaaaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctccgaggacgagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgttacttgctgcgcctggccaactacctggcctaccactcggacgtgatcgaggacgtcagcggcgagggtctgctcgaatgccactgccgctgcaacctctgcacgccgcaccgctccctggcctgcaacccccagctgctgagcgaaacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaggaagacgaggtggaggaggaggcagaggaagaagcagccgccgccagaccgtcgtcctcggcggaggaggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagctagaaaatccacagcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacatacccgtctgaccctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgccgctccccgctactaacaaccaaactacccaccaacgccgccgtcgcgacctttctgaatctaatactaccacccacaccggaggtgagctccgaggtcgaccaacctctgggatttactacggcccctgggaggtggtagggttaatagcgctaggcctagttgcgggtgggcttttggctctctgctacctatacctcccttgctgttcgtacttagtggtgctgtgttgctggtttaagaaatggggaagatcaccctagtgagctgcggtgtgctggtggcggtggtggtgctttcgattgtgggactgggcggcgcggctgtagtgaaggaggagaaggccgatccctgcttgcatttcaatcccgacaaatgccagctgagttttcagcccgatggcaatcggtgcacggtgctgattaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcacacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattaacatgttcatcgctattcgccccagaaataatgccgaaaaagagaaacagccataacacgtttttttcacacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcattttggacatggaattttatcaagtttctgtgtctgaacccaccacgcctagaatgaccacaaccacaaaaactacacctgttaccactatacagctcactaccaatggctttcttgccatgcttcaagtggctgaaaatagcaccagcattcaacccaccccacccagtgaggaaattcccagatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggttgctcgtcagcggtattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgtttttagtgctgggtttttgaaaaatcttaccatttatgaaggtgagaatgccactctagtgggcatcagtggtcaaaatgtcagctggctaaaataccatctagatgggtggaaagacatttgcgattggaatgtcactgtgtatacatgtaatggagttaacctcaccattactaatgccacccaagatcagaatggtaggtttaagggccagagtttcactagaaataatgggtatgaatcccataacatgtttatctatgacgtcactgtcatcagaaatgagactgccaccacacagatgcccactacacacagttctaccactactaccatgcaaaccacacagacaaccactacatcaactcagcatatgaccaccactacagcagcaaagccaagtagcgcagcgcctcagccacaggctttggctttgaaagctgcgcaagctagtacaactactaggaccaatgagcagactactgattttttgtccactgtcgagagccacaccacagctacctcgagtgccttctctagcaccgccaatctctcctcgctttcctctacaccaatcagtcccgctactactcctagccccgctcatcttcccactcccctgaagcaaactgaggacagcggcatgcaatggcagatcaccctgctcattgtgatcgggttggtcatcctggccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcatcacctgcatctgctgctgtagcatagtctgcctgcttatcaccttcttccagttcattgactggatctttgtgcgcatcgcctacctgcgccaccacccccagtaccacgaccagcgagtggcgcggctgctcaggctcctctgataagcatgcgggctctgctacttctcgcgcttctgctgttagtgctcccccgtcccgtcgacccccggtcccccactcagtcccccgaggaggttcgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcagcaacctcaggcacacgcactaccaccaccacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcactccaaatgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgatttaaataaaaaaataatcatttgatttgaaataaagatacaatcatattgatgatttgagtttaacaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcatcccttttacactaaagatggaaaattatccttacaagtttctccaccattaaatatactgagaacaagcattctaaacacactagctttaggttttggatcaggtttaggactccgtggctctgccttagcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaacggagatgtttcaaaacctatgcttctcactataaccctcaatggtactgatgacagcaacagtacatattcaatgtcattttcatacacctggactaatggaagctatgttggagcaacatttggggctaactcttataccttctcctacattgcccaagaatgaacactgtatcccaccctacattgcccaacccttcccaccccactctgtctatggaaaaaactctgaaacacaaaataaaataaagttcaagtgttttattgattcaacagttttacaggattcgagcagttatttttcctccaccctcccaagacatggaatacaccaccctctccccccgcacagccttgaacatttgaaagccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcaaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgtttaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagggaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtcccggcgcttggaacgctcggtgttgaagttgtaaaacagccactctcttagaccgtgcagcaaatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcaaaagcgggagggttctctaattcctcaatcatcatgttacactcctgcaccatccctagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtacgtgcaaggggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcggccgaggaagcaataaggcactacaatgctcagtctcaagtccagcaaagcgatgccatgcggatgaagcacaaaattctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaagactgagctctaacctgtccacccgctctctgctcaatatatagcccagatctacactgacgtaaaggccaaagtctaaaaatacccgccaaataatcacacacgcccagcacacgcccagaaaccggtgacacactcagaaaaatacgcgcacttcctcaaacgcccaaactgccgtcatttccgggttcccacgctacgtcatcagaattcgactttcaaattccgtcgaccgttaaaaatgtcacccgccccgcccctaacggtcgccgctcccacagccaatcacagccccgcatccccaaattcaaacagctcatttgcatattaacgcgcaccaaaagtttgaggtatattattgatgatg

SEQ ID NO: 5 - DNA, 34874 bp, Artificial Sequence
  Synthesized
catcatcaataatatacctcaaacttttggtgcgcgttaatatgcaaatgagctgtttgaatttggggatgcggggctgtgattggctgtgggagcggcgaccgttaggggcggggcgggtgacgttttgatgacgtgtttgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacatcatttccccgaaaagtgccacctgacgtaactataacggtcctaaggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcaatcgattcaggtgtaatggagtttcacgcccttcagcacaggctcggaatcgtcctcatcgaatttacagcaggatccacagctacagcagcccttcaggcaagaacagcaggatgtcatacagcacagcatgatggtcaccatcacgatagcgatcagtccggcgatgaagcccagccagatgtaccaaggccacttgatgtactgctcgtacttgcccagctcctgcagatcgatcaggctctcgttcaggttcttagccacctcgttcagcctgtcgatctccttctggatgttcaccacggaggcgttgattccgctgatgtcgcccagatccacgtcgggggaggtgtggttcttgaagtacttatccagctcctccttaaagctgtccagctctggctgcagaggatcgtacacggtgttgttcacgattccgatgaccacgtcacagttgccagacacgaatgtgttgtctgtggtgatgatctgtggctcgtagaagttgcgctgtgtcacaaaccagtgggttccgttggacacgaacacgccctcccgaggaaagtgggccttgccatcgtggcagatggcgggagctgtggtgaagttcttctcctgagctggcacgtaggtcacgtgcagaaacaccactccgtgaggggcagactgggggaaggacatcaggtggtatcccttgccacaaaagtccactctcttagactggcccagcacgcactcggacatcttggtggcagccaggttggcgctagccctgatctcagcagccctgatcagctgctgtgtcacgtaggtctgcagagactgcagtcttcctgtgatcagcctgtcgatctgcacctcagcctctggagggtccagcctgctcaggatgtcgttcagcacgctagagatggcgccgaagttggagctcagctgcttcaccagggtgttcagggcctgagcgttctggttcaccacatcctgcagctttcccagggcggaagctgtagaggacaggctgtcctggatcttgccgatagcggagttaaactggttggcgatcagcttctggttctcgtacagcacgttctgggtcactccgatgccgttgaaccggtaagccatctgcatggcaaaggggatctgcagggcagctccagcgccgaaggtccatccggatgtgatggttccagccagcagggcgcttgtgtactgagcgatcatctcatctgtcagcaggggtggcagcacggtcagtccgttaaacttctgggcacagatcaggtccctggcagcgatgtctcccaggcaatcgccgtactgcttgatgaagccggcatcagccagggtcaccttgttgaacagcaggtcctcgataaagctcctcttagatggcttggaaggatcgggcaggatctgggaaaagttgaatccgccaaagtccttgatagggggggtcttgtagatctgcttcacctgggcgaacacctcctgtgtgttcttgtcctgctccacagcgattcctgtcagggctctgttcagctgggtacaaaagctgccgtactgcagcagcaggttgctgcactcggtagaatctccacagatgtacattgtgcagtccacagatgtcttggtcatggacactggcaggatctctgtggtcacgctaattgtgaagttggtagggatagcgatgctgttgttagagtaagccacagagttctccacgcccagggacatggtgtaggcgatgatagactgggaagccacgctcctggctctccttggagagtttgtctgggtctggtaagaagcacagattccggcgccgatagggatgtcgcactcgtaggagttgttcacgtgctcagctccgatcagacagccggctcttgtctggaacacgttggagccggtgctgtacaccctccatgtaggggtcagctgatcggcgtggatagccacgggcacctcggtacagttcacgccctggtacagcacggccacctggttgcttgtgttggttcctggtgtgatcacgctcactccgccgaaagagcatggtgtgatgtccaggatctccagggtctgagggtcgcgcacagcgtctgtggtatcggcgatgtcccggccgaactgctgaaaaggcaggaacttcttgttggactcggtcagcactcctgtgccggtcagtccgttaaagttgaagttaacgcacttgttcttcaccaggttggtggacttctttggtccgcacactgtagcaggagcgtgcagcagctcaaagctcagcaccaccacgcggtagggctggtatcccacgccgtatgttggctggaatccgtaggactgcagagggaagtaacagttgaagcccttcactccgttgcatggggtgcttccagcctggtagatctctgtagagatgtcccgctcgaatggcttcaggttgctctttctaaacagcctgtacaggtagttgtagtttccgcccactttggaatccaggttgttgctgttccaggcgatcacgcagccggtgaaatcgtcaggcagcttgtagttgtagtcagcgatgtttcctgtctggccgggagcgatctggcgcacctcgtctcccctgatcacgaaagaatcggcgtacacgttggtaaagcacaggtcgttcagctttgtaggggacacgccgtagcacttaaaggtgctgaaagaagcggagttgtacagcacgctgtagtcggccacgcagttagagatgcgcttccggttccaagcgtacacgctggcgaagcgggtagcgttgaacacctctccaaaagggcacaggtttgtgatgttgggaaagcgcacgatagactcggtaggctgcacccggaagttactagtctggtagatgcccttctccacggtaaaggacttcagtgtacactttgtctcgctcagggggtccagggcgcaatccacagcgtctgtgatggttccgttctcgttgtacttcagcaggaaggtccggggctgcaggtagcccacgtagtaagcagcagctccagcggtccatccgctagaggagtcgcctggtgtcaggtagcttctgtgcagtgtctgaaacctggtgatgttgattccgatgggcagatccaccagtggctccagggcgctgaagccctgtggcaggccgcgcaccaggttgattggggtgtgcttagagtagatcttaaagtagccgtcgatgttcttaaacacgaactcccgcaggttcttgaagtttccctgcttgccctccaggtccatcaggaagggctgggacacgtactcaaatgtgcagttgttggcgctagagtacactctaaactcggactccatccagctcttgttgttcttgtggtagtacacgcccaggaatggatcgttacaaaactggaactcgcacaccttgatgaccacgttggtggcgttgttcacgatcagcagagactgtgtcttggagtccagtgtggttccaaagatccagcctctgatgatgttgctcttctcggtagaggcgaagtacacgccatcgttaaagggcagcactgggttggcgaacctctttgttccgttggtgccagacacgtggatagcgtggaaccaggtcacgttgctaaagaaaggcagaaacagatcctgtgtagagtgcagcacggagcttctaaacaccttgtcggggtaatacactcctcttgtgaaggagttggtgtaagcgggagggagctgggtccgagtagtcaggttcacgcactgggatgagacgagagggaggagcacgagaaagacaaacatggtggcggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagaggcctgaccatctggtgctggcctgcaccagggccgagtttgggtctagcttaagtttgatccgatctttttccctctgccaaaaattatggggacatcatgaagccccttgagcatctgacttctggctaataaaggaaatttattttcattgcaatagtgtgttggaattttttgtgtctctcactcggaagcgatctgaattcatctatgtcgggtgcggagaaagaggtaatgaaatggcattatgggtattatgggtctgcattaatgaatcggccagattatgctggccaccgtgcatgtggcctcgcacccccgcaagacatggcccgagttcgagcacaacgtcatgacccgatgcaacgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgggagtctgcatgatgggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcaaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggctctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgcccgcccaggttttccatgcactcatccatgatgatggcgatgggcccgtgggcggcggcctgggcaaagacgtttcgggggtcggacacatcatagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtgggaccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagttcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcatgccccagggcatggggtgcgtgagcgcagaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgcgccgaggttggagcgctgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttgatgttgtggcccacgatgtagagttccacgaaccgtgggcggcccttgacgtggggcagcttcttgagctcctcgtaggtgagctcgtcagggtcgctgagcccgtgctgctcgagggcccagtcggcgagatggtggttggcgcggaggaaggaagtccagagatccacggccagggcggtttgcagacggtcccggtactgacggaactgctggccgacggccattttttcgggggtgacgcagtagaaggtgcgggggtccccgtgccagcgatcccatttgagctggagggcgagatcgagggcgagctcgacgaggcggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggagtgaccaccgtcccccgtttcttcttgggcgctgcttccatgccggtcagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggccccgggaacctcttcttccatctcctcttcttcctcttccactaacatctcttctacttcctcctcaggcggtggcgggggagggggcctgcgtcgccggcggcgcacgggcagacggtcgatgaagcgctcgatggtctcgccgcgccggcgtcgcatggtctcggtgacggcccgcccgtcctcgcggggccgcagcgtgaagacgccgccgcgcatttccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctggcgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccagatccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggccgggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgtttttgcaactttttttcggaggccggaaatgaagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagacccctagccagccgacttctccagttacggagcgagcccctcttttgttttttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctaaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgagcggtgagcgccagcaggcggcgcgagctgagcgaccaggagctgatgcacagcctgcagcgggccctgaccggggccgggaccgagggggagagctactttgacatgggcgcggacctgcactggcaacccagccgccgggccttggaggcggcggcaggaccctacgtagaagaggtggacgatgaggtggacgagggcgagtacctggaagactgatggcgcgaccgtatttttgctagatgcaacaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagaccaacctggacaggatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagtatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacgcgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctaaacccccactggctaccgccgccggggttctacacgggcgagtacgacatgcccgaccccaatgacgggttcctgtgggacgatgtggacagcagcgtgttttccccccgaccgggtgctaacgagcgccccttgtggaagaaggaaggcagcgaccgacgcccgtcctcggcgctgtccggccgcgagggtgctgccgcggcggtgcccgaggccgccagtccttttcctagcttgcccttctcgctgaacagtattcgcagcagcgagctgggcaggatcacgcgcccgcgcttgctcggcgaggaggagtacttgaatgactcgctgttgagacccgagcgggagaagaacttccccaataacgggatagagagcctggtggacaagatgagccgctggaagacgtacgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtggtggtaacccgttcgctcacctgcgcccccgcatcgggcgcatgatgtaagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgaattcatgtacagcaacaagttcaaggcgcgggtcatggtctcccgcaagacccccaatggggtcaaagtagatgaaaattatgatggtagtcaggatgagctgaaatacgagtgggtggagtttgagctgcccgaaggcaacttctcggtgaccatgaccatcgacctgatgaacaacgccatcatcgacaattacttggcggtggggcggcagaacggggtcctggaaagcgacatcggcgtgaagttcgacactaggaacttcaggctgggctgggaccccgtgaccgagctggtcatgcccggggtgtacaccaacgaggccttccatcccgatgttgtcttgctgcccggctgcggggtggactttaccgagagccgcctcagcaacctgctgggcattcgcaagaggcagcccttccaggagggattccagatcatgtacgaggatctggaggggggcaacatccccgcgctcctggatgtcgaggcctatgaggaaagcaaggaaaaagctgaagccgaggcgactgcagccgtggctaccgccgcgaccaccaatgcagatgcaactaccaccagaggcgatacattcgccactgtggcggaggaagcagccgccctagcggtcgccgatgatagtgaaagtaagatagttatcaagccagtaaaagtggatagcaagaacagaagctacaacgtgctgccggacgaggtaaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcagccagacgcgcggcctccggcagcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggatggactggtggagtttgtgcgcgagttcgccccccggcggcgcgtgcagtggcgcgggcggaaagtgaagccggtgctgcggccaggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgcccgcggcttccaccgccacccgccgaagacgcaagtacggcgcggccagcctgctgatgcccaactacgcgctgcatccttccatcatccccacgccgggctaccgcggcacgcgcttctaccgcggctacaccagccgccgccgcaagaccaccacccgccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccccagtttcaaaccctactccggcaccgcctacaacagcctggctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgataccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtgggcaggctaacataaaagattctgaaactgccagcactactcctaactatgatatagatttggcattctttgacagcaaaaatattgccgctaactatgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctgaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtgggctttacagatacttaccagggtgttaaagttaagacagatacagccgctgctggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccggccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgttgtccgagtgcaccagccccaccgcggcgtcatcgaggccgtctacctgcgcacgcccttctcggccggcaacgccaccacctaagccccgctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgtctactttcgctcccaccgcgcgcgcatcgagaaggccaccgccttcgaccgcatgaatcaagacatgtaaactgtgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccatatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcagccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgagacgatcggcaggctcagggggttcttcaccgttgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcgtgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgcagccgacgaaaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctccgaggacgagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgttacttgctgcgcctggccaactacctggcctaccactcggacgtgatcgaggacgtcagcggcgagggtctgctcgaatgccactgccgctgcaacctctgcacgccgcaccgctccctggcctgcaacccccagctgctgagcgaaacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaggaagacgaggtggaggaggaggcagaggaagaagcagccgccgccagaccgtcgtcctcggcggaggaggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagctagaaaatccacagcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacatacccgtctgaccctgtactgcatctgcaaccaccccggcctgcatgaaagtctttgttgtctgctgtgtactgagtataataaaagctgagatcagcgactactgcgatcgctcacccccttatccagtgaaataaagatcatattgatgatgatttaaataaaaaaataatcatttgatttgaaataaagatacaatcatattgatgatttgagtttaacaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcatcccttttacactaaagatggaaaattatccttacaagtttctccaccattaaatatactgagaacaagcattctaaacacactagctttaggttttggatcaggtttaggactccgtggctctgccttagcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaacggagatgtttcaaaacctatgcttctcactataaccctcaatggtactgatgacagcaacagtacatattcaatgtcattttcatacacctggactaatggaagctatgttggagcaacatttggggctaactcttataccttctcctacattgcccaagaatgaacactgtatcccaccctacattgcccaacccttcccaccccactctgtctatggaaaaaactctgaaacacaaaataaaataaagttcaagtgttttattgattcaacagttttacaggattcgagcagttatttttcctccaccctcccaagacatggaatacaccaccctctccccccgcacagccttgaacatttgaaagccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcaaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgtttaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagggaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtcccggcgcttggaacgctcggtgttgaagttgtaaaacagccactctcttagaccgtgcagcaaatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcaaaagcgggagggttctctaattcctcaatcatcatgttacactcctgcaccatccctagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtacgtgcaaggggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcggccgaggaagcaataaggcactacaatgctcagtctcaagtccagcaaagcgatgccatgcggatgaagcacaaaattctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaagactgagctctaacctgtccacccgctctctgctcaatatatagcccagatctacactgacgtaaaggccaaagtctaaaaatacccgccaaataatcacacacgcccagcacacgcccagaaaccggtgacacactcagaaaaatacgcgcacttcctcaaacgcccaaactgccgtcatttccgggttcccacgctacgtcatcagaattcgactttcaaattccgtcgaccgttaaaaatgtcacccgccccgcccctaacggtcgccgctcccacagccaatcacagccccgcatccccaaattcaaacagctcatttgcatattaacgcgcaccaaaagtttgaggtatattattgatgatg

SEQ ID NO: 6 - DNA, 34836 bp, Artificial Sequence
  Synthesized
catcatcaataatatacctcaaacttttggtgcgcgttaatatgcaaatgagctgtttgaatttggggatgcggggctgtgattggctgtgggagcggcgaccgttaggggcggggcgggtgacgttttgatgacgtgtttgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacatcatttccccgaaaagtgccacctgacgtaactataacggtcctaaggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcaatcgattcaggtgtaatggagtttcacgccctcaggatccacagctacagcagcccttcaggcaagaacagcaggatgtcatacagcacagcatgatggtcaccatcacgatagcgatcagtccggcgatgaagcccagccagatgtaccaaggccacttgatgtactgctcgtacttgcccagctcctgcagatcgatcaggctctcgttcaggttcttagccacctcgttcagcctgtcgatctccttctggatgttcaccacggaggcgttgattccgctgatgtcgcccagatccacgtcgggggaggtgtggttcttgaaatacttatccagctcctccttaaagctgtccagctctggctgcagaggatcgtacacggtgttgttcacgattccgatgaccacgtcacagttgccagacacgaatgtgttgtctgtggtgatgatctgtggctcgtagaagttgcgctgtgtcacaaaccagtgggttccgttggacacgaacacgccctcccgaggaaagtgggccttgccatcgtggcagatggcgggagctgtggtaaagttcttctcctgagctggcacgtaggtcacgtgcagaaacacgactccgtgaggggcagactgggggaaggacatcaggtggtatcccttgccacaaaagtccactctcttagactggcccagcacgcactcggacatcttggtggcagccaggttggcgctagccctgatctcagcagccctgatcagctgctgtgtcacgtaggtctgcagagactgcagtcttcctgtgatcagcctgtcgatctgcacctcagcctctggagggtccagcctgctcaggatgtcgttcagcacgctagagatggcgccgaagttggagctcagctgcttcaccagggtgttcagggcctgagcgttctggttcaccacatcctgcagctttcccagggcggaagctgtagaggacaggctgtcctggatcttgccgatagcggagttaaactggttggcgatcagcttctggttctcgtacagcacgttctgggtcactccgatgccgttgaaccggtaagccatctgcatggcaaaggggatctgcagggcagctccagcgccgaaggtccatccggatgtgatggttccagccagcagggcgcttgtgtactgagcgatcatctcatctgtcagcaggggtggcagcacggtcagtccgttaaacttctgggcacagatcaggtccctggcagcgatgtctcccaggcaatcgccgtactgcttgatgaagccggcatcagccagggtcaccttgttgaacagcaggtcctcgataaagctcctcttagatggcttggaaggatcgggcaggatctgggaaaagttgaatccgccaaagtccttgatagggggggtcttgtagatctgcttcacctgggcgaacacctcctgtgtgttcttgtcctgctccacagcgattcctgtcagggctctgttcagctgggtacaaaagctgccgtactgcagcagcaggttgctgcactcggtagaatctccacagatgtacattgtgcagtccacagatgtcttggtcatggacactggcaggatctctgtggtcacgctaattgtgaagttggtagggatagcgatgctgttgttagagtaagccacagagttctccacgcccagggacatggtgtaggcgatgatagactgggaagccacgctcctggctctccttggagagtttgtctgggtctggtaagaagcacagattccggcgccgatagggatgtcgcactcgtaggagttgttcacgtgctcagctccgatcagacagccggctcttgtctggaacacgttggagccggtgctgtacaccctccatgtaggggtcagctgatcggcgtggatagccacgggcacctcggtacagttcacgccctggtacagcacggccacctggttgcttgtgttggttcctggtgtgatcacgctcactccgccgaaagagcatggtgtgatgtccaggatctccagggtctgagggtcgcgcacagcgtctgtggtatcggcgatgtcccggccgaactgctgaaaaggcaggaacttcttgttggactcggtcagcactcctgtgccggtcagtccgttaaagttgaagttaacgcacttgttcttcaccaggttggtggacttctttggtccgcacactgtagcaggagcgtgcagcagctcaaagctcagcaccaccacgcggtagggctggtatcccacgccgtatgttggctggaatccgtaggactgcagagggaagtaacagttgaagcccttcactccgttgcatggggtgcttccagcctggtagatctctgtagagatgtcccgctcgaatggcttcaggttgctctttctaaacagcctgtacaggtagttgtagtttccgcccactttggaatccaggttgttgctgttccaggcgatcacgcagccggtgaaatcgtcaggcagcttgtagttgtagtcagcgatgtttcctgtctggccgggagcgatctggcgcacctcgtctcccctgatcacgaaagaatcggcgtacacgttggtaaagcacaggtcgttcagctttgtaggggacacgccgtagcacttaaaggtgctgaaagaagcggagttgtacagcacgctgtagtcggccacgcagttagagatgcgcttccggttccaagcgtacacgctggcgaagcgggtagcgttgaacacctctccaaaagggcacaggtttgtgatgttgggaaagcgcacgatagactcggtaggctgcacccggaagttactagtctggtagatgcccttctccacggtaaaggacttcagtgtacactttgtctcgctcagggggtccagggcgcaatccacagcgtctgtgatggttccgttctcgttgtacttcagcaggaaggtccggggctgcaggtagcccacgtagtaagcagcagctccagcggtccatccgctagaggagtcgcctggtgtcaggtagcttctgtgcagtgtctgaaacctggtgatgttgattccgatgggcagatccaccagtggctccagggcgctgaagccctgtggcaggccgcgcaccaggttgattggggtgtgcttagagtagatcttaaagtagccgtcgatgttcttaaacacgaactcccgcaggttcttgaagtttccctgcttgccctccaggtccatcaggaagggctgggacacgtactcaaatgtgcagttgttggcgctagagtacactctaaactcggactccatccagctcttgttgttcttgtggtagtacacgcccaggaatggatcgttacaaaactggaactcgcacaccttgatgaccacgttggtggcgttgttcacgatcagcagagactgtgtcttggagtccagtgtggttccaaagatccagcctctgatgatgttgctcttctcggtagaggcgaagtacacgccatcgttaaagggcagcactgggttggcgaacctctttgttccgttggtgccagacacgtggatagcgtggaaccaggtcacgttgctaaagaaaggcagaaacagatcctgtgtagagtgcagcacggagcttctaaacaccttgtcggggtaatacactcctcttgtgaaggagttggtgtaagcgggagggagctgggtccgagtagtcaggttcacgcactgggatgagacgagagggaggagcacgagaaagacaaacatggtggcggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagaggcctgaccatctggtgctggcctgcaccagggccgagtttgggtctagcttaagtttgatccgatctttttccctctgccaaaaattatggggacatcatgaagccccttgagcatctgacttctggctaataaaggaaatttattttcattgcaatagtgtgttggaattttttgtgtctctcactcggaagcgatctgaattcatctatgtcgggtgcggagaaagaggtaatgaaatggcattatgggtattatgggtctgcattaatgaatcggccagattatgctggccaccgtgcatgtggcctcgcacccccgcaagacatggcccgagttcgagcacaacgtcatgacccgatgcaacgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgggagtctgcatgatgggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcaaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggctctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgcccgcccaggttttccatgcactcatccatgatgatggcgatgggcccgtgggcggcggcctgggcaaagacgtttcgggggtcggacacatcatagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtgggaccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagttcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcatgccccagggcatggggtgcgtgagcgcagaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgcgccgaggttggagcgctgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttgatgttgtggcccacgatgtagagttccacgaaccgtgggcggcccttgacgtggggcagcttcttgagctcctcgtaggtgagctcgtcagggtcgctgagcccgtgctgctcgagggcccagtcggcgagatggtggttggcgcggaggaaggaagtccagagatccacggccagggcggtttgcagacggtcccggtactgacggaactgctggccgacggccattttttcgggggtgacgcagtagaaggtgcgggggtccccgtgccagcgatcccatttgagctggagggcgagatcgagggcgagctcgacgaggcggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggagtgaccaccgtcccccgtttcttcttgggcgctgcttccatgccggtcagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggccccgggaacctcttcttccatctcctcttcttcctcttccactaacatctcttctacttcctcctcaggcggtggcgggggagggggcctgcgtcgccggcggcgcacgggcagacggtcgatgaagcgctcgatggtctcgccgcgccggcgtcgcatggtctcggtgacggcccgcccgtcctcgcggggccgcagcgtgaagacgccgccgcgcatttccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctggcgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccagatccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggccgggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgtttttgcaactttttttcggaggccggaaatgaagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagacccctagccagccgacttctccagttacggagcgagcccctcttttgttttttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctaaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgagcggtgagcgccagcaggcggcgcgagctgagcgaccaggagctgatgcacagcctgcagcgggccctgaccggggccgggaccgagggggagagctactttgacatgggcgcggacctgcactggcaacccagccgccgggccttggaggcggcggcaggaccctacgtagaagaggtggacgatgaggtggacgagggcgagtacctggaagactgatggcgcgaccgtatttttgctagatgcaacaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagaccaacctggacaggatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagtatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacgcgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctaaacccccactggctaccgccgccggggttctacacgggcgagtacgacatgcccgaccccaatgacgggttcctgtgggacgatgtggacagcagcgtgttttccccccgaccgggtgctaacgagcgccccttgtggaagaaggaaggcagcgaccgacgcccgtcctcggcgctgtccggccgcgagggtgctgccgcggcggtgcccgaggccgccagtccttttcctagcttgcccttctcgctgaacagtattcgcagcagcgagctgggcaggatcacgcgcccgcgcttgctcggcgaggaggagtacttgaatgactcgctgttgagacccgagcgggagaagaacttccccaataacgggatagagagcctggtggacaagatgagccgctggaagacgtacgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtggtggtaacccgttcgctcacctgcgcccccgcatcgggcgcatgatgtaagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgaattcatgtacagcaacaagttcaaggcgcgggtcatggtctcccgcaagacccccaatggggtcaaagtagatgaaaattatgatggtagtcaggatgagctgaaatacgagtgggtggagtttgagctgcccgaaggcaacttctcggtgaccatgaccatcgacctgatgaacaacgccatcatcgacaattacttggcggtggggcggcagaacggggtcctggaaagcgacatcggcgtgaagttcgacactaggaacttcaggctgggctgggaccccgtgaccgagctggtcatgcccggggtgtacaccaacgaggccttccatcccgatgttgtcttgctgcccggctgcggggtggactttaccgagagccgcctcagcaacctgctgggcattcgcaagaggcagcccttccaggagggattccagatcatgtacgaggatctggaggggggcaacatccccgcgctcctggatgtcgaggcctatgaggaaagcaaggaaaaagctgaagccgaggcgactgcagccgtggctaccgccgcgaccaccaatgcagatgcaactaccaccagaggcgatacattcgccactgtggcggaggaagcagccgccctagcggtcgccgatgatagtgaaagtaagatagttatcaagccagtaaaagtggatagcaagaacagaagctacaacgtgctgccggacgaggtaaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcagccagacgcgcggcctccggcagcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggatggactggtggagtttgtgcgcgagttcgccccccggcggcgcgtgcagtggcgcgggcggaaagtgaagccggtgctgcggccaggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgcccgcggcttccaccgccacccgccgaagacgcaagtacggcgcggccagcctgctgatgcccaactacgcgctgcatccttccatcatccccacgccgggctaccgcggcacgcgcttctaccgcggctacaccagccgccgccgcaagaccaccacccgccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccccagtttcaaaccctactccggcaccgcctacaacagcctggctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgataccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtgggcaggctaacataaaagattctgaaactgccagcactactcctaactatgatatagatttggcattctttgacagcaaaaatattgccgctaactatgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctgaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtgggctttacagatacttaccagggtgttaaagttaagacagatacagccgctgctggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccggccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgttgtccgagtgcaccagccccaccgcggcgtcatcgaggccgtctacctgcgcacgcccttctcggccggcaacgccaccacctaagccccgctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgtctactttcgctcccaccgcgcgcgcatcgagaaggccaccgccttcgaccgcatgaatcaagacatgtaaactgtgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccatatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcagccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgagacgatcggcaggctcagggggttcttcaccgttgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcgtgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgcagccgacgaaaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctccgaggacgagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgttacttgctgcgcctggccaactacctggcctaccactcggacgtgatcgaggacgtcagcggcgagggtctgctcgaatgccactgccgctgcaacctctgcacgccgcaccgctccctggcctgcaacccccagctgctgagcgaaacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaggaagacgaggtggaggaggaggcagaggaagaagcagccgccgccagaccgtcgtcctcggcggaggaggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagctagaaaatccacagcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacatacccgtctgaccctgtactgcatctgcaaccaccccggcctgcatgaaagtctttgttgtctgctgtgtactgagtataataaaagctgagatcagcgactactgcgatcgctcacccccttatccagtgaaataaagatcatattgatgatgatttaaataaaaaaataatcatttgatttgaaataaagatacaatcatattgatgatttgagtttaacaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcatcccttttacactaaagatggaaaattatccttacaagtttctccaccattaaatatactgagaacaagcattctaaacacactagctttaggttttggatcaggtttaggactccgtggctctgccttagcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaacggagatgtttcaaaacctatgcttctcactataaccctcaatggtactgatgacagcaacagtacatattcaatgtcattttcatacacctggactaatggaagctatgttggagcaacatttggggctaactcttataccttctcctacattgcccaagaatgaacactgtatcccaccctacattgcccaacccttcccaccccactctgtctatggaaaaaactctgaaacacaaaataaaataaagttcaagtgttttattgattcaacagttttacaggattcgagcagttatttttcctccaccctcccaagacatggaatacaccaccctctccccccgcacagccttgaacatttgaaagccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcaaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgtttaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagggaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtcccggcgcttggaacgctcggtgttgaagttgtaaaacagccactctcttagaccgtgcagcaaatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcaaaagcgggagggttctctaattcctcaatcatcatgttacactcctgcaccatccctagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtacgtgcaaggggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcggccgaggaagcaataaggcactacaatgctcagtctcaagtccagcaaagcgatgccatgcggatgaagcacaaaattctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaagactgagctctaacctgtccacccgctctctgctcaatatatagcccagatctacactgacgtaaaggccaaagtctaaaaatacccgccaaataatcacacacgcccagcacacgcccagaaaccggtgacacactcagaaaaatacgcgcacttcctcaaacgcccaaactgccgtcatttccgggttcccacgctacgtcatcagaattcgactttcaaattccgtcgaccgttaaaaatgtcacccgccccgcccctaacggtcgccgctcccacagccaatcacagccccgcatccccaaattcaaacagctcatttgcatattaacgcgcaccaaaagtttgaggtatattattgatgatg

SEQ ID NO: 7 - DNA, 40 bp, Artificial Sequence
  Synthesized
tctctatcactgatagggagatctctatcactgataggga

SEQ ID NO: 8 - DNA, 20 bp, Artificial Sequence
  Synthesized
atgctgcaatcgtgctacaa

SEQ ID NO: 9 - DNA, 17 bp, Artificial Sequence
  Synthesized
gactgccgcctctgctc